(19) United States
(12) Patent Application Publication
(10) Pub. No.: US 2026/0070886 A1
(43) Pub. Date: Mar. 12, 2026

(54) QUINOLINE PIPERAZINE DERIVATIVES, PHARMACEUTICAL COMPOSITION AND USES THEREOF

(71) Applicant: Immunocure Inc, Livingston, NJ (US)

(72) Inventors: Ravikumar Muttineni, Hyderabad (IN); Kranthi Raj KODAMALA, Medchalmalkajgiri (IN)

(73) Assignee: Immunocure Inc, Livingston, NJ (US)

(21) Appl. No.: 19/107,640

(22) PCT Filed: Aug. 27, 2023
(86) PCT No.: PCT/US2023/031208

§ 371 Date: Feb. 28, 2025

Related U.S. Application Data
(60) Provisional application No. 63/402,588, filed on Aug. 31, 2022.

(51) Int. Cl. C07D 401/12; A61K 31/506; A61K 31/517; A61K 31/5377; A61K 31/541; A61K 31/55; A61P 35/00; C07D 401/14; C07D 403/12; C07D 409/14; C07D 471/04

(52) U.S. Cl. CPC C07D 401/12; A61K 31/506; A61K 31/517; A61K 31/5377; A61K 31/541; A61K 31/55; A61P 35/00; C07D 401/14; C07D 403/12; C07D 409/14; C07D 471/04

(57) ABSTRACT

The present invention provides a novel compound of Formula A or stereoisomer, tautomers, solvates, hydrates, pharmaceutically acceptable salts thereof. Said Formula A in pharmaceutical compositions has application in treating cancer including lung cancer.

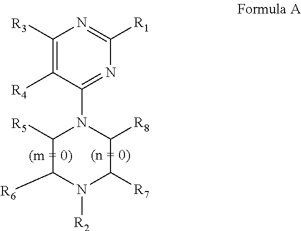

### RELATED APPLICATIONS

This application claims priority from the PCT application No. PCT/US2023/031208 filed on Aug. 27, 2023, which claims priority to the US provisional patent application numbered 63/402,588 titled “QUINOLINE PIPERAZINE DERIVATIVES, PHARMACEUTICAL COMPOSITION AND USES THEREOF” filed on Aug. 31, 2022. Both applications are incorporated in full herein by reference FIELD OF THE INVENTION

The present invention relates to the field of anti-cancer compounds, more specifically, novel anti-cancer compounds represented by Formula A, quinoline piperazine derivatives. The invention further relates to processes for the preparation of Formula 1, their pharmaceutical compositions thereof, and their use as anti-cancer drugs.

### BACKGROUND OF THE INVENTION

Cancer is a serious health issue that presents a huge threat to human health all over the world. Doctors and scientists across the continents are working in sync and are always looking for better ways to care for people with cancer. Global cancer statistics have predicted that the number of cancer cases will constantly increase. One way to do curb this increase is to create and study new drugs.

During the last few years, there has been continued progress in anticancer drug development. New agents ranging from small molecules to engineered antibodies and immune modulators have been approved for cancer treatment. Drugs go through a long development and approval process before any drug can be prescribed to a patient.

There are 3 main steps in developing a new drug: Preclinical research, when the drug is found and first tested; Clinical research, when the drug is tested in people, and Post-clinical research, which takes place after the drug is approved and studies continue.

The preclinical research in most of the drug discovery programs begin with screening e.g. biochemical, virtual or biophysical, for agonists, antagonists or inhibitors of a target associated to a particular disease. This is the general target-based drug discovery (TDD), in which the starting point is a defined molecular target that is hypothesized to have an important role in disease, driven by advances in molecular biology and genomics. This is followed by chemical optimization based on target potency. These compounds need further refinement into derivatives of superior potency and/or selectivity, and desirable pharmacokinetic properties. Selected drug candidates are then validated in vivo and, upon verified safety and efficacy, progressed to the second stage—clinical research, i.e., human trials. This is followed by regulatory approvals at state level and marketing. Clinical trials are used to find out if a new drug is safe, effective, and better than standard treatments. Before approval by regulatory bodies such as FDA, clinical trials for a medication must go through 3 phases. Early phases of clinical trials focus on the drug's safety, its dosing, and how the body processes the drug. Later phases study how well the drug works. Each phase involves a larger number of people than the phase before it.

This well-defined process often takes many years of research and significant resources which generally has low success rate in the development of anticancer drugs. Further, these conventional general target-based drug discovery (TDD) methods are not appropriately tailored to pathologies generated by the concurrent or sequential action of multiple etiologic factors such as cancer [1,2]. Additionally, many of these drugs still cause serious adverse side effects. Attrition is a major issue in anticancer drug development with up to 95% of drugs tested in Phase I trials not reaching a marketing authorisation making the drug development process enormously costly and inefficient.

The obstacles faced by conventional drug discovery methods has led to out-of-the-box thinking, which increased the interest in phenotypic drug discovery (PDD) approaches. PDD approaches do not rely on knowledge of the identity of a specific drug target or a hypothesis about its role in disease [3,4]. PDD approach is an effective tool to address the complexity of diseases that are poorly understood by the scientific community.

The present invention provides novel anti-cancer compounds designed using a combination of databases, including small molecule databases. Target and disease databases were used to identify off target binding, target-based toxicity, drug repurposing and compound target binding patterns. Computational tools were integrated to know the binding patterns and for molecular, quantum mechanical property identifications. ADMET (Adsorption, Distribution, Metabolism, Excretion and Toxicity) models were also generated for optimizing the molecules. Several classes of small molecules were created. The present invention provides a novel class of compounds, quinoline piperazine derivatives, which have anti-cancer properties.

### OBJECT OF THE INVENTION

The main object of the invention is to provide novel compounds, quinoline piperazine derivatives, represented by Formula A including the optically active forms, racemic mixtures, polymorphs, tautomers, solvates, hydrates, complexes, pharmaceutically acceptable salts and stereoisomers thereof.

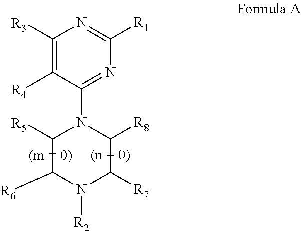

Wherein
R1, R2, R3, R4, R5, R6, R7 and R8 is selected from the group consisting of formulae provided in Table 1 to create various derivative of the parent molecule Formula A; and
m=0 and n=0, where m and n indicate the number of carbons between the indicated bonds.

Another object of the present invention is to provide novel compounds, novel compounds, quinoline piperazine derivatives, represented by Formula A, having anti-cancer properties, their pharmaceutical uses thereof.

Yet another object of the invention is to provide method for preparing the novel compounds, quinoline piperazine derivatives, represented by Formula A.

### SUMMARY OF THE INVENTION

In the main embodiment of the invention, the invention provides novel compounds, quinoline piperazine derivatives, represented by Formula A or a stereoisomer of the above compounds or pharmaceutically acceptable salt thereof:

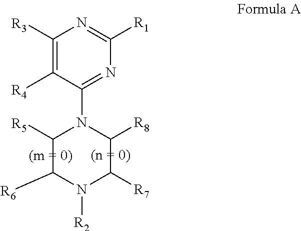

Wherein
R1, R2, R3, R4, R5, R6, R7 and R8 is selected from the group consisting of formulae provided in Table 1 to create various derivative of the parent molecule Formula A; and
m=0 and n=0, where m and n indicate the number of carbons between the indicated bonds.

TABLE 1
List of formulae representing R1-R8
R1

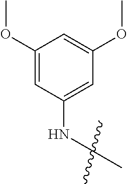 [SMILES: *Nc1cc(OC)cc(OC)c1]

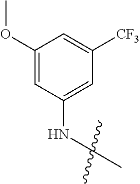 [SMILES: *Nc1cc(OC)cc(C(F)(F)F)c1]

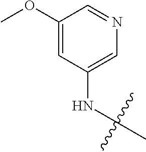 [SMILES: *Nc1cncc(OC)c1]

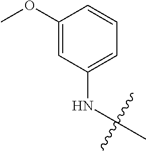 [SMILES: *Nc1cccc(OC)c1]

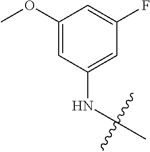 [SMILES: *Nc1cc(F)cc(OC)c1]

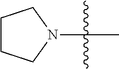 [SMILES: *N1CCCC1]

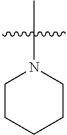 [SMILES: *N1CCCCC1]

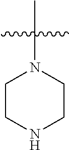 [SMILES: *N1CCNCC1]

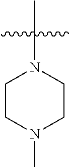 [SMILES: *N1CCN(C)CC1]

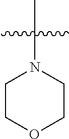 [SMILES: *N1CCOCC1]

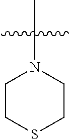 [SMILES: *N1CCSCC1]

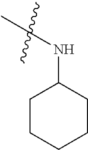 [SMILES: *NC1CCCCC1]

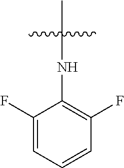 [SMILES: *Nc1c(F)cccc1F]

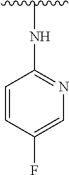 [SMILES: *Nc1ccc(F)cn1]

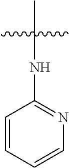 [SMILES: *Nc1ccccn1]

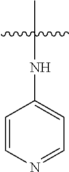 [SMILES: *Nc1ccncc1]

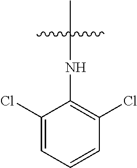 [SMILES: *Nc1c(Cl)cccc1Cl]

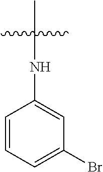 [SMILES: *Nc1cccc(Br)c1]

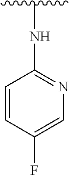 [SMILES: *Nc1ccc(F)cn1]

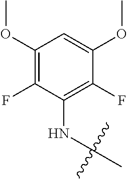 [SMILES: *Nc1c(F)c(OC)cc(OC)c1F]

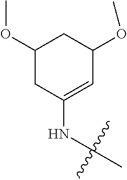 [SMILES: *NC1=CC(OC)CC(OC)C1]

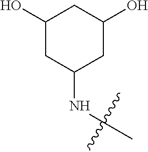 [SMILES: *NC1CC(O)CC(O)C1]

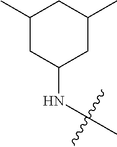

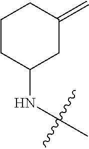 [SMILES: *NC1CCCC(=C)C1]

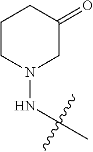 [SMILES: *NN1CCCC(=O)C1]

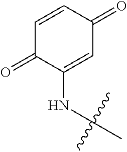 [SMILES: *NC1=CC(=O)C=CC1=O]

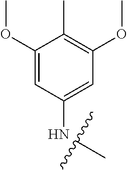

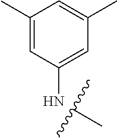

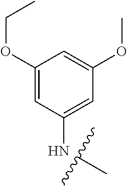

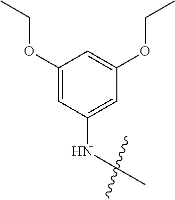 [SMILES: *Nc1cc(OCC)cc(OCC)c1]

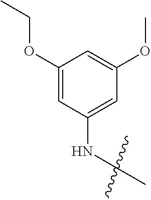 [SMILES: *Nc1cc(OC)cc(OCC)c1]

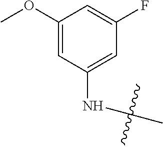 [SMILES: *Nc1cc(F)cc(OC)c1]

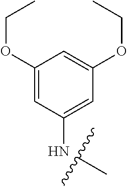

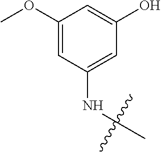 [SMILES: *Nc1cc(O)cc(OC)c1]

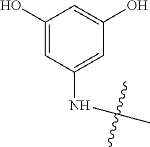 [SMILES: *Nc1cc(O)cc(O)c1]

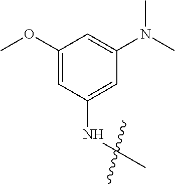

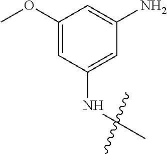 [SMILES: *Nc1cc(N)cc(OC)c1]

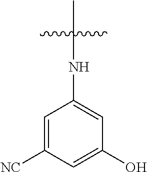 [SMILES: *Nc1cc(O)cc(C#N)c1]

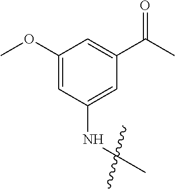 [SMILES: *Nc1cc(OC)cc(C(C)=O)c1]

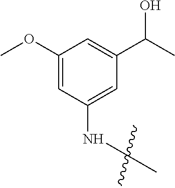 [SMILES: *Nc1cc(OC)cc(C(C)O)c1]

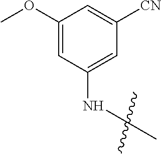 [SMILES: *Nc1cc(C#N)cc(OC)c1]

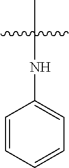 [SMILES: *Nc1ccccc1]

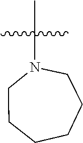 [SMILES: *N1CCCCCC1]

R2

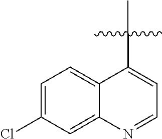 [SMILES: *c1ccnc2cc(Cl)ccc12]

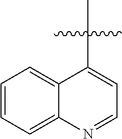 [SMILES: *c1ccnc2ccccc12]

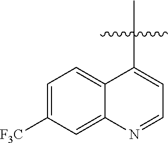 [SMILES: *c1ccnc2cc(C(F)(F)F)ccc12]

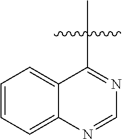 [SMILES: *c1ncnc2ccccc12]

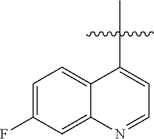 [SMILES: *c1ccnc2cc(F)ccc12]

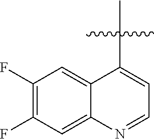 [SMILES: *c1ccnc2cc(F)c(F)cc12]

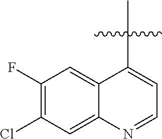 [SMILES: *c1ccnc2cc(Cl)c(F)cc12]

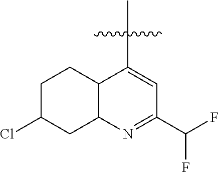 [SMILES: *C1=CC(C(F)F)=NC2CC(Cl)CCC12]

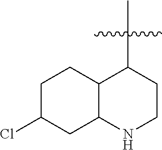 [SMILES: *C1CCNC2CC(Cl)CCC12]

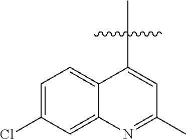 [SMILES: *c1cc(C)nc2cc(Cl)ccc12]

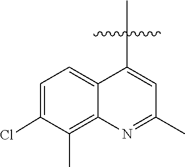 [SMILES: *c1cc(C)nc2c(C)c(Cl)ccc12]

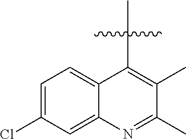 [SMILES: *c1c(C)c(C)nc2cc(Cl)ccc12]

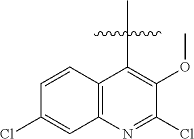 [SMILES: *c1c(OC)c(Cl)nc2cc(Cl)ccc12]

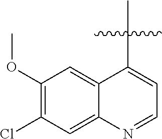 [SMILES: *c1ccnc2cc(Cl)c(OC)cc12]

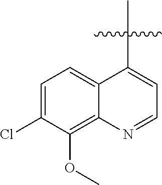 [SMILES: *c1ccnc2c(OC)c(Cl)ccc12]

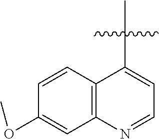 [SMILES: *c1ccnc2cc(OC)ccc12]

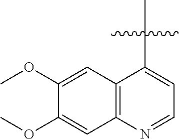 [SMILES: *c1ccnc2cc(OC)c(OC)cc12]

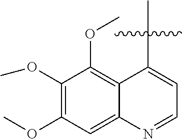 [SMILES: *c1ccnc2cc(OC)c(OC)c(OC)c12]

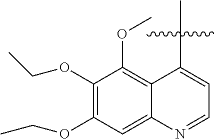 [SMILES: *c1ccnc2cc(OCC)c(OCC)c(OC)c12]

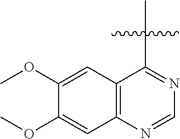 [SMILES: *c1ncnc2cc(OC)c(OC)cc12]

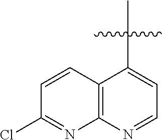 [SMILES: *c1ccnc2nc(Cl)ccc12]

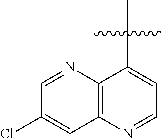 [SMILES: *c1ccnc2cc(Cl)cnc12]

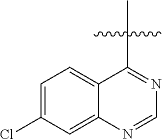 [SMILES: *c1ncnc2cc(Cl)ccc12]

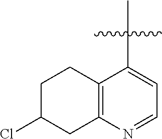 [SMILES: *c1ccnc2c1CCC(Cl)C2]

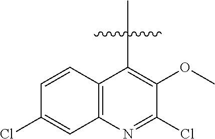 [SMILES: *c1c(OC)c(Cl)nc2cc(Cl)ccc12]

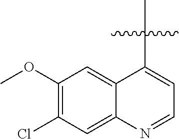 [SMILES: *c1ccnc2cc(Cl)c(OC)cc12]

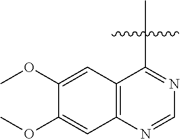 [SMILES: *c1ncnc2cc(OC)c(OC)cc12]

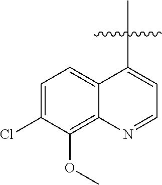 [SMILES: *c1ccnc2c(OC)c(Cl)ccc12]

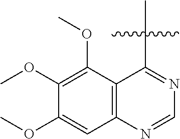 [SMILES: *c1ncnc2cc(OC)c(OC)c(OC)c12]

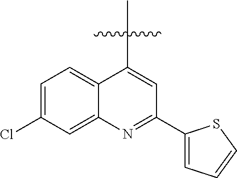 [SMILES: *c1cc(-c2cccs2)nc2cc(Cl)ccc12]

R3
CF₃
CH₃
H
F
R4
CH₃
H
F
R5
CH₃
H
R6
H
R7
CH₃
H

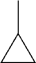

R8
CH₃
H

In another embodiment of the invention, the invention provides quinoline piperazine derivatives, represented by Formula A or a stereoisomer of the above compounds or pharmaceutically acceptable salt thereof, having anti-cancer properties.

In yet another embodiment of the invention, the invention provides method for preparing the quinoline piperazine derivatives, represented by Formula A.

### BRIEF DESCRIPTION OF DRAWINGS

FIG. 1A provides cell toxicity curve for Compound-A1 against A549 cell line.

FIG. 1B provides cell toxicity curve for Compound-A7 against A549 cell line.

FIG. 1C provides cell toxicity curve for Compound-A42 against A549 cell line.

FIG. 1D provides cell toxicity curve for Compound-A43 against A549 cell line.

FIG. 1E provides cell toxicity curve for Compound-A48 against A549 cell line.

FIG. 1F provides cell toxicity curve for Compound-A49 against A549 cell line.

FIG. 2A provides cell toxicity curve for Compound-A1 against Ncl-H 460 cell line.

FIG. 2B provides cell toxicity curve for Compound-A7 against Ncl-H 460 cell line.

FIG. 2C provides cell toxicity curve for Compound-A42 against Ncl-H 460 cell line.

FIG. 2D provides cell toxicity curve for Compound-A43 against Ncl-H 460 cell line.

FIG. 2E provides cell toxicity curve for Compound-A48 against Ncl-H 460 cell line.

FIG. 2F provides cell toxicity curve for Compound-A49 against Ncl-H 460 cell line.

FIG. 3A provides cell toxicity curve for Compound-A1 against Ncl-H 522 cell line.

FIG. 3B provides cell toxicity curve for Compound-A7 against Ncl-H 522 cell line.

FIG. 3C provides cell toxicity curve for Compound-A42 against Ncl-H 522 cell line.

FIG. 3D provides cell toxicity curve for Compound-A43 against Ncl-H 522 cell line.

FIG. 3E provides cell toxicity curve for Compound-A48 against Ncl-H 522 cell line.

FIG. 3F provides cell toxicity curve for Compound-A49 against Ncl-H 522 cell line.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. Like numbers refer to like elements throughout. The present invention is described fully herein with non-limiting embodiments and exemplary experimentation.

For purposes of determining infringement, the scope of the present invention encompasses any one or more of the appended claims, including equivalents of the substance, elements or limitations as described herein.

The present invention thus provides a compound of Formula (A):

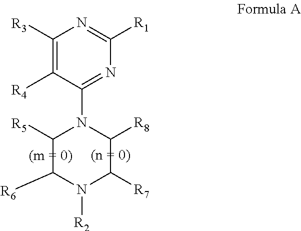

Wherein
R1, R2, R3, R4, R5, R6, R7 and R8 is selected from the group consisting of formulae provided in Table 1 to create various derivative of the parent molecule Formula A; and
m=0 and n=0.

In a first embodiment, the present invention thus includes compounds in accordance with Table 2:

TABLE 2
List of derivatives of Formula A

| | 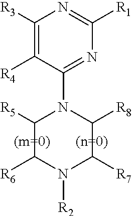 m = 0, and n = 0 Formula A | | | | | | | | |
|---|---|---|---|---|---|---|---|---|---|
| Compound | R Groups | | | | | | | | |
| No. | 1 | 2 | 3 | 4 | 5 | 6 | 7 | 8 | IUPAC |
| A1 | 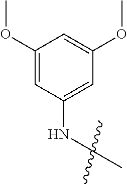 [SMILES: *Nc1cc(OC)cc(OC)c1] | 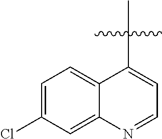 [SMILES: *c1ccnc2cc(Cl)ccc12] | CF₃ | H | H | H | H | H | 4-(4-(7-chloroquinolin-4- yl)piperazin-1-yl)-N-(3,5- dimethoxyphenyl)-6- (trifluoromethyl)pyrimidin- 2-amine |
| A2 | 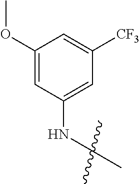 [SMILES: *Nc1cc(OC)cc(C(F)(F)F)c1] | 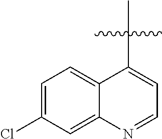 [SMILES: *c1ccnc2cc(Cl)ccc12] | CH₃ | H | H | H | H | H | 4-(4-(7-chloroquinolin-4- yl)piperazin-1-yl)-N-(3- methoxy-5- (trifluoromethyl)phenyl)- 6-methylpyrimidin-2- amine |
| A3 | 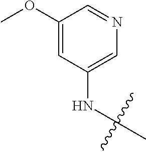 [SMILES: *Nc1cncc(OC)c1] | 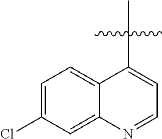 [SMILES: *c1ccnc2cc(Cl)ccc12] | CH₃ | H | H | H | H | H | 4-(4-(7-chloroquinolin-4- yl)piperazin-1-yl)-N-(5- methoxypyridin-3-yl)-6- methylpyrimidin-2-amine |
| A4 | 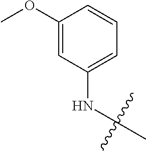 [SMILES: *Nc1cccc(OC)c1] | 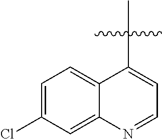 [SMILES: *c1ccnc2cc(Cl)ccc12] | CH₃ | H | H | H | H | H | 4-(4-(7-chloroquinolin-4- yl)piperazin-1-yl)-N-(3- methoxyphenyl)-6- methylpyrimidin-2-amine |
| A5 | 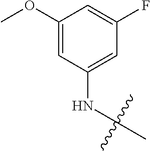 [SMILES: *Nc1cc(F)cc(OC)c1] | 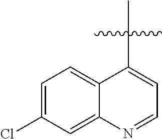 [SMILES: *c1ccnc2cc(Cl)ccc12] | CH₃ | H | H | H | H | H | 4-(4-(7-chloroquinolin-4- yl)piperazin-1-yl)-N-(3- fluoro-5-methoxyphenyl)- 6-methylpyrimidin-2- amine |
| A6 | 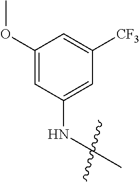 [SMILES: *Nc1cc(OC)cc(C(F)(F)F)c1] | 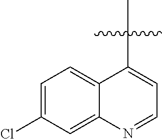 [SMILES: *c1ccnc2cc(Cl)ccc12] | CF₃ | H | H | H | H | H | 4-(4-(7-chloroquinolin-4- yl)piperazin-1-yl)-N-(3- methoxy-5- (trifluoromethyl)phenyl)- 6- (trifluoromethyl)pyrimidin- 2-amine |
| A7 | 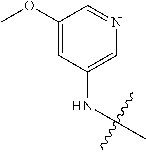 [SMILES: *Nc1cncc(OC)c1] | 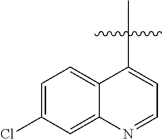 [SMILES: *c1ccnc2cc(Cl)ccc12] | CF₃ | H | H | H | H | H | 4-(4-(7-chloroquinolin-4- yl)piperazin-1-yl)-N-(5- methoxypyridin-3-yl)-6- (trifluoromethyl)pyrimidin- 2-amine |
| A8 | 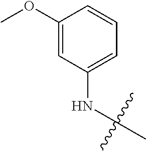 [SMILES: *Nc1cccc(OC)c1] | 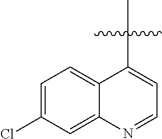 [SMILES: *c1ccnc2cc(Cl)ccc12] | CF₃ | H | H | H | H | H | 4-(4-(7-chloroquinolin-4- yl)piperazin-1-yl)-N-(3- methoxyphenyl)-6- (trifluoromethyl)pyrimidin- 2-amine |
| A9 | 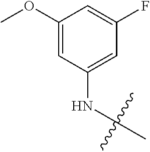 [SMILES: *Nc1cc(F)cc(OC)c1] | 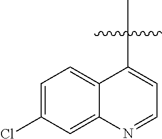 [SMILES: *c1ccnc2cc(Cl)ccc12] | CF₃ | H | H | H | H | H | 4-(4-(7-chloroquinolin-4- yl)piperazin-1-yl)-N-(3- fluoro-5-methoxyphenyl)- 6- (trifluoromethyl)pyrimidin- 2-amine |
| A10 | 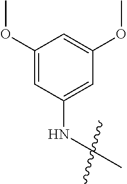 [SMILES: *Nc1cc(OC)cc(OC)c1] | 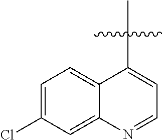 [SMILES: *c1ccnc2cc(Cl)ccc12] | CH₃ | H | H | H | H | H | 4-(4-(7-chloroquinolin-4- yl)piperazin-1-yl)-N-(3,5- dimethoxyphenyl)-6- methylpyridin-2-amine |
| A11 | 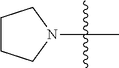 [SMILES: *N1CCCC1] | 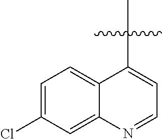 [SMILES: *c1ccnc2cc(Cl)ccc12] | CH₃ | H | H | H | H | H | 7-chloro-4-(4-(6-methyl- 2-(pyrrolidin-1- yl)pyrimidin-4- yl)piperazin-1- yl)quinoline |
| A12 | 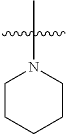 [SMILES: *N1CCCCC1] | 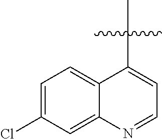 [SMILES: *c1ccnc2cc(Cl)ccc12] | CH₃ | H | H | H | H | H | 7-chloro-4-(4-(6-methyl- 2-(piperidin-1- yl)pyrimidin-4- yl)piperazin-1- yl)quinoline |
| A13 | 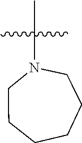 [SMILES: *N1CCCCCC1] | 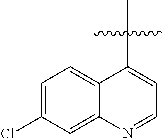 [SMILES: *c1ccnc2cc(Cl)ccc12] | CH₃ | H | H | H | H | H | 4-(4-(2-(azepan-1-yl)-6- methylpyrimidin-4- yl)piperazin-1-yl)-7- chloroquinoline |
| A14 | 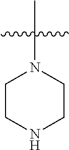 [SMILES: *N1CCNCC1] | 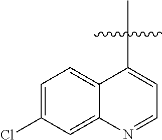 [SMILES: *c1ccnc2cc(Cl)ccc12] | CH₃ | H | H | H | H | H | 7-chloro-4-(4-(6-methyl- 2-(piperazin-1- yl)pyrimidin-4- yl)piperazin-1- yl)quinoline |
| A15 | 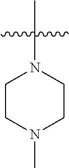 [SMILES: *N1CCN(C)CC1] | 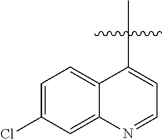 [SMILES: *c1ccnc2cc(Cl)ccc12] | CH₃ | H | H | H | H | H | 7-chloro-4-(4-(6-methyl- 2-(4-methylpiperazin-1- yl)pyrimidin-4- yl)piperazin-1- yl)quinoline |
| A16 | 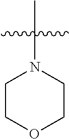 [SMILES: *N1CCOCC1] | 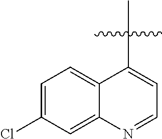 [SMILES: *c1ccnc2cc(Cl)ccc12] | CH₃ | H | H | H | H | H | 4-(4-(4-(7-chloroquinolin- 4-yl)piperazin-1-yl)-6- methylpyrimidin-2- yl)morpholine |
| A17 | 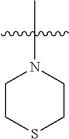 [SMILES: *N1CCSCC1] | 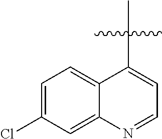 [SMILES: *c1ccnc2cc(Cl)ccc12] | CH₃ | H | H | H | H | H | 4-(4-(4-(7-chloroquinolin- 4-yl)piperazin-1-yl)-6- methylpyrimidin-2- yl)thiomorpholine |
| A18 | 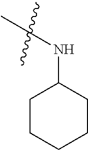 [SMILES: *NC1CCCCC1] | 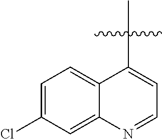 [SMILES: *c1ccnc2cc(Cl)ccc12] | CH₃ | H | H | H | H | H | 4-(4-(7-chloroquinolin-4- yl)piperazin-1-yl)-N- cyclohexyl-6- methylpyrimidin-2-amine |
| A19 | 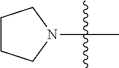 [SMILES: *N1CCCC1] | 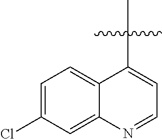 [SMILES: *c1ccnc2cc(Cl)ccc12] | CF₃ | H | H | H | H | H | 7-chloro-4-(4-(2- (pyrrolidin-1-yl)-6- (trifluoromethyl)pyrimidin- 4-yl)piperazin-1- yl)quinoline |
| A20 | 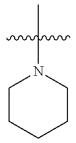 [SMILES: *N1CCCCC1] | 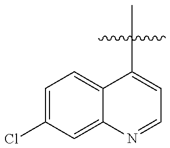 [SMILES: *c1ccnc2cc(Cl)ccc12] | CF₃ | H | H | H | H | H | 7-chloro-4-(4-(2- (piperidin-1-yl)-6- (trifluoromethyl)pyrimidin- 4-yl)piperazin-1- yl)quinoline |
| A21 | 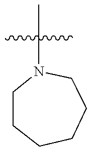 [SMILES: *N1CCCCCC1] | 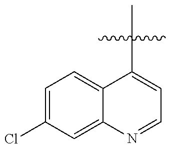 [SMILES: *c1ccnc2cc(Cl)ccc12] | CF₃ | H | H | H | H | H | 4-(4-(2-(azepan-1-yl)-6- (trifluoromethyl)pyrimidin- 4-yl)piperazin-1-yl)-7- chloroquinoline |
| A22 | 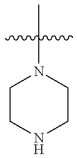 [SMILES: *N1CCNCC1] | 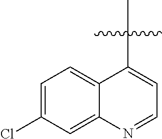 [SMILES: *c1ccnc2cc(Cl)ccc12] | CF₃ | H | H | H | H | H | 7-chloro-4-(4-(2- (piperazin-1-yl)-6- (trifluoromethyl)pyrimidin- 4-yl)piperazin-1- yl)quinoline |
| A23 | 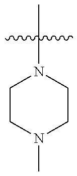 [SMILES: *N1CCN(C)CC1] | 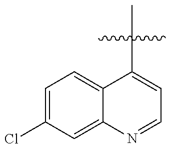 [SMILES: *c1ccnc2cc(Cl)ccc12] | CF₃ | H | H | H | H | H | 7-chloro-4-(4-(2-(4- methylpiperazin-1-yl)-6- (trifluoromethyl)pyrimidin- 4-yl)piperazin-1- yl)quinoline |
| A24 | 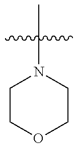 [SMILES: *N1CCOCC1] | 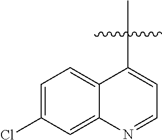 [SMILES: *c1ccnc2cc(Cl)ccc12] | CF₃ | H | H | H | H | H | 4-(4-(4-(7-chloroquinolin- 4-yl)piperazin-1-yl)-6- (trifluoromethyl)pyrimidin- 2-yl)morpholine |
| A25 | 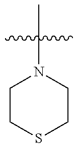 [SMILES: *N1CCSCC1] | 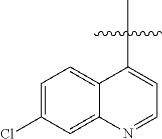 [SMILES: *c1ccnc2cc(Cl)ccc12] | CF₃ | H | H | H | H | H | 4-(4-(4-(7-chloroquinolin- 4-yl)piperazin-1-yl)-6- (trifluoromethyl)pyrimidin- 2-yl)thiomorpholine |
| A26 | 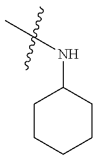 [SMILES: *NC1CCCCC1] | 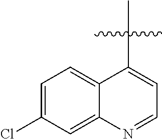 [SMILES: *c1ccnc2cc(Cl)ccc12] | CF₃ | H | H | H | H | H | 4-(4-(7-chloroquinolin-4- yl)piperazin-1-yl)-N- cyclohexyl-6- (trifluoromethyl)pyrimidin- 2-amine |
| A27 | 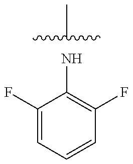 | 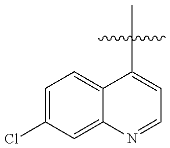 [SMILES: *c1ccnc2cc(Cl)ccc12] | CH₃ | H | H | H | H | H | 4-(4-(7-chloroquinolin-4- yl)piperazin-1-yl)-N-(2,6- difluorophenyl)-6- methylpyrimidin-2-amine |
| A28 | 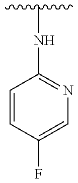 [SMILES: *Nc1ccc(F)cn1] | 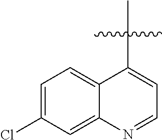 [SMILES: *c1ccnc2cc(Cl)ccc12] | CH₃ | H | H | H | H | H | 4-(4-(7-chloroquinolin-4- yl)piperazin-1-yl)-N-(5- fluoropyridin-2-yl)-6- methylpyrimidin-2-amine |
| A29 | 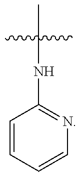 [SMILES: *Nc1ccccn1] | 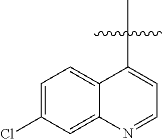 [SMILES: *c1ccnc2cc(Cl)ccc12] | CH₃ | H | H | H | H | H | 4-(4-(7-chloroquinolin-4- yl)piperazin-1-yl)-6- methyl-N-(pyridin-2- yl)pyrimidin-2-amine |
| A30 | 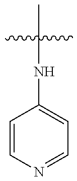 [SMILES: *Nc1ccncc1] | 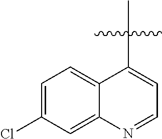 [SMILES: *c1ccnc2cc(Cl)ccc12] | CH₃ | H | H | H | H | H | 4-(4-(7-chloroquinolin-4- yl)piperazin-1-yl)-6- methyl-N-(pyridin-4- yl)pyrimidin-2-amine |
| A31 | 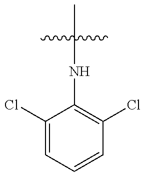 [SMILES: *Nc1c(Cl)cccc1Cl] | 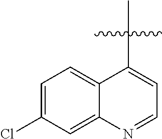 [SMILES: *c1ccnc2cc(Cl)ccc12] | CH₃ | H | H | H | H | H | 4-(4-(7-chloroquinolin-4- yl)piperazin-1-yl)-N-(2,6- dichlorophenyl)-6- methylpyrimidin-2-amine |
| A32 | 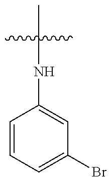 [SMILES: *Nc1cccc(Br)c1] | 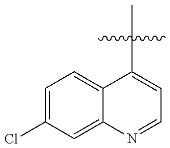 [SMILES: *c1ccnc2cc(Cl)ccc12] | CH₃ | H | H | H | H | H | N-(3-bromophenyl)-4-(4- (7-chloroquinolin-4- yl)piperazin-1-yl)-6- methylpyrimidin-2-amine |
| A33 | 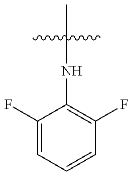 [SMILES: *Nc1c(F)cccc1F] | 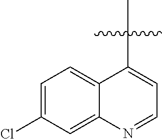 [SMILES: *c1ccnc2cc(Cl)ccc12] | CF₃ | H | H | H | H | H | 4-(4-(7-chloroquinolin-4- yl)piperazin-1-yl)-N-(2,6- difluorophenyl)-6- (trifluoromethyl)pyrimidin- 2-amine |
| A34 | 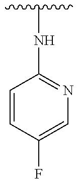 [SMILES: *Nc1ccc(F)cn1] | 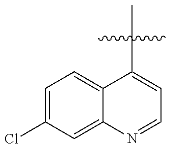 [SMILES: *c1ccnc2cc(Cl)ccc12] | CF₃ | H | H | H | H | H | 4-(4-(7-chloroquinolin-4- yl)piperazin-1-yl)-N-(5- fluoropyridin-2-yl)-6- (trifluoromethyl)pyrimidin- 2-amine |
| A35 | 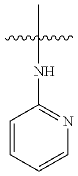 [SMILES: *Nc1ccccn1] | 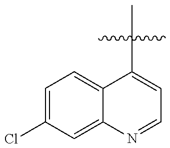 [SMILES: *c1ccnc2cc(Cl)ccc12] | CF₃ | H | H | H | H | H | 4-(4-(7-chloroquinolin-4- yl)piperazin-1-yl)-N- (pyridin-2-yl)-6- (trifluoromethyl)pyrimidin- 2-amine |
| A36 | 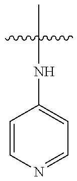 [SMILES: *Nc1ccncc1] | 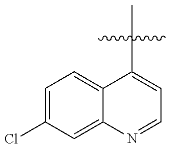 [SMILES: *c1ccnc2cc(Cl)ccc12] | CF₃ | H | H | H | H | H | 4-(4-(7-chloroquinolin-4- yl)piperazin-1-yl)-N- (pyridin-4-yl)-6- (trifluoromethyl)pyrimidin- 2-amine |
| A37 | 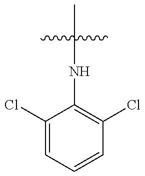 [SMILES: *Nc1c(Cl)cccc1Cl] | 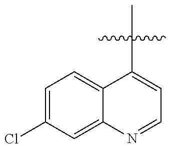 [SMILES: *c1ccnc2cc(Cl)ccc12] | CF₃ | H | H | H | H | H | 4-(4-(7-chloroquinolin-4- yl)piperazin-1-yl)-N-(2,3- dichlorophenyl)-6- (trifluoromethyl)pyrimidin- 2-amine |
| A38 | 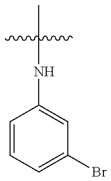 [SMILES: *Nc1cccc(Br)c1] | 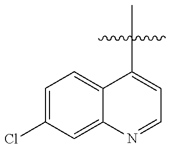 [SMILES: *c1ccnc2cc(Cl)ccc12] | CF₃ | H | H | H | H | H | N-(3-bromophenyl)-4-(4- (7-chloroquinolin-4- yl)piperazin-1-yl)-6- (trifluoromethyl)pyrimidin- 2-amine |
| A39 | 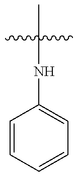 [SMILES: *Nc1ccccc1] | 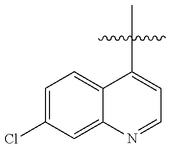 [SMILES: *c1ccnc2cc(Cl)ccc12] | CH₃ | H | H | H | H | H | 4-(4-(7-chloroquinolin-4- yl)piperazin-1-yl)-6- methyl-N- phenylpyrimidin-2-amine |
| A40 | 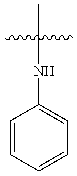 [SMILES: *Nc1ccccc1] | 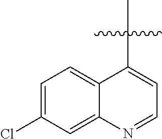 [SMILES: *c1ccnc2cc(Cl)ccc12] | CF₃ | H | H | H | H | H | 4-(4-(7-chloroquinolin-4- yl)piperazin-1-yl)-N- phenyl-6- (trifluoromethyl)pyrimidin- 2-amine |
| A41 | 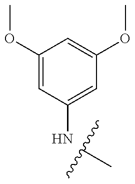 | 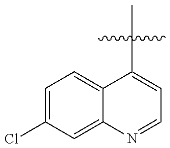 [SMILES: *c1ccnc2cc(Cl)ccc12] | H | H | H | H | H | H | 4-(4-(7-chloroquinolin-4- yl)piperazin-1-yl)-N-(3,5- dimethoxyphenyl)pyrimid in-2-amine |
| A42 | 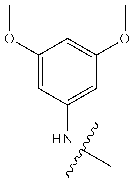 | 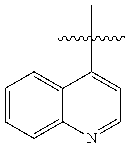 [SMILES: *c1ccnc2ccccc12] | CH | H | H | H | H | H | N-(3,5- dimethoxyphenyl)-4- methyl-6-(4-(quinolin-4- yl)piperazin-1- yl)pyrimidin-2-amine |
| A43 | 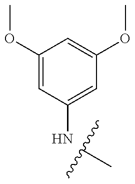 | 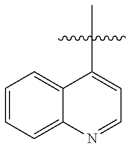 [SMILES: *c1ccnc2ccccc12] | CF3 | H | H | H | H | H | N-(3,5- dimethoxyphenyl)-4-(4- (quinolin-4-yl)piperazin- 1-yl)-6- (trifluoromethyl)pyrimidi n-2-amine |
| A44 | 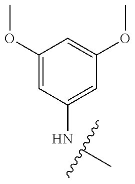 | 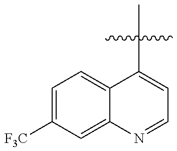 [SMILES: *c1ccnc2cc(C(F)(F)F)ccc12] | CH | H | H | H | H | H | N-(3,5- dimethoxyphenyl)-4- methyl-6-(4-(7- (trifluoromethyl)quinolin- 4-yl)piperazin-1- yl)pyrimidin-2-amine |
| A45 | 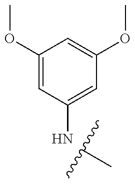 | 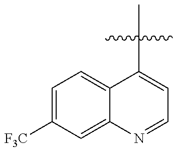 [SMILES: *c1ccnc2cc(C(F)(F)F)ccc12] | CF | H | H | H | H | H | N-(3,5- dimethoxyphenyl)-4- (trifluoromethyl)-6-(4-(7- (trifluoromethyl)quinolin- 4-yl)piperazin-1- yl)pyrimidin-2-amine |
| A46 | 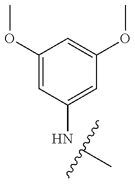 | 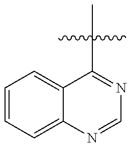 [SMILES: *c1ncnc2ccccc12] | CH₃ | H | H | H | H | H | N-(3,5- dimethoxyphenyl)-4- methyl-6-(4-(quinazolin- 4-yl)piperazin-1- yl)pyrimidin-2-amine |
| A47 | 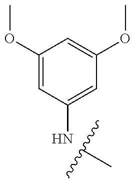 | 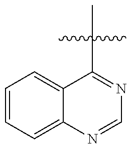 [SMILES: *c1ncnc2ccccc12] | CF₃ | H | H | H | H | H | N-(3,5- dimethoxyphenyl)-4-(4- (quinazolin-4- yl)piperazin-1-yl)-6- (trifluoromethyl)pyrimidin- 2-amine |
| A48 | 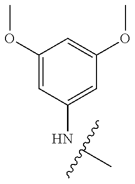 | 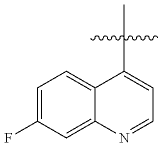 [SMILES: *c1ccnc2cc(F)ccc12] | CH₃ | H | H | H | H | H | N-(3,5- dimethoxyphenyl)-4-(4- (7-fluoroquinolin-4- yl)piperazin-1-yl)-6- methylpyrimidin-2-amine |
| A49 | 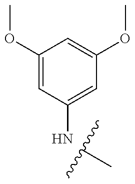 | 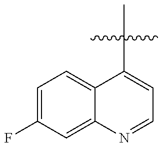 [SMILES: *c1ccnc2cc(F)ccc12] | CF₃ | H | H | H | H | H | N-(3,5- dimethoxyphenyl)-4-(4- (7-fluoroquinolin-4- yl)piperazin-1-yl)-6- (trifluoromethyl)pyrimidin- 2-amine |
| A50 | 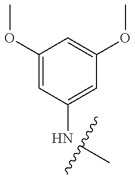 | 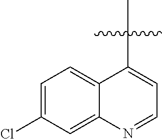 [SMILES: *c1ccnc2cc(Cl)ccc12] | CF₃ | H | H | H | H | H | 4-(4-(7-chloroquinolin-4- yl)piperazin-1-yl)-N-(3,5- dimethoxyphenyl)-6- (trifluoromethyl)pyridin- 2-amine |
| A51 | 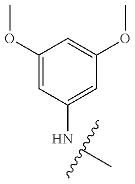 | 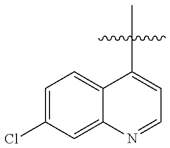 [SMILES: *c1ccnc2cc(Cl)ccc12] | CH₃ | H | H | H | H | H | 4-(4-(7-chloroquinolin-4- yl)piperazin-1-yl)-N-(3,5- dimethoxyphenyl)-6- methylpyridin-2-amine |
| A52 | 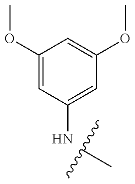 | 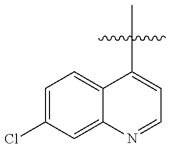 [SMILES: *c1ccnc2cc(Cl)ccc12] | F | H | H | H | H | H | 4-(4-(7-chloroquinolin-4- yl)piperazin-1-yl)-N-(3,5- dimethoxyphenyl)-6- fluoropyrimidin-2-amine |
| A53 | 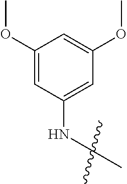 [SMILES: *Nc1cc(OC)cc(OC)c1] | 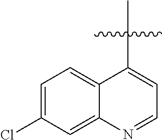 [SMILES: *c1ccnc2cc(Cl)ccc12] | CH₃ | F | H | H | H | H | 4-(4-(7-chloroquinolin-4- yl)piperazin-1-yl)-N-(3,5- dimethoxyphenyl)-5- fluoro-6- methylpyrimidin-2-amine |
| A54 | 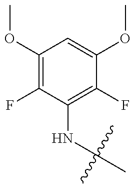 [SMILES: *Nc1c(F)c(OC)cc(OC)c1F] | 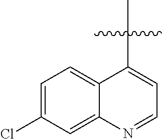 [SMILES: *c1ccnc2cc(Cl)ccc12] | CH₃ | H | H | H | H | H | 4-(4-(7-chloroquinolin-4- yl)piperazin-1-yl)-N-(2,6- difluoro-3,5- dimethoxyphenyl)-6- methylpyrimidin-2-amine |
| A55 | 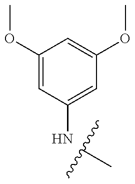 | 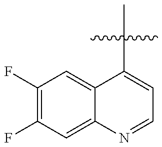 [SMILES: *c1ccnc2cc(F)c(F)cc12] | CH₃ | H | H | H | H | H | 4-(4-(6,7- difluoroquinolin-4- yl)piperazin-1-yl)-N-(3,5- dimethoxyphenyl)-6- methylpyrimidin-2-amine |
| A56 | 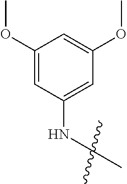 [SMILES: *Nc1cc(OC)cc(OC)c1] | 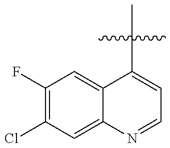 [SMILES: *c1ccnc2cc(Cl)c(F)cc12] | CH₃ | H | H | H | H | H | 4-(4-(7-chloro-6- fluoroquinolin-4- yl)piperazin-1-yl)-N-(3,5- dimethoxyphenyl)-6- methylpyrimidin-2-amine |
| A57 | 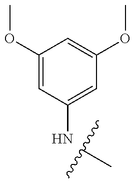 | 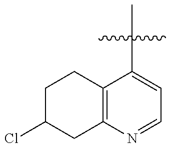 [SMILES: *c1ccnc2c1CCC(Cl)C2] | CH₃ | H | H | H | H | H | 4-(4-(7-chloro- 4a,5,6,7,8,8a- hexahydroquinolin-4- yl)piperazin-1-yl)-N-(3,5- dimethoxyphenyl)-6- methylpyrimidin-2-amine |
| A58 | 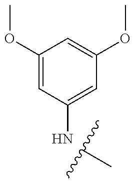 | 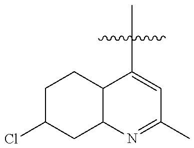 [SMILES: *C1=CC(C)=NC2CC(Cl)CCC12] | CH₃ | H | H | H | H | H | 4-(4-(7-chloro-2-methyl- 4a,5,6,7,8,8a- hexahydroquinolin-4- yl)piperazin-1-yl)-N-(3,5- dimethoxyphenyl)-6- methylpyrimidin-2-amine |
| A59 | 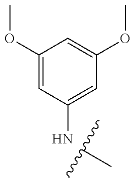 | 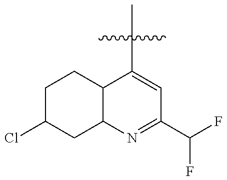 [SMILES: *C1=CC(C(F)F)=NC2CC(Cl)CCC12] | CH₃ | H | H | H | H | H | 4-(4-(7-chloro-2- (difluoromethyl)- 4a,5,6,7,8,8a- hexahydroquinolin-4- yl)piperazin-1-yl)-N-(3,5- dimethoxyphenyl)-6- methylpyrimidin-2-amine |
| A60 | 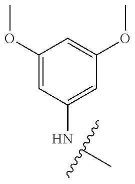 | 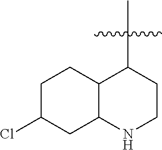 [SMILES: *C1CCNC2CC(Cl)CCC12] | CH₃ | H | H | H | H | H | 4-(4-(decahydroquinolin- 4-yl)piperazin-1-yl)-N- (3,5-dimethoxyphenyl)-6- methylpyrimidin-2-amine |
| A61 | 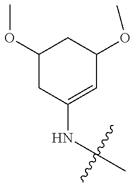 [SMILES: *NC1=CC(OC)CC(OC)C1] | 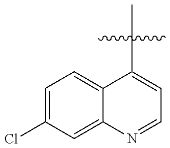 [SMILES: *c1ccnc2cc(Cl)ccc12] | CH₃ | H | H | H | H | H | 4-(4-(7-chloroquinolin-4- yl)piperazin-1-yl)-N-(3,5- dimethoxycyclohex-1-en- 1-yl)-6-methylpyrimidin- 2-amine |
| A62 | 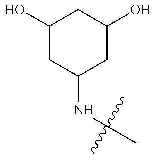 [SMILES: *NC1CC(O)CC(O)C1] | 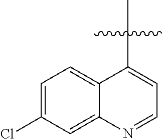 [SMILES: *c1ccnc2cc(Cl)ccc12] | CH₃ | H | H | H | H | H | 5-((4-(4-(7- chloroquinolin-4- yl)piperazin-1-yl)-6- methylpyrimidin-2- yl)amino)cyclohexane- 1,3-diol |
| A63 | 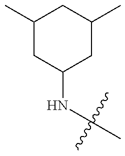 | 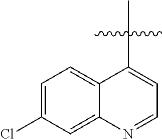 [SMILES: *c1ccnc2cc(Cl)ccc12] | CH₃ | H | H | H | H | H | 4-(4-(7-chloroquinolin-4- yl)piperazin-1-yl)-N-(3,5- dimethylcyclohexyl)-6- methylpyrimidin-2-amine |
| A64 | 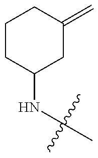 [SMILES: *NC1CCCC(=C)C1] | 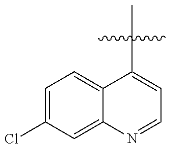 [SMILES: *c1ccnc2cc(Cl)ccc12] | CH₃ | H | H | H | H | H | 4-(4-(7-chloroquinolin-4- yl)piperazin-1-yl)-6- methyl-N-(3- methylenecyclohexyl) pyrimidin-2-amine |
| A65 | 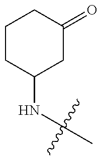 [SMILES: *NC1CCCC(=O)C1] | 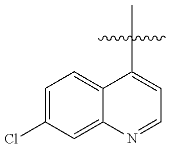 [SMILES: *c1ccnc2cc(Cl)ccc12] | CH₃ | H | H | H | H | H | 3-((4-(4-(7- chloroquinolin-4- yl)piperazin-1-yl)-6- methylpyrimidin-2- yl)amino)cyclohexanone |
| A66 | 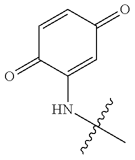 [SMILES: *NC1=CC(=O)C=CC1=O] | 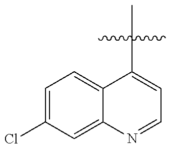 [SMILES: *c1ccnc2cc(Cl)ccc12] | CH₃ | H | H | H | H | H | 2-((4-(4-(7- chloroquinolin-4- yl)piperazin-1-yl)-6- methylpyrimidin-2- yl)amino)cyclohexa-2,5- diene-1,4-dione |
| A67 | 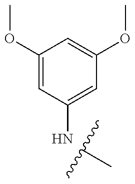 | 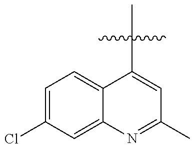 [SMILES: *c1cc(C)nc2cc(Cl)ccc12] | CH₃ | H | H | H | H | H | 4-(4-(7-chloro-2- methylquinolin-4- yl)piperazin-1-yl)-N-(3,5- dimethoxyphenyl)-6- methylpyrimidin-2-amine |
| A68 | 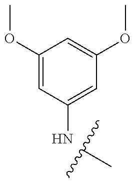 | 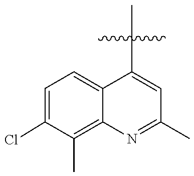 [SMILES: *c1cc(C)nc2c(C)c(Cl)ccc12] | CH₃ | H | H | H | H | H | 4-(4-(7-chloro-2,8- dimethylquinolin-4- yl)piperazin-1-yl)-N-(3,5- dimethoxyphenyl)-6- methylpyrimidin-2-amine |
| A69 | 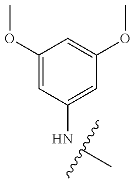 | 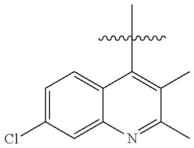 [SMILES: *c1c(C)c(C)nc2cc(Cl)ccc12] | CH₃ | H | H | H | H | H | 4-(4-(7-chloro-2,3- dimethylquinolin-4- yl)piperazin-1-yl)-N-(3,5- dimethoxyphenyl)-6- methylpyrimidin-2-amine |
| A70 | 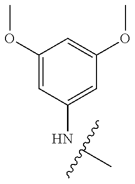 | 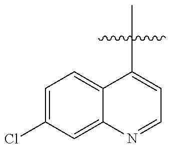 [SMILES: *c1ccnc2cc(Cl)ccc12] | CH₃ | H | CH₃ | H | H | CH₃ | 4-(4-(7-chloroquinolin-4- yl)-2,6- dimethylpiperazin-1-yl)- N-(3,5- dimethoxyphenyl)-6- methylpyrimidin-2-amine |
| A71 | 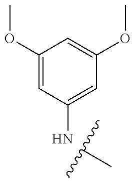 | 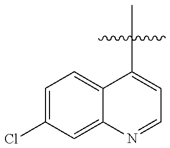 [SMILES: *c1ccnc2cc(Cl)ccc12] | CH₃ | H | H | H | H | 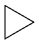 | 4-(7-(7-chloroquinolin-4- yl)-4,7- diazaspiro[2.5]octan-4- yl)-N-(3,5- dimethoxyphenyl)-6- methylpyrimidin-2-amine |
| A72 | 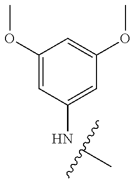 | 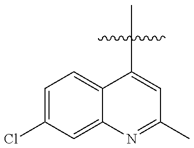 [SMILES: *c1cc(C)nc2cc(Cl)ccc12] | CH₃ | H | H | H | H | CH₃ | 4-(4-(7-chloro-2- methylquinolin-4-yl)-2- methylpiperazin-1-yl)-N- (3,5-dimethoxyphenyl)-6- methylpyrimidin-2-amine |
| A73 | 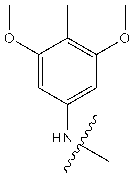 | 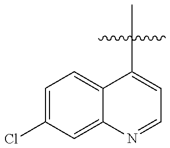 [SMILES: *c1ccnc2cc(Cl)ccc12] | CH₃ | CH₃ | H | H | H | H | 4-(4-(7-chloroquinolin-4- yl)piperazin-1-yl)-N-(3,5- dimethoxy-4- methylphenyl)-5,6- dimethylpyrimidin-2- amine |
| A74 | 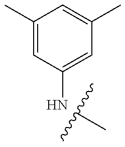 | 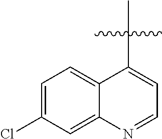 [SMILES: *c1ccnc2cc(Cl)ccc12] | CH₃ | CH₃ | H | H | H | H | 4-(4-(7-chloroquinolin-4- yl)piperazin-1-yl)-N-(3,5- dimethylphenyl)-5,6- dimethylpyrimidin-2- amine |
| A75 | 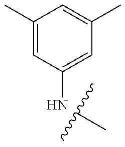 | 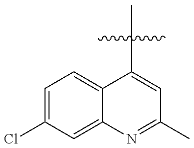 [SMILES: *c1cc(C)nc2cc(Cl)ccc12] | CH₃ | CH₃ | H | H | CH₃ | H | 4-(4-(7-chloro-2- methylquinolin-4-yl)-3- methylpiperazin-1-yl)-N- (3,5-dimethylphenyl)-5,6- dimethylpyrimidin-2- amine |
| A76 | 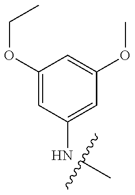 | 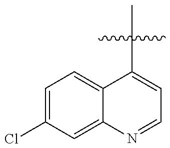 [SMILES: *c1ccnc2cc(Cl)ccc12] | CH₃ | H | H | H | H | H | 4-(4-(7-chloroquinolin-4- yl)piperazin-1-yl)-N-(3- ethoxy-5- methoxyphenyl)-6- methylpyrimidin-2-amine |
| A77 | 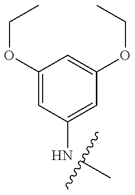 | 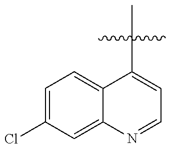 [SMILES: *c1ccnc2cc(Cl)ccc12] | CH₃ | H | H | H | H | H | 4-(4-(7-chloroquinolin-4- yl)piperazin-1-yl)-N-(3,5- diethoxyphenyl)-6- methylpyrimidin-2-amine |
| A78 | 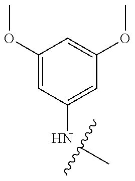 | 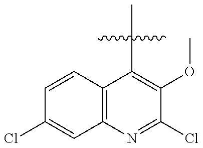 [SMILES: *c1c(OC)c(Cl)nc2cc(Cl)ccc12] | CH₃ | H | H | H | H | H | 4-(4-(2,7-dichloro-3- methoxyquinolin-4- yl)piperazin-1-yl)-N-(3,5- dimethoxyphenyl)-6- methylpyrimidin-2-amine |
| A79 | 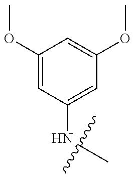 | 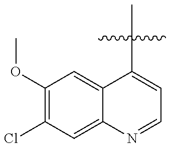 [SMILES: *c1ccnc2cc(Cl)c(OC)cc12] | CH₃ | H | H | H | H | H | 4-(4-(7-chloro-6- methoxyquinolin-4- yl)piperazin-1-yl)-N-(3,5- dimethoxyphenyl)-6- methylpyrimidin-2-amine |
| A80 | 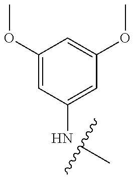 | 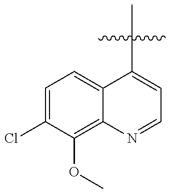 [SMILES: *c1ccnc2c(OC)c(Cl)ccc12] | CH₃ | H | H | H | H | H | 4-(4-(7-chloro-8- methoxyquinolin-4- yl)piperazin-1-yl)-N-(3,5- dimethoxyphenyl)-6- methylpyrimidin-2-amine |
| A81 | 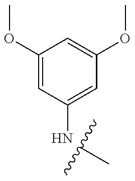 | 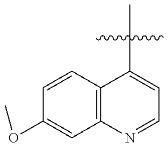 [SMILES: *c1ccnc2cc(OC)ccc12] | CH₃ | H | H | H | H | H | N-(3,5- dimethoxyphenyl)-4-(4- (7-methoxyquinolin-4- yl)piperazin-1-yl)-6- methylpyrimidin-2-amine |
| A82 | 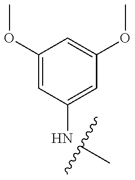 | 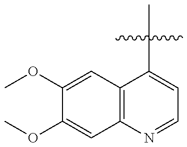 [SMILES: *c1ccnc2cc(OC)c(OC)cc12] | CH₃ | H | H | H | H | H | N-(3,5- dimethoxyphenyl)-4-(4- (6,7-dimethoxyquinolin- 4-yl)piperazin-1-yl)-6- methylpyrimidin-2-amine |
| A83 | 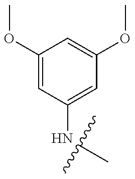 | 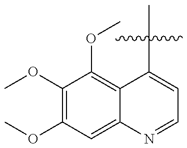 [SMILES: *c1ccnc2cc(OC)c(OC)c(OC)c12] | CH₃ | H | H | H | H | H | N-(3,5- dimethoxyphenyl)-4- methyl-6-(4-(5,6,7- trimethoxyquinolin-4- yl)piperazin-1- yl)pyrimidin-2-amine |
| A84 | 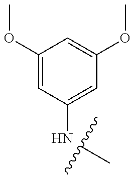 | 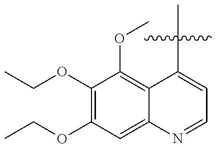 [SMILES: *c1ccnc2cc(OCC)c(OCC)c(OC)c12] | CH₃ | H | H | H | H | H | 4-(4-(6,7-diethoxy-5- methoxyquinolin-4- yl)piperazin-1-yl)-N-(3,5- dimethoxyphenyl)-6- methylpyrimidin-2-amine |
| A85 | 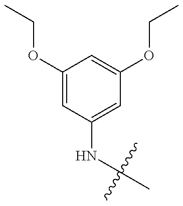 [SMILES: *Nc1cc(OCC)cc(OCC)c1] | 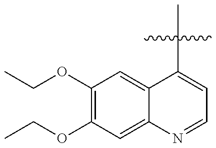 [SMILES: *c1ccnc2cc(OCC)c(OCC)cc12] | CH₃ | H | H | H | H | H | N-(3,5-diethoxyphenyl)- 4-(4-(6,7- diethoxyquinolin-4- yl)piperazin-1-yl)-6- methylpyrimidin-2-amine |
| A86 | 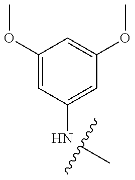 | 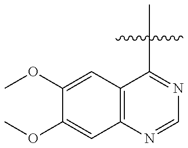 [SMILES: *c1ncnc2cc(OC)c(OC)cc12] | CH₃ | H | H | H | H | H | N-(3,5- dimethoxyphenyl)-4-(4- (6,7- dimethoxyquinazolin-4- yl)piperazin-1-yl)-6- methylpyrimidin-2-amine |
| A87 | 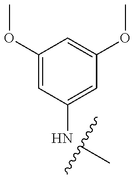 | 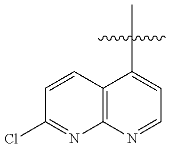 [SMILES: *c1ccnc2nc(Cl)ccc12] | CH₃ | H | H | H | H | H | 4-(4-(7-chloro-1,5- naphthyridin-4- yl)piperazin-1-yl)-N-(3,5- dimethoxyphenyl)-6- methylpyrimidin-2-amine |
| A88 | 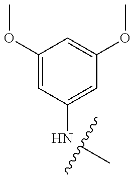 | 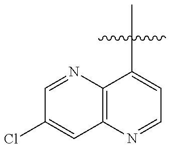 [SMILES: *c1ccnc2cc(Cl)cnc12] | CH₃ | H | H | H | H | H | 4-(4-(7-chloro-1,5- naphthyridin-4- yl)piperazin-1-yl)-N-(3,5- dimethoxyphenyl)-6- methylpyrimidin-2-amine |
| A89 | 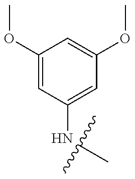 | 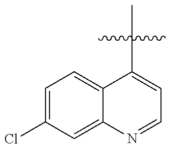 [SMILES: *c1ccnc2cc(Cl)ccc12] | CH₃ | H | H | H | H | — | 4-(3-(7-chloroquinolin-4- yl)imidazolidin-1-yl)-N- (3,5-dimethoxyphenyl)-6- methylpyrimidin-2-amine |
| A90 | 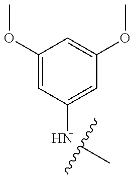 | 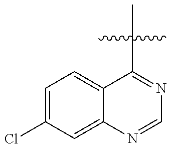 [SMILES: *c1ncnc2cc(Cl)ccc12] | CH₃ | H | H | H | H | — | 4-(3-(7-chloroquinazolin- 4-yl)imidazolidin-1-yl)- N-(3,5- dimethoxyphenyl)-6- methylpyrimidin-2-amine |
| A91 | 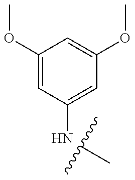 | 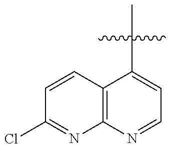 [SMILES: *c1ccnc2nc(Cl)ccc12] | CH₃ | H | H | H | H | — | 4-(3-(7-chloro-1,8- naphthyridin-4- yl)imidazolidin-1-yl)-N- (3,5-dimethoxyphenyl)-6- methylpyrimidin-2-amine |
| A92 | 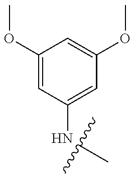 | 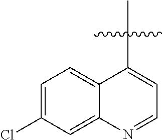 [SMILES: *c1ccnc2cc(Cl)ccc12] | CH₃ | H | H | H | H | H | 4-(4-(7-chloroquinolin-4- yl)-1,4-diazepan-1-yl)-N- (3,5-dimethoxyphenyl)-6- methylpyrimidin-2-amine |
| A93 | 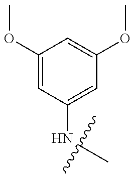 | 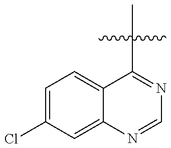 [SMILES: *c1ncnc2cc(Cl)ccc12] | CH₃ | H | H | H | H | H | 4-(4-(7-chloroquinolin-4- yl)-1,4-diazepan-1-yl)-N- (3,5-dimethoxyphenyl)-6- methylpyrimidin-2-amine |
| A94 | 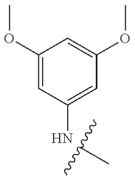 | 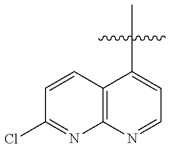 [SMILES: *c1ccnc2nc(Cl)ccc12] | CH₃ | H | H | H | H | H | 4-(4-(7-chloro-1,8- naphthyridin-4-yl)-1,4- diazepan-1-yl)-N-(3,5- dimethoxyphenyl)-6- methylpyrimidin-2-amine |
| A95 | 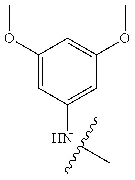 | 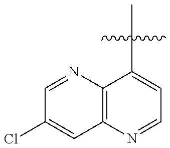 [SMILES: *c1ccnc2cc(Cl)cnc12] | CH₃ | H | H | H | H | H | 4-(4-(7-chloro-1,5- naphthyridin-4-yl)-1,4- diazepan-1-yl)-N-(3,5- dimethoxyphenyl)-6- methylpyrimidin-2-amine |
| A96 | 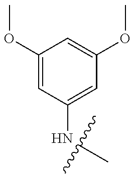 | 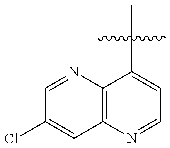 [SMILES: *c1ccnc2cc(Cl)cnc12] | CF₃ | H | H | H | H | H | 4-(4-(7-chloro-1,5- naphthyridin-4-yl)-1,4- diazepan-1-yl)-N-(3,5- dimethoxyphenyl)-6- (trifluoromethyl)pyrimidin- 2-amine |
| A97 | 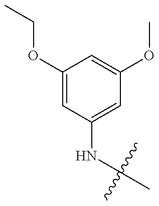 [SMILES: *Nc1cc(OC)cc(OCC)c1] | 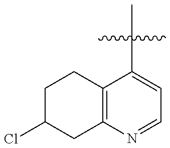 [SMILES: *c1ccnc2c1CCC(Cl)C2] | F | H | H | H | H | H | 4-(4-(7-chloro- 4a,5,6,7,8,8a- hexahydroquinolin-4- yl)piperazin-1-yl)-N-(3- ethoxy-5- methoxyphenyl)-6- fluoropyrimidin-2-amine |
| A98 | 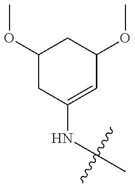 [SMILES: *NC1=CC(OC)CC(OC)C1] | 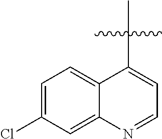 [SMILES: *c1ccnc2cc(Cl)ccc12] | CF₃ | H | H | H | H | 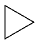 | 4-(7-(7-chloroquinolin-4- yl)-4,7- diazaspiro[2.5]octan-4- yl)-N-(3,5- dimethoxycyclohex-1-en- 1-yl)-6- (trifluoromethyl)pyrimidin- 2-amine |
| A99 | 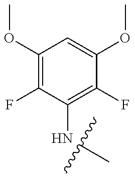 | 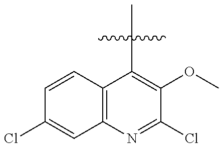 [SMILES: *c1c(OC)c(Cl)nc2cc(Cl)ccc12] | CH₃ | H | H | H | H | H | 4-(4-(2,7-dichloro-3- methoxyquinolin-4- yl)piperazin-1-yl)-N-(2,6- difluoro-3,5- dimethoxyphenyl)-6- methylpyrimidin-2-amine |
| A100 | 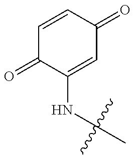 [SMILES: *NC1=CC(=O)C=CC1=O] | 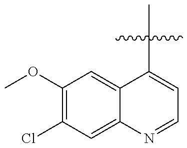 [SMILES: *c1ccnc2cc(Cl)c(OC)cc12] | CH₃ | CH₃ | H | H | H | CH₃ | N-(3,5-diethoxyphenyl)- 4-(3-(6,7- dimethoxyquinazolin-4- yl)imidazolidin-1-yl)-6- methylpyrimidin-2-amine |
| A101 | 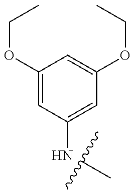 | 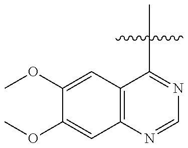 [SMILES: *c1ncnc2cc(OC)c(OC)cc12] | CH₃ | H | H | H | H | — | N-(3,5-diethoxyphenyl)- 4-(3-(6,7- dimethoxyquinazolin-4- yl)imidazolidin-1-yl)-6- methylpyrimidin-2-amine |
| A102 | 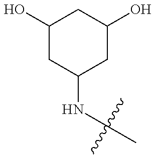 [SMILES: *NC1CC(O)CC(O)C1] | 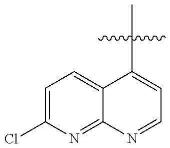 [SMILES: *c1ccnc2nc(Cl)ccc12] | CH₃ | H | H | O | 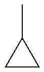 | — | 3-(7-chloro-1,8- naphthyridin-4-yl)-2- cyclopropyl-1-(2-((3,5- dihydroxycyclohexyl) amino)-6-methylpyrimidin-4- yl)imidazolidin-4-one |
| A103 | 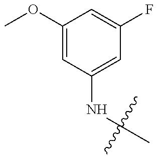 [SMILES: *Nc1cc(F)cc(OC)c1] | 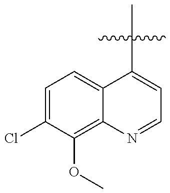 [SMILES: *c1ccnc2c(OC)c(Cl)ccc12] | CH₃ | H | H | H | H | — | 4-(3-(7-chloro-8- methoxyquinolin-4- yl)imidazolidin-1-yl)-N- (3-fluoro-5- methoxyphenyl)-6- methylpyrimidin-2-amine |
| A104 | 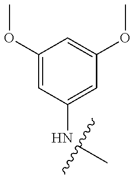 | 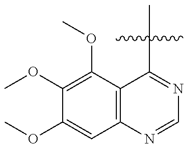 [SMILES: *c1ncnc2cc(OC)c(OC)c(OC)c12] | CH₃ | CH₃ | H | H | H | H | N-(3,5- dimethoxyphenyl)-4,5- dimethyl-6-(4-(5,6,7- trimethoxyquinazolin-4- yl)-1,4-diazepan-1- yl)pyrimidin-2-amine |
| A105 | 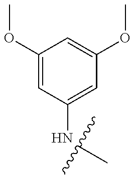 | 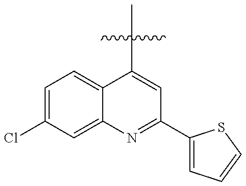 [SMILES: *c1cc(-c2cccs2)nc2cc(Cl)ccc12] | CH₃ | H | H | H | H | H | 4-(4-(7-chloro-2- (thiophen-2-yl)-1,5- naphthyridin-4-yl)-1,4- diazepan-1-yl)-N-(3,5- dimethoxyphenyl)-6- methylpyrimidin-2-amine |
| A106 | 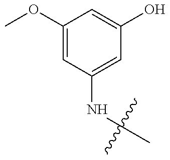 [SMILES: *Nc1cc(O)cc(OC)c1] | 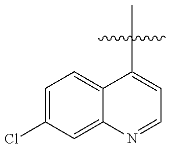 [SMILES: *c1ccnc2cc(Cl)ccc12] | CH₃ | H | H | H | H | H | 3-((4-(4-(7- chloroquinolin-4- yl)piperazin-1-yl)-6- methylpyrimidin-2- yl)amino)-5- methoxyphenol |
| A107 | 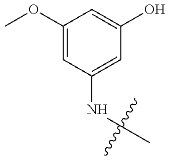 [SMILES: *Nc1cc(O)cc(OC)c1] | 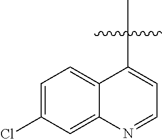 [SMILES: *c1ccnc2cc(Cl)ccc12] | CF₃ | H | H | H | H | H | 3-((4-(4-(7- chloroquinolin-4- yl)piperazin-1-yl)-6- (trifluoromethyl)pyrimidin- 2-yl)amino)-5- methoxyphenol |
| A108 | 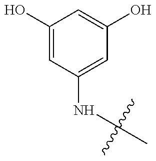 [SMILES: *Nc1cc(O)cc(O)c1] | 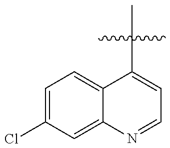 [SMILES: *c1ccnc2cc(Cl)ccc12] | CH₃ | H | H | H | H | H | 5-((4-(4-(7- chloroquinolin-4- yl)piperazin-1-yl)-6- methylpyrimidin-2- yl)amino)benzene-1,3- diol |
| A109 | 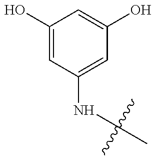 [SMILES: *Nc1cc(O)cc(O)c1] | 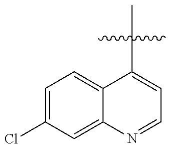 [SMILES: *c1ccnc2cc(Cl)ccc12] | CF₃ | H | H | H | H | H | 5-((4-(4-(7- chloroquinolin-4- yl)piperazin-1-yl)-6- (trifluoromethyl)pyrimidin- 2-yl)amino)benzene- 1,3-diol |
| A110 | 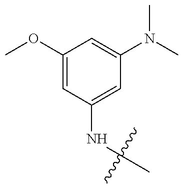 | 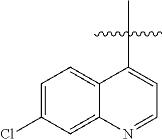 [SMILES: *c1ccnc2cc(Cl)ccc12] | CH | H | H | H | H | H | N1-(4-(4-(7- chloroquinolin-4- yl)piperazin-1-yl)-6- methylpyrimidin-2-yl)-5- methoxy-N3,N3- dimethylbenzene-1,3- diamine |
| A111 | 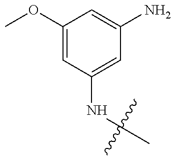 [SMILES: *Nc1cc(N)cc(OC)c1] | 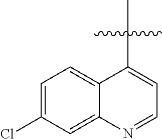 [SMILES: *c1ccnc2cc(Cl)ccc12] | CH₃ | H | H | H | H | H | N1-(4-(4-(7- chloroquinolin-4- yl)piperazin-1-yl)-6- methylpyrimidin-2-yl)-5- methoxybenzene-1,3- diamine |
| A112 | 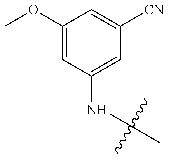 [SMILES: *Nc1cc(C#N)cc(OC)c1] | 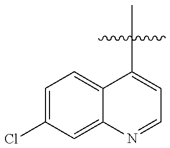 [SMILES: *c1ccnc2cc(Cl)ccc12] | CH₃ | H | H | H | H | H | 3-((4-(4-(7- chloroquinolin-4- yl)piperazin-1-yl)-6- methylpyrimidin-2- yl)amino)-5- methoxybenzonitrile |
| A113 | 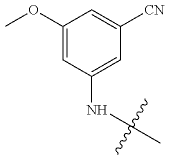 [SMILES: *Nc1cc(C#N)cc(OC)c1] | 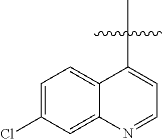 [SMILES: *c1ccnc2cc(Cl)ccc12] | CF₃ | H | H | H | H | H | 3-((4-(4-(7- chloroquinolin-4- yl)piperazin-1-yl)-6- (trifluoromethyl)pyrimidin- 2-yl)amino)-5- methoxybenzonitrile |
| A114 | 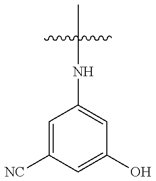 [SMILES: *Nc1cc(O)cc(C#N)c1] | 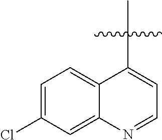 [SMILES: *c1ccnc2cc(Cl)ccc12] | CF₃ | H | H | H | H | H | 3-((4-(4-(7- chloroquinolin-4- yl)piperazin-1-yl)-6- (trifluoromethyl)pyrimidin- 2-yl)amino)-5- hydroxybenzonitrile |
| A115 | 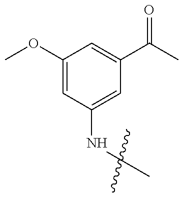 [SMILES: *Nc1cc(OC)cc(C(C)=O)c1] | 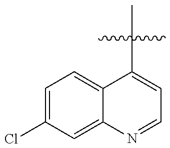 [SMILES: *c1ccnc2cc(Cl)ccc12] | CH₃ | H | H | H | H | H | 1-(3-((4-(4-(7- chloroquinolin-4- yl)piperazin-1-yl)-6- methylpyrimidin-2- yl)amino)-5- methoxyphenyl)ethanone |
| A116 | 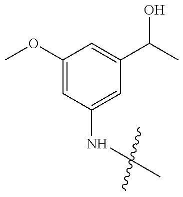 [SMILES: *Nc1cc(OC)cc(C(C)O)c1] | 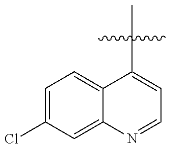 [SMILES: *c1ccnc2cc(Cl)ccc12] | CH₃ | H | H | H | H | H | 1-(3-((4-(4-(7- chloroquinolin-4- yl)piperazin-1-yl)-6- methylpyrimidin-2- yl)amino)-5- methoxyphenyl)ethanol |

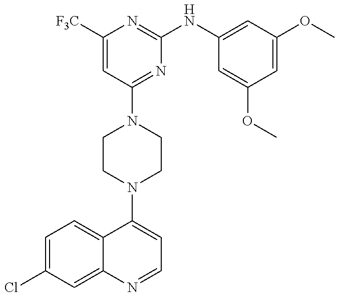 [SMILES: COc1cc(Nc2nc(N3CCN(c4ccnc5cc(Cl)ccc45)CC3)cc(C(F)(F)F)n2)cc(OC)c1]

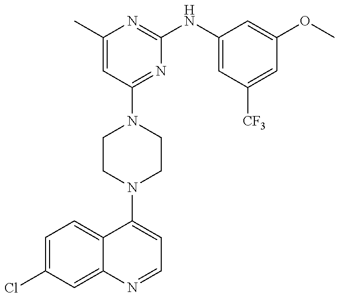 [SMILES: COc1cc(Nc2nc(C)cc(N3CCN(c4ccnc5cc(Cl)ccc45)CC3)n2)cc(C(F)(F)F)c1]

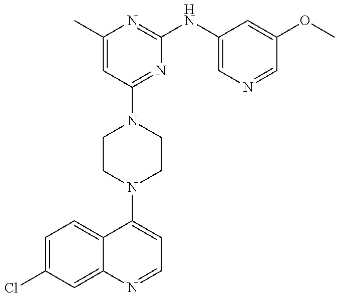 [SMILES: COc1cncc(Nc2nc(C)cc(N3CCN(c4ccnc5cc(Cl)ccc45)CC3)n2)c1]

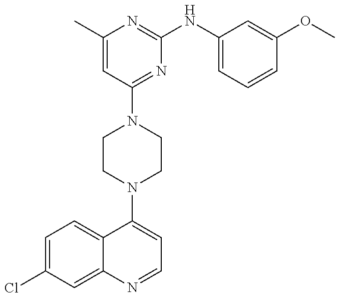 [SMILES: COc1cccc(Nc2nc(C)cc(N3CCN(c4ccnc5cc(Cl)ccc45)CC3)n2)c1]

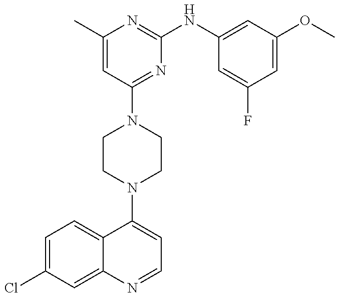 [SMILES: COc1cc(F)cc(Nc2nc(C)cc(N3CCN(c4ccnc5cc(Cl)ccc45)CC3)n2)c1]

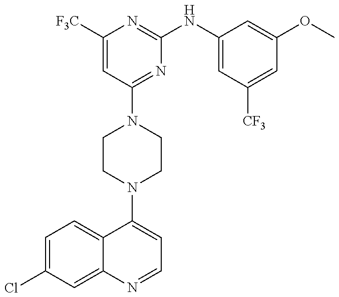 [SMILES: COc1cc(Nc2nc(N3CCN(c4ccnc5cc(Cl)ccc45)CC3)cc(C(F)(F)F)n2)cc(C(F)(F)F)c1]

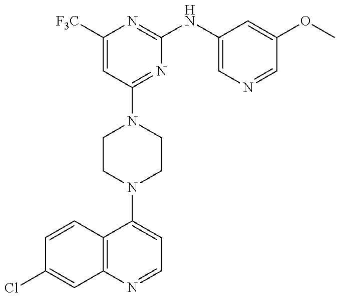 [SMILES: COc1cncc(Nc2nc(N3CCN(c4ccnc5cc(Cl)ccc45)CC3)cc(C(F)(F)F)n2)c1]

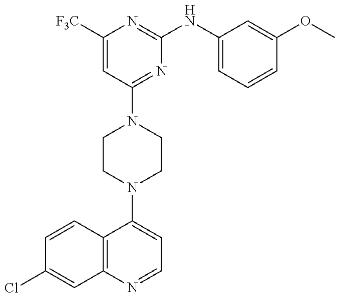 [SMILES: COc1cccc(Nc2nc(N3CCN(c4ccnc5cc(Cl)ccc45)CC3)cc(C(F)(F)F)n2)c1]

Compound-A1
Compound-A2
Compound-A3
Compound-A4
Compound-A5
Compound-A6
Compound-A7
Compound-A8
Compound-A9
Compound-A10
Compound-A11
Compound-A12 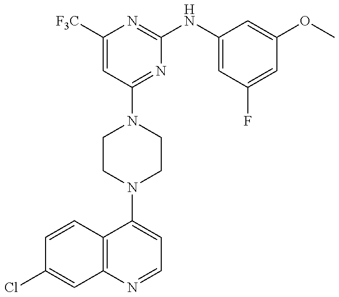 [SMILES: COc1cc(F)cc(Nc2nc(N3CCN(c4ccnc5cc(Cl)ccc45)CC3)cc(C(F)(F)F)n2)c1] 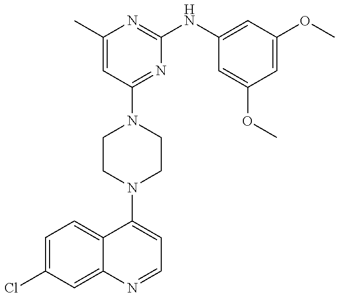 [SMILES: COc1cc(Nc2nc(C)cc(N3CCN(c4ccnc5cc(Cl)ccc45)CC3)n2)cc(OC)c1] 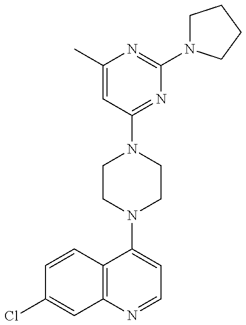 [SMILES: Cc1cc(N2CCN(c3ccnc4cc(Cl)ccc34)CC2)nc(N2CCCC2)n1] 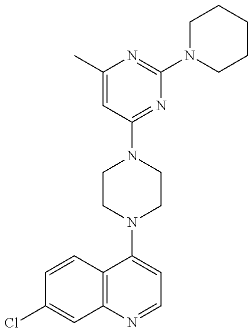 [SMILES: Cc1cc(N2CCN(c3ccnc4cc(Cl)ccc34)CC2)nc(N2CCCCC2)n1] 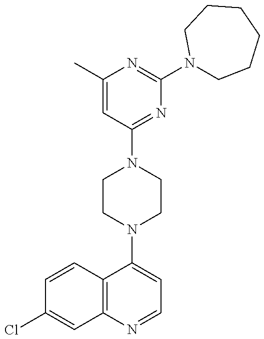 [SMILES: Cc1cc(N2CCN(c3ccnc4cc(Cl)ccc34)CC2)nc(N2CCCCCC2)n1]
Compound-A13 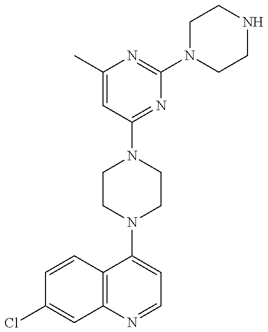 [SMILES: Cc1cc(N2CCN(c3ccnc4cc(Cl)ccc34)CC2)nc(N2CCNCC2)n1]
Compound-A14 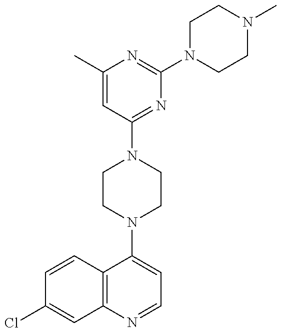 [SMILES: Cc1cc(N2CCN(c3ccnc4cc(Cl)ccc34)CC2)nc(N2CCN(C)CC2)n1]
Compound-A15 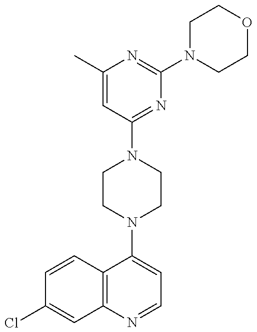 [SMILES: Cc1cc(N2CCN(c3ccnc4cc(Cl)ccc34)CC2)nc(N2CCOCC2)n1]
Compound-A16 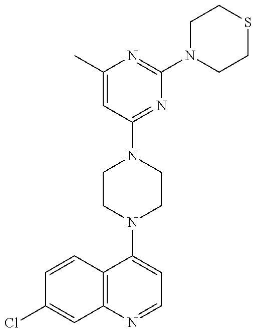 [SMILES: Cc1cc(N2CCN(c3ccnc4cc(Cl)ccc34)CC2)nc(N2CCSCC2)n1]
Compound-A17 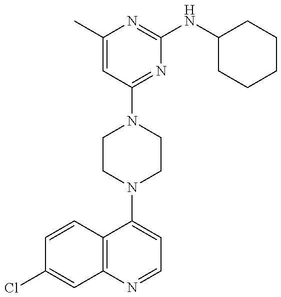 [SMILES: Cc1cc(N2CCN(c3ccnc4cc(Cl)ccc34)CC2)nc(NC2CCCCC2)n1]
Compound-A18 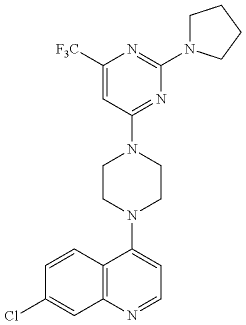 [SMILES: FC(F)(F)c1cc(N2CCN(c3ccnc4cc(Cl)ccc34)CC2)nc(N2CCCC2)n1]
Compound-A19 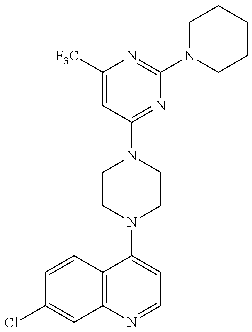 [SMILES: FC(F)(F)c1cc(N2CCN(c3ccnc4cc(Cl)ccc34)CC2)nc(N2CCCCC2)n1]
Compound-A20 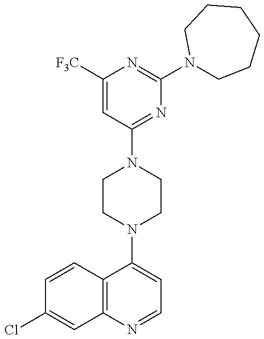 [SMILES: FC(F)(F)c1cc(N2CCN(c3ccnc4cc(Cl)ccc34)CC2)nc(N2CCCCCC2)n1]
Compound-A21 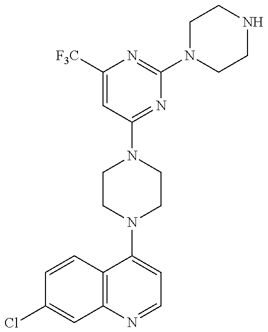 [SMILES: FC(F)(F)c1cc(N2CCN(c3ccnc4cc(Cl)ccc34)CC2)nc(N2CCNCC2)n1]
Compound-A22 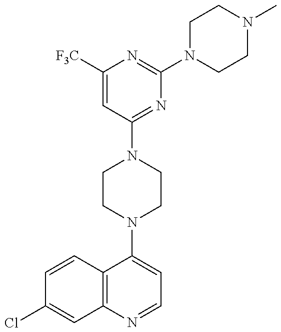 [SMILES: CN1CCN(c2nc(N3CCN(c4ccnc5cc(Cl)ccc45)CC3)cc(C(F)(F)F)n2)CC1]
Compound-A23 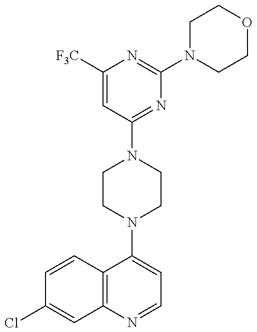 [SMILES: FC(F)(F)c1cc(N2CCN(c3ccnc4cc(Cl)ccc34)CC2)nc(N2CCOCC2)n1]
Compound-A24 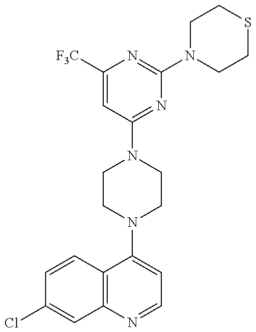 [SMILES: FC(F)(F)c1cc(N2CCN(c3ccnc4cc(Cl)ccc34)CC2)nc(N2CCSCC2)n1]
Compound-A25 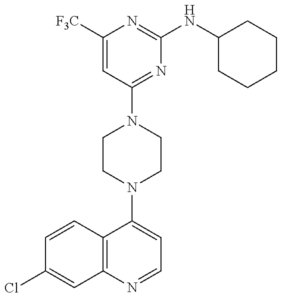 [SMILES: FC(F)(F)c1cc(N2CCN(c3ccnc4cc(Cl)ccc34)CC2)nc(NC2CCCCC2)n1]
Compound-A26 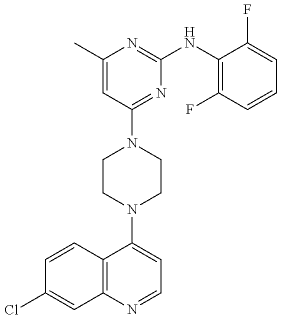 [SMILES: Cc1cc(N2CCN(c3ccnc4cc(Cl)ccc34)CC2)nc(Nc2c(F)cccc2F)n1]
Compound-A27 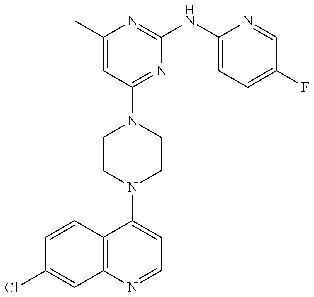 [SMILES: Cc1cc(N2CCN(c3ccnc4cc(Cl)ccc34)CC2)nc(Nc2ccc(F)cn2)n1]
Compound-A28 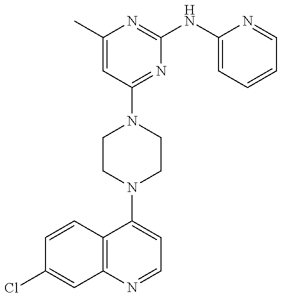 [SMILES: Cc1cc(N2CCN(c3ccnc4cc(Cl)ccc34)CC2)nc(Nc2ccccn2)n1]
Compound-A29 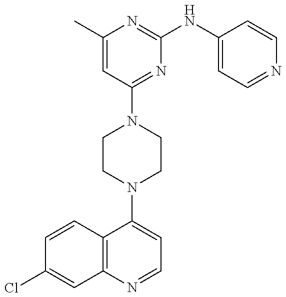 [SMILES: Cc1cc(N2CCN(c3ccnc4cc(Cl)ccc34)CC2)nc(Nc2ccncc2)n1]
Compound-A30 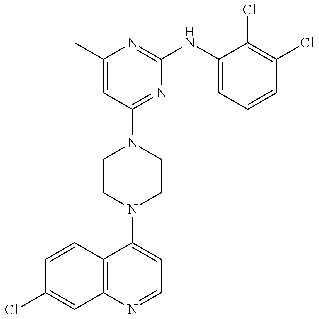 [SMILES: Cc1cc(N2CCN(c3ccnc4cc(Cl)ccc34)CC2)nc(Nc2cccc(Cl)c2Cl)n1]
Compound-A31 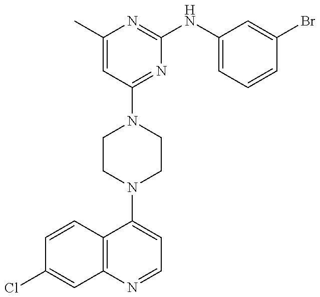 [SMILES: Cc1cc(N2CCN(c3ccnc4cc(Cl)ccc34)CC2)nc(Nc2cccc(Br)c2)n1]
Compound-A32 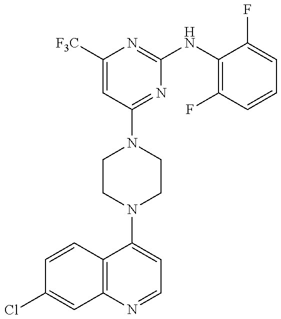 [SMILES: Fc1cccc(F)c1Nc1nc(N2CCN(c3ccnc4cc(Cl)ccc34)CC2)cc(C(F)(F)F)n1]
Compound-A33 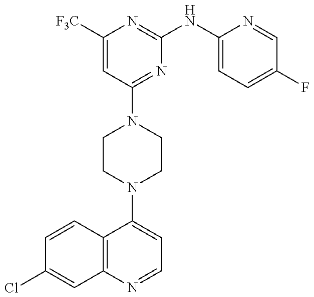 [SMILES: Fc1ccc(Nc2nc(N3CCN(c4ccnc5cc(Cl)ccc45)CC3)cc(C(F)(F)F)n2)nc1]
Compound-A34 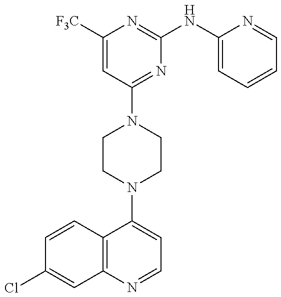 [SMILES: FC(F)(F)c1cc(N2CCN(c3ccnc4cc(Cl)ccc34)CC2)nc(Nc2ccccn2)n1]
Compound-A35 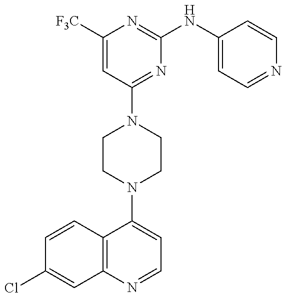 [SMILES: FC(F)(F)c1cc(N2CCN(c3ccnc4cc(Cl)ccc34)CC2)nc(Nc2ccncc2)n1]
Compound-A36 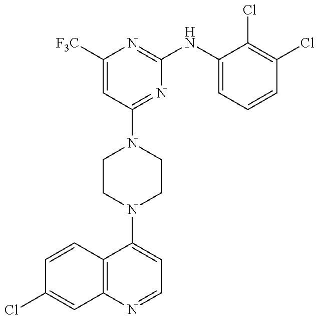 [SMILES: FC(F)(F)c1cc(N2CCN(c3ccnc4cc(Cl)ccc34)CC2)nc(Nc2cccc(Cl)c2Cl)n1]
Compound-A37 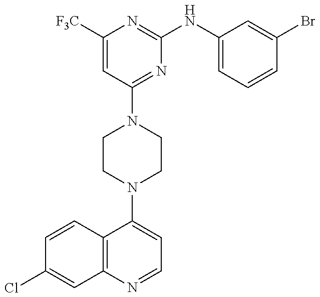 [SMILES: FC(F)(F)c1cc(N2CCN(c3ccnc4cc(Cl)ccc34)CC2)nc(Nc2cccc(Br)c2)n1]
Compound-A38 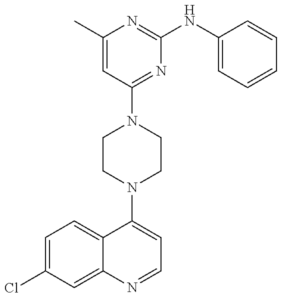 [SMILES: Cc1cc(N2CCN(c3ccnc4cc(Cl)ccc34)CC2)nc(Nc2ccccc2)n1]
Compound-A39 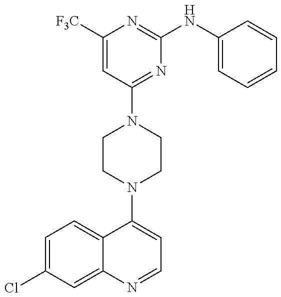 [SMILES: FC(F)(F)c1cc(N2CCN(c3ccnc4cc(Cl)ccc34)CC2)nc(Nc2ccccc2)n1]
Compound-A40 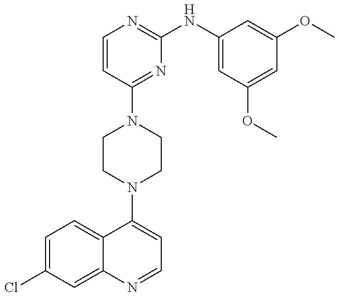 [SMILES: COc1cc(Nc2nccc(N3CCN(c4ccnc5cc(Cl)ccc45)CC3)n2)cc(OC)c1]
Compound-A41 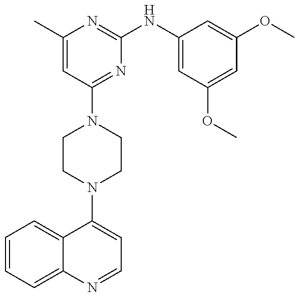 [SMILES: COc1cc(Nc2nc(C)cc(N3CCN(c4ccnc5ccccc45)CC3)n2)cc(OC)c1]
Compound-A42 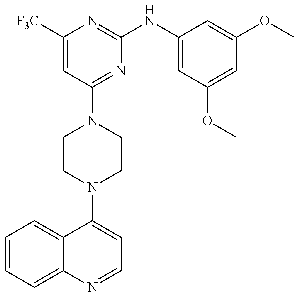 [SMILES: COc1cc(Nc2nc(N3CCN(c4ccnc5ccccc45)CC3)cc(C(F)(F)F)n2)cc(OC)c1]
Compound-A43 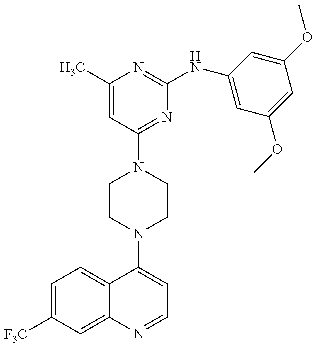 [SMILES: COc1cc(Nc2nc(C)cc(N3CCN(c4ccnc5cc(C(F)(F)F)ccc45)CC3)n2)cc(OC)c1]
Compound-A44 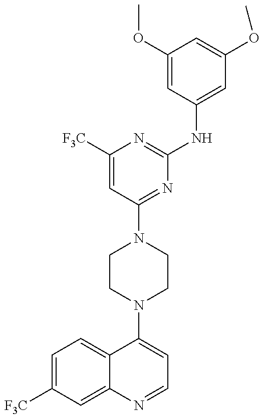 [SMILES: COc1cc(Nc2nc(N3CCN(c4ccnc5cc(C(F)(F)F)ccc45)CC3)cc(C(F)(F)F)n2)cc(OC)c1]
Compound-A45 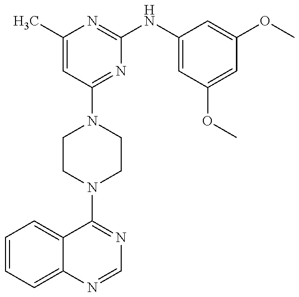 [SMILES: COc1cc(Nc2nc(C)cc(N3CCN(c4ncnc5ccccc45)CC3)n2)cc(OC)c1]
Compound-A46 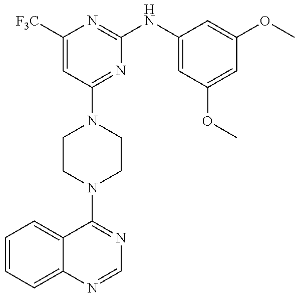 [SMILES: COc1cc(Nc2nc(N3CCN(c4ncnc5ccccc45)CC3)cc(C(F)(F)F)n2)cc(OC)c1]
Compound-A47 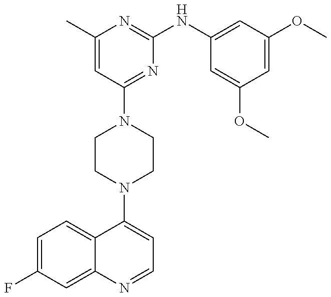 [SMILES: COc1cc(Nc2nc(C)cc(N3CCN(c4ccnc5cc(F)ccc45)CC3)n2)cc(OC)c1]
Compound-A48 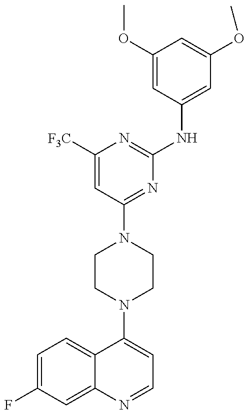 [SMILES: COc1cc(Nc2nc(N3CCN(c4ccnc5cc(F)ccc45)CC3)cc(C(F)(F)F)n2)cc(OC)c1]
Compound-A49 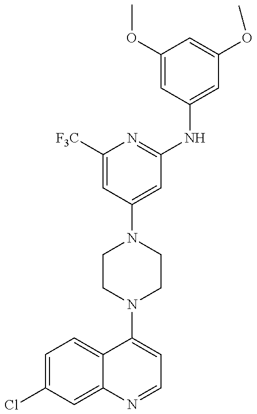 [SMILES: COc1cc(Nc2cc(N3CCN(c4ccnc5cc(Cl)ccc45)CC3)cc(C(F)(F)F)n2)cc(OC)c1]
Compound-A50 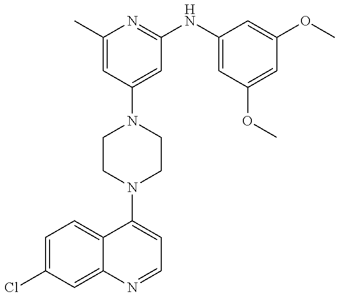 [SMILES: COc1cc(Nc2cc(N3CCN(c4ccnc5cc(Cl)ccc45)CC3)cc(C)n2)cc(OC)c1]
Compound-A51 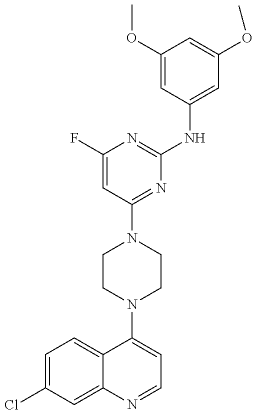 [SMILES: COc1cc(Nc2nc(F)cc(N3CCN(c4ccnc5cc(Cl)ccc45)CC3)n2)cc(OC)c1]
Compound-A52 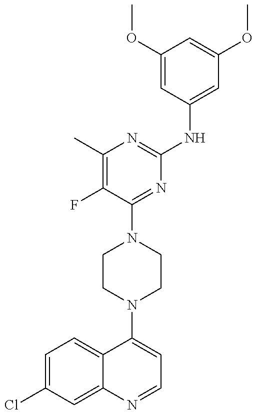 [SMILES: COc1cc(Nc2nc(C)c(F)c(N3CCN(c4ccnc5cc(Cl)ccc45)CC3)n2)cc(OC)c1]
Compound-A53 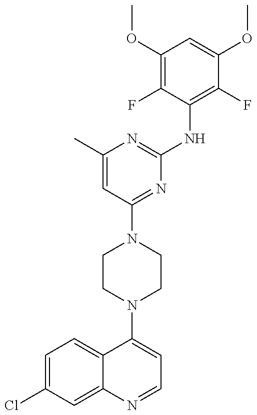 [SMILES: COc1cc(OC)c(F)c(Nc2nc(C)cc(N3CCN(c4ccnc5cc(Cl)ccc45)CC3)n2)c1F]
Compound-A54 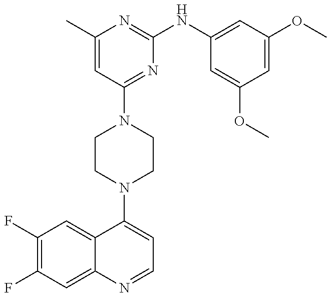 [SMILES: COc1cc(Nc2nc(C)cc(N3CCN(c4ccnc5cc(F)c(F)cc45)CC3)n2)cc(OC)c1]
Compound-A55 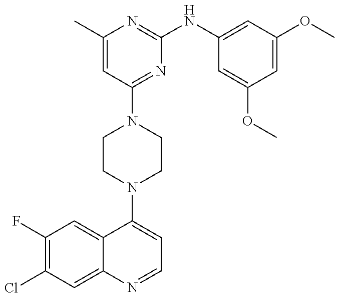 [SMILES: COc1cc(Nc2nc(C)cc(N3CCN(c4ccnc5cc(Cl)c(F)cc45)CC3)n2)cc(OC)c1]
Compound-A56 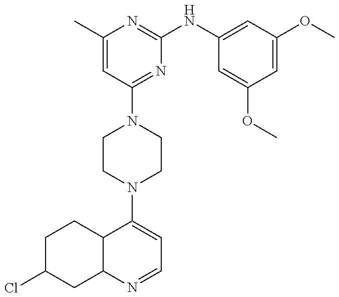 [SMILES: COc1cc(Nc2nc(C)cc(N3CCN(C4=CC=NC5CC(Cl)CCC45)CC3)n2)cc(OC)c1]
Compound-A57 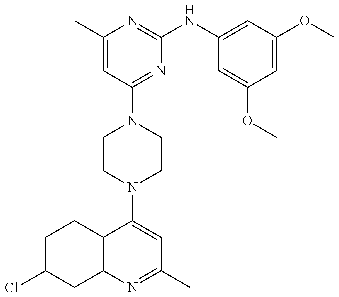 [SMILES: COc1cc(Nc2nc(C)cc(N3CCN(C4=CC(C)=NC5CC(Cl)CCC45)CC3)n2)cc(OC)c1]
Compound-A58 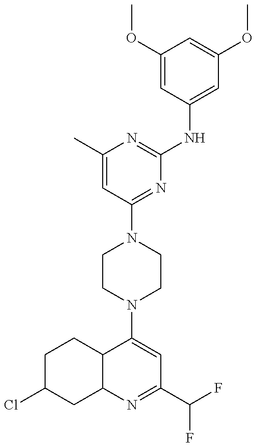 [SMILES: COc1cc(Nc2nc(C)cc(N3CCN(C4=CC(C(F)F)=NC5CC(Cl)CCC45)CC3)n2)cc(OC)c1]
Compound-A59 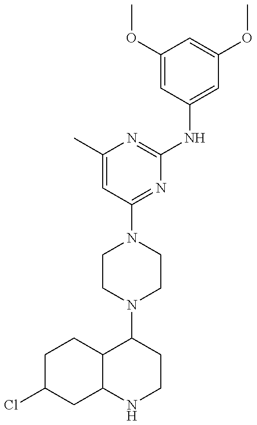 [SMILES: COc1cc(Nc2nc(C)cc(N3CCN(C4CCNC5CC(Cl)CCC54)CC3)n2)cc(OC)c1]
Compound-A60 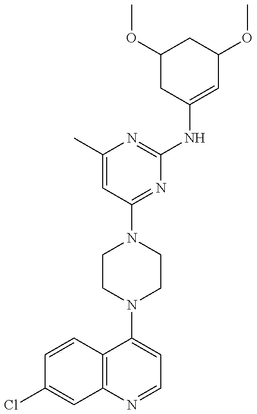 [SMILES: COC1C=C(Nc2nc(C)cc(N3CCN(c4ccnc5cc(Cl)ccc45)CC3)n2)CC(OC)C1]
Compound-A61 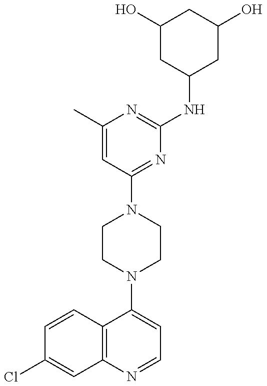 [SMILES: Cc1cc(N2CCN(c3ccnc4cc(Cl)ccc34)CC2)nc(NC2CC(O)CC(O)C2)n1]
Compound-A62 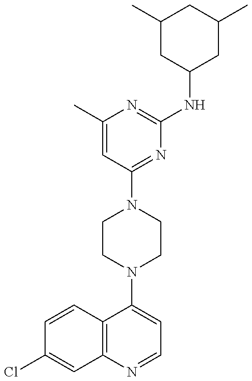 [SMILES: Cc1cc(N2CCN(c3ccnc4cc(Cl)ccc34)CC2)nc(NC2CC(C)CC(C)C2)n1]
Compound-A63 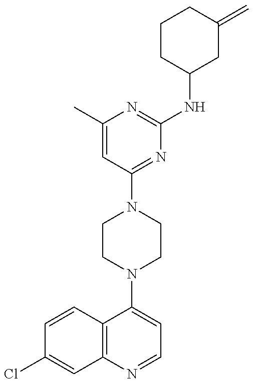 [SMILES: C=C1CCCC(Nc2nc(C)cc(N3CCN(c4ccnc5cc(Cl)ccc45)CC3)n2)C1]
Compound-A64 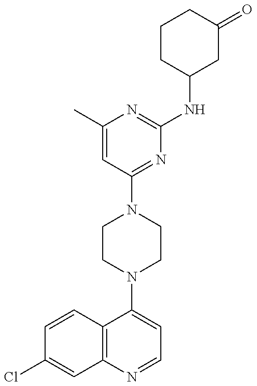 [SMILES: Cc1cc(N2CCN(c3ccnc4cc(Cl)ccc34)CC2)nc(NC2CCCC(=O)C2)n1]
Compound-A65 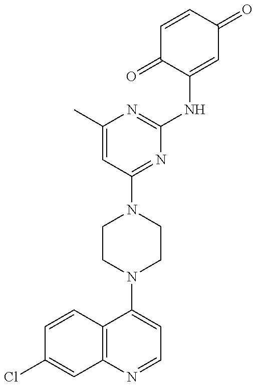 [SMILES: Cc1cc(N2CCN(c3ccnc4cc(Cl)ccc34)CC2)nc(NC2=CC(=O)C=CC2=O)n1]
Compound-A66 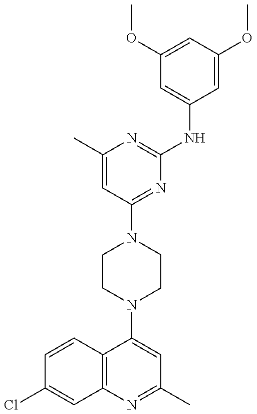 [SMILES: COc1cc(Nc2nc(C)cc(N3CCN(c4cc(C)nc5cc(Cl)ccc45)CC3)n2)cc(OC)c1]
Compound-A67 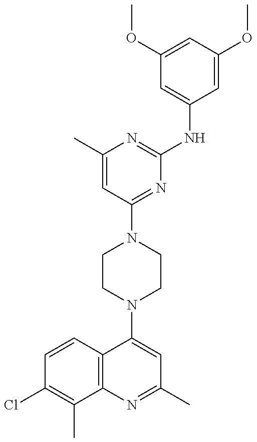 [SMILES: COc1cc(Nc2nc(C)cc(N3CCN(c4cc(C)nc5c(C)c(Cl)ccc45)CC3)n2)cc(OC)c1]
Compound-A68 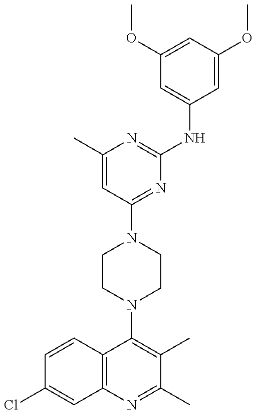 [SMILES: COc1cc(Nc2nc(C)cc(N3CCN(c4c(C)c(C)nc5cc(Cl)ccc45)CC3)n2)cc(OC)c1]
Compound-A69 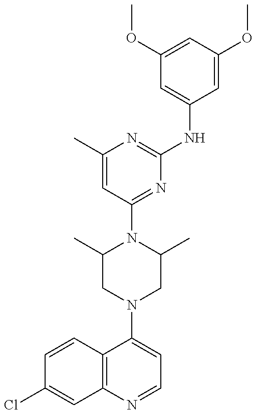 [SMILES: COc1cc(Nc2nc(C)cc(N3C(C)CN(c4ccnc5cc(Cl)ccc45)CC3C)n2)cc(OC)c1]
Compound-A70 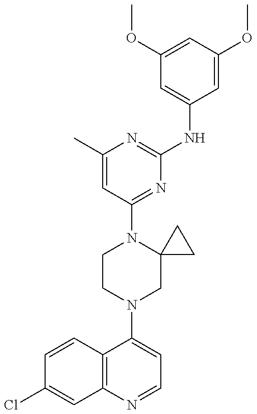 [SMILES: COc1cc(Nc2nc(C)cc(N3CCN(c4ccnc5cc(Cl)ccc45)CC34CC4)n2)cc(OC)c1]
Compound-A71 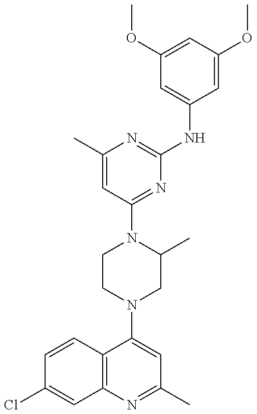 [SMILES: COc1cc(Nc2nc(C)cc(N3CCN(c4cc(C)nc5cc(Cl)ccc45)CC3C)n2)cc(OC)c1]
Compound-A72 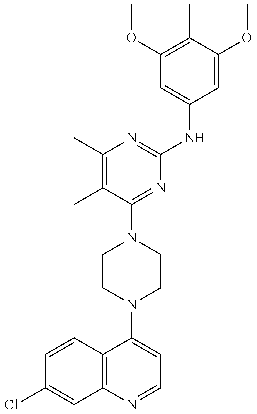 [SMILES: COc1cc(Nc2nc(C)c(C)c(N3CCN(c4ccnc5cc(Cl)ccc45)CC3)n2)cc(OC)c1C]
Compound-A73 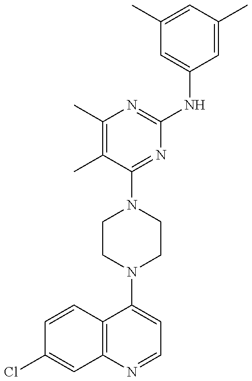 [SMILES: Cc1cc(C)cc(Nc2nc(C)c(C)c(N3CCN(c4ccnc5cc(Cl)ccc45)CC3)n2)c1]
Compound-A74 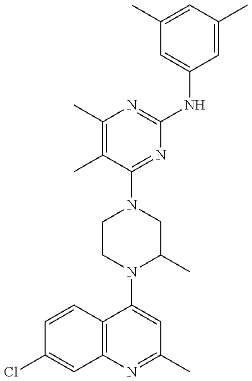 [SMILES: Cc1cc(C)cc(Nc2nc(C)c(C)c(N3CCN(c4cc(C)nc5cc(Cl)ccc45)C(C)C3)n2)c1]
Compound-A75 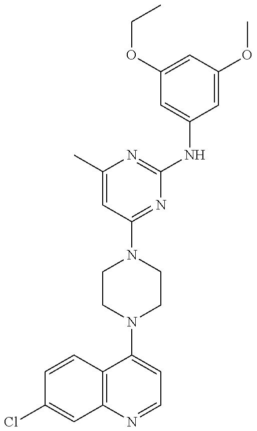 [SMILES: CCOc1cc(Nc2nc(C)cc(N3CCN(c4ccnc5cc(Cl)ccc45)CC3)n2)cc(OC)c1]
Compound-A76 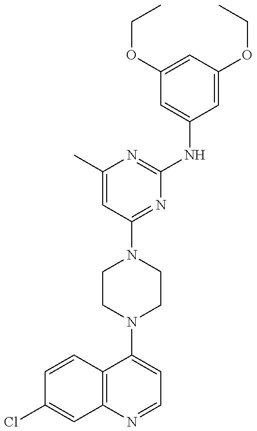 [SMILES: CCOc1cc(Nc2nc(C)cc(N3CCN(c4ccnc5cc(Cl)ccc45)CC3)n2)cc(OCC)c1]
Compound-A77 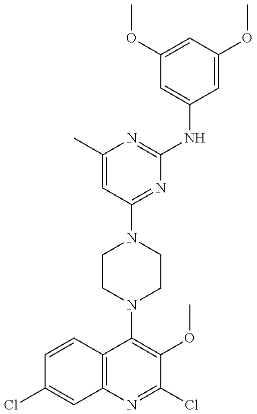 [SMILES: COc1cc(Nc2nc(C)cc(N3CCN(c4c(OC)c(Cl)nc5cc(Cl)ccc45)CC3)n2)cc(OC)c1]
Compound-A78 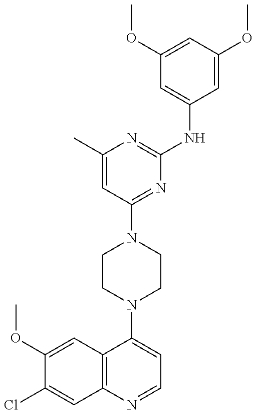 [SMILES: COc1cc(Nc2nc(C)cc(N3CCN(c4ccnc5cc(Cl)c(OC)cc45)CC3)n2)cc(OC)c1]
Compound-A79 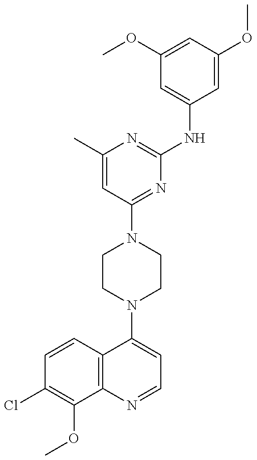 [SMILES: COc1cc(Nc2nc(C)cc(N3CCN(c4ccnc5c(OC)c(Cl)ccc45)CC3)n2)cc(OC)c1]
Compound-A80 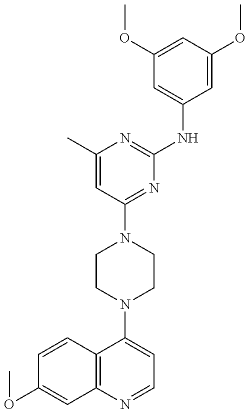 [SMILES: COc1cc(Nc2nc(C)cc(N3CCN(c4ccnc5cc(OC)ccc45)CC3)n2)cc(OC)c1]
Compound-A81 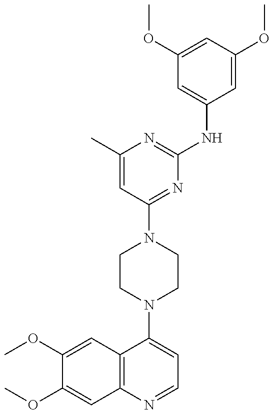
Compound-A82 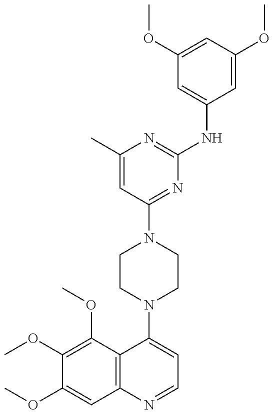
Compound-A83 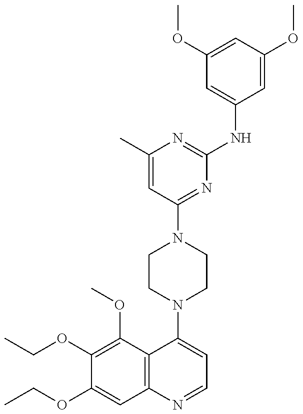 [SMILES: CCOc1cc2nccc(N3CCN(c4cc(C)nc(Nc5cc(OC)cc(OC)c5)n4)CC3)c2c(OC)c1OCC]
Compound-A84 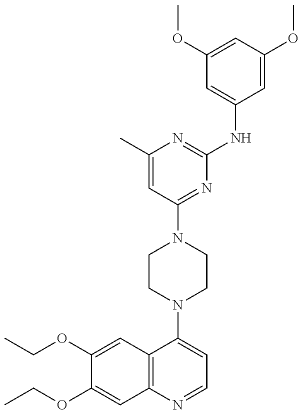 [SMILES: CCOc1cc2nccc(N3CCN(c4cc(C)nc(Nc5cc(OC)cc(OC)c5)n4)CC3)c2cc1OCC]
Compound-A85 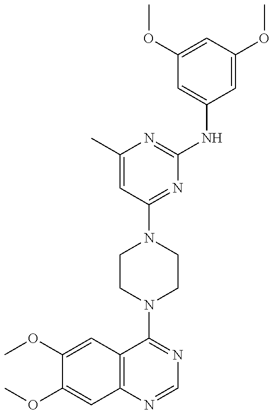
Compound-A86 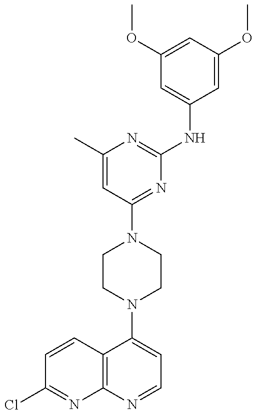 [SMILES: COc1cc(Nc2nc(C)cc(N3CCN(c4ccnc5nc(Cl)ccc45)CC3)n2)cc(OC)c1]
Compound-A87 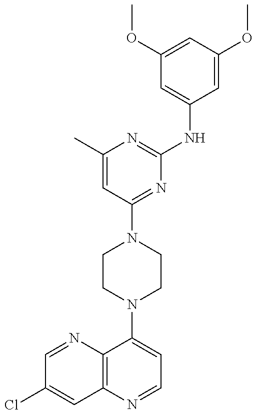 [SMILES: COc1cc(Nc2nc(C)cc(N3CCN(c4ccnc5cc(Cl)cnc45)CC3)n2)cc(OC)c1]
Compound-A88 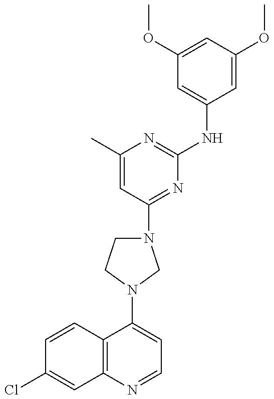 [SMILES: COc1cc(Nc2nc(C)cc(N3CCN(c4ccnc5cc(Cl)ccc45)C3)n2)cc(OC)c1]
Compound-A89 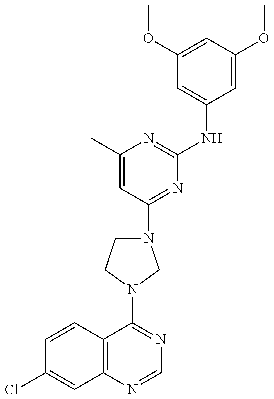 [SMILES: COc1cc(Nc2nc(C)cc(N3CCN(c4ncnc5cc(Cl)ccc45)C3)n2)cc(OC)c1]
Compound-A90 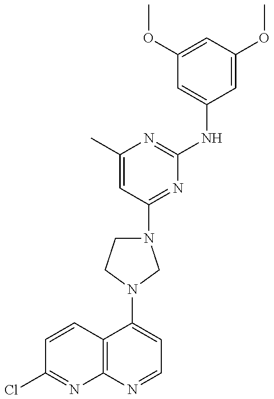 [SMILES: COc1cc(Nc2nc(C)cc(N3CCN(c4ccnc5nc(Cl)ccc45)C3)n2)cc(OC)c1]
Compound-A91 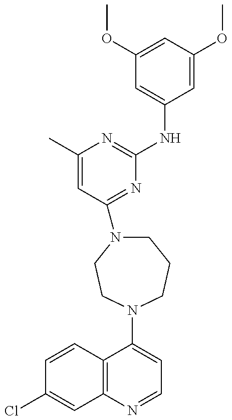 [SMILES: COc1cc(Nc2nc(C)cc(N3CCCN(c4ccnc5cc(Cl)ccc45)CC3)n2)cc(OC)c1]
Compound-A92 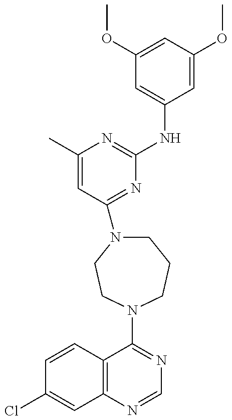 [SMILES: COc1cc(Nc2nc(C)cc(N3CCCN(c4ncnc5cc(Cl)ccc45)CC3)n2)cc(OC)c1]
Compound-A93 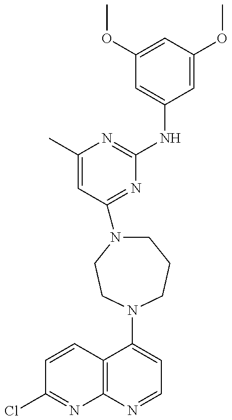 [SMILES: COc1cc(Nc2nc(C)cc(N3CCCN(c4ccnc5nc(Cl)ccc45)CC3)n2)cc(OC)c1]
Compound-A94 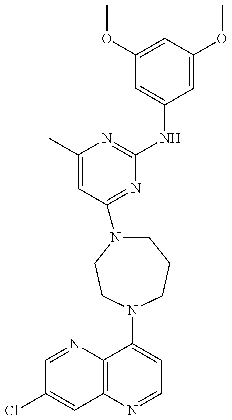 [SMILES: COc1cc(Nc2nc(C)cc(N3CCCN(c4ccnc5cc(Cl)cnc45)CC3)n2)cc(OC)c1]
Compound-A95 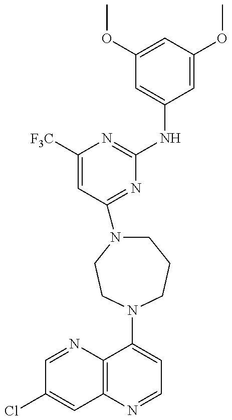 [SMILES: COc1cc(Nc2nc(N3CCCN(c4ccnc5cc(Cl)cnc45)CC3)cc(C(F)(F)F)n2)cc(OC)c1]
Compound-A96 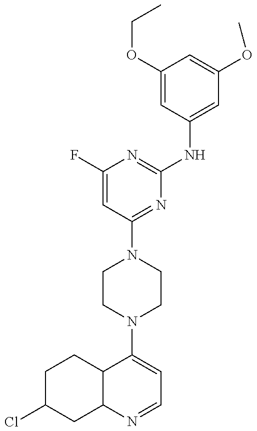 [SMILES: CCOc1cc(Nc2nc(F)cc(N3CCN(C4=CC=NC5CC(Cl)CCC45)CC3)n2)cc(OC)c1]
Compound-A97 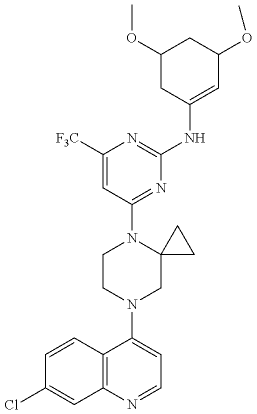 [SMILES: COC1C=C(Nc2nc(N3CCN(c4ccnc5cc(Cl)ccc45)CC34CC4)cc(C(F)(F)F)n2)CC(OC)C1]
Compound-A98 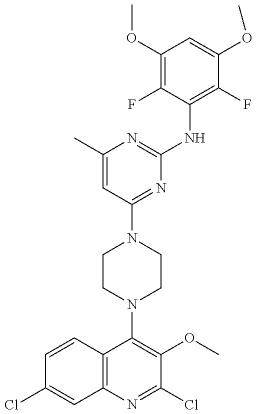 [SMILES: COc1cc(OC)c(F)c(Nc2nc(C)cc(N3CCN(c4c(OC)c(Cl)nc5cc(Cl)ccc45)CC3)n2)c1F]
Compound-A99 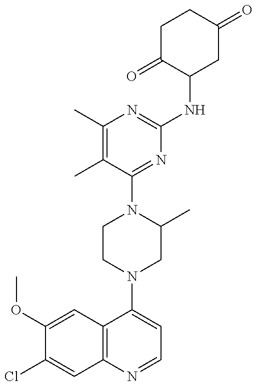 [SMILES: COc1cc2c(N3CCN(c4nc(NC5CC(=O)CCC5=O)nc(C)c4C)C(C)C3)ccnc2cc1Cl]
Compound-A100 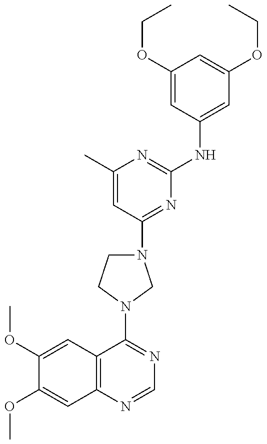 [SMILES: CCOc1cc(Nc2nc(C)cc(N3CCN(c4ncnc5cc(OC)c(OC)cc45)C3)n2)cc(OCC)c1]
Compound-A100 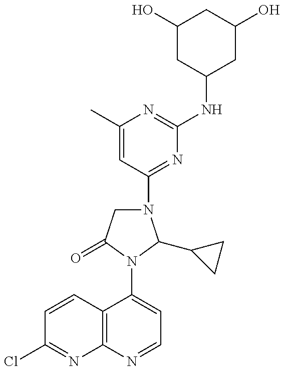 [SMILES: Cc1cc(N2CC(=O)N(c3ccnc4nc(Cl)ccc34)C2C2CC2)nc(NC2CC(O)CC(O)C2)n1]
Compound-A102 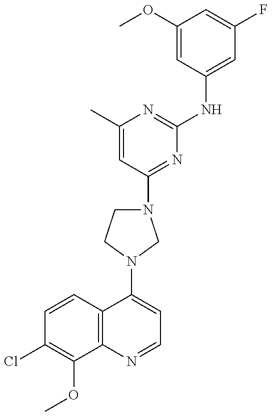 [SMILES: COc1cc(F)cc(Nc2nc(C)cc(N3CCN(c4ccnc5c(OC)c(Cl)ccc45)C3)n2)c1]
Compound-A103 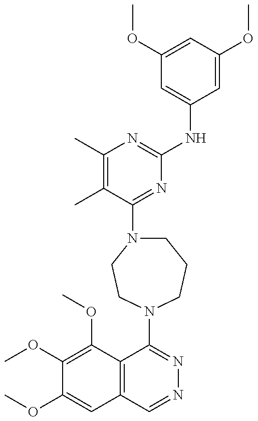
Compound-A104 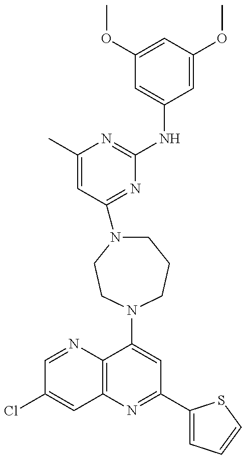 [SMILES: COc1cc(Nc2nc(C)cc(N3CCCN(c4cc(-c5cccs5)nc5cc(Cl)cnc45)CC3)n2)cc(OC)c1]
Compound-A105 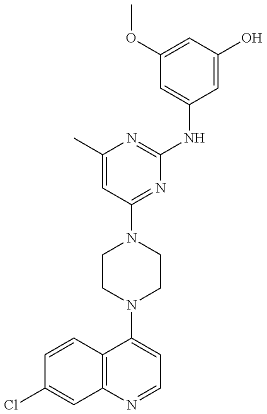 [SMILES: COc1cc(O)cc(Nc2nc(C)cc(N3CCN(c4ccnc5cc(Cl)ccc45)CC3)n2)c1]
Compound-A106 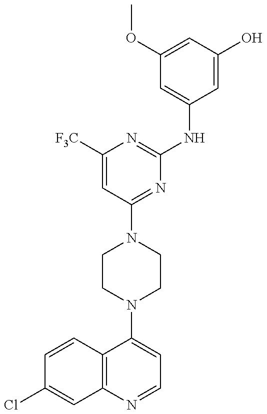 [SMILES: COc1cc(O)cc(Nc2nc(N3CCN(c4ccnc5cc(Cl)ccc45)CC3)cc(C(F)(F)F)n2)c1]
Compound-A107 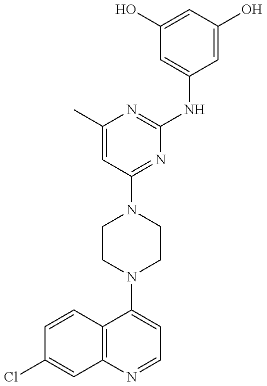 [SMILES: Cc1cc(N2CCN(c3ccnc4cc(Cl)ccc34)CC2)nc(Nc2cc(O)cc(O)c2)n1]
Compound-A108 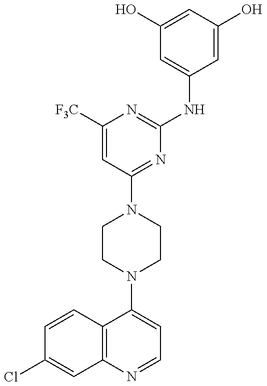 [SMILES: Oc1cc(O)cc(Nc2nc(N3CCN(c4ccnc5cc(Cl)ccc45)CC3)cc(C(F)(F)F)n2)c1]
Compound-A109 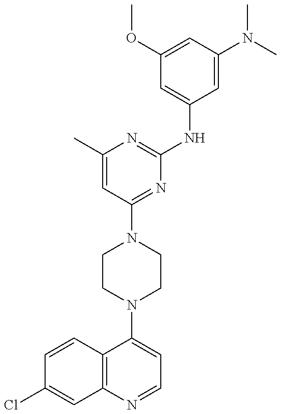 [SMILES: COc1cc(Nc2nc(C)cc(N3CCN(c4ccnc5cc(Cl)ccc45)CC3)n2)cc(N(C)C)c1]
Compound-A110 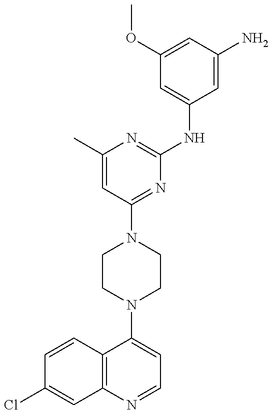 [SMILES: COc1cc(N)cc(Nc2nc(C)cc(N3CCN(c4ccnc5cc(Cl)ccc45)CC3)n2)c1]
Compound-A111 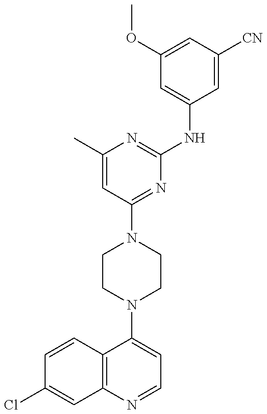 [SMILES: COc1cc(C#N)cc(Nc2nc(C)cc(N3CCN(c4ccnc5cc(Cl)ccc45)CC3)n2)c1]
Compound-A112 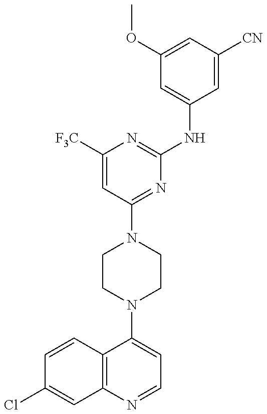 [SMILES: COc1cc(C#N)cc(Nc2nc(N3CCN(c4ccnc5cc(Cl)ccc45)CC3)cc(C(F)(F)F)n2)c1]
Compound-A113 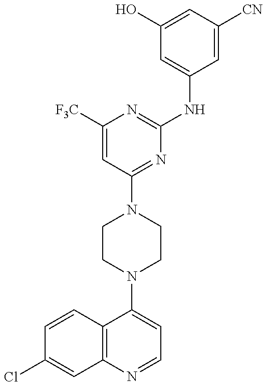 [SMILES: N#Cc1cc(O)cc(Nc2nc(N3CCN(c4ccnc5cc(Cl)ccc45)CC3)cc(C(F)(F)F)n2)c1]
Compound-A114 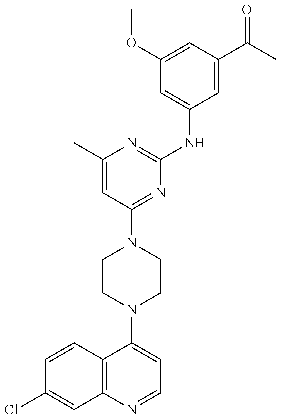 [SMILES: COc1cc(Nc2nc(C)cc(N3CCN(c4ccnc5cc(Cl)ccc45)CC3)n2)cc(C(C)=O)c1]
Compound-A115 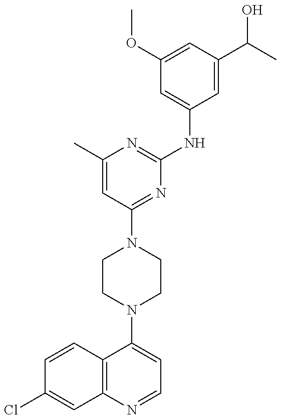 [SMILES: COc1cc(Nc2nc(C)cc(N3CCN(c4ccnc5cc(Cl)ccc45)CC3)n2)cc(C(C)O)c1]
Compound-A116

Preferably, in one of the embodiments the present invention provides a compound of Formula (A), wherein:
R1 is

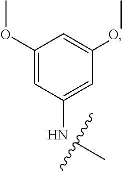

R2 is

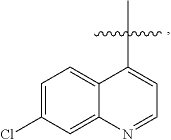

R3 is CF₃, R4 is H, R5 is H, R6 is H, R7 is H, R8 is H,
m=0 and n=0,
having chemical structure as represented by Compound A1,

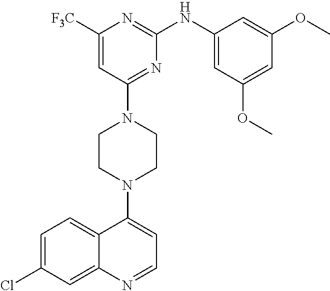

### Compound A1 {4-(4-(7-chloroquinolin-4-yl)piperazin-1-yl)-N-(3,5-dimethoxyphenyl)-6-(trifluoromethyl)pyridin-2-amine}

In another embodiment, the present invention provides a compound of Formula (A), wherein:
R1 is

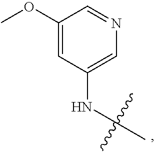

R2 is

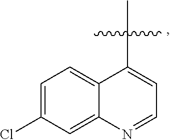

R3 is CF₃, R4 is H, R5 is H, R6 is H, R7 is H, R8 is H, m=0 and n=0,
having chemical structure as represented by Compound A7,

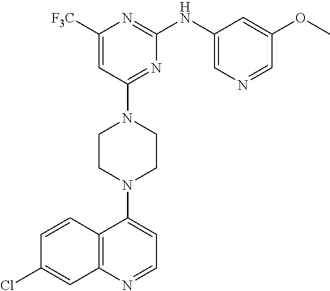

### Compound A7 {4-(4-(7-chloroquinolin-4-yl)piperazin-1-yl)-N-(5-methoxypyridin-3-yl)-6-(trifluoromethyl)pyrimidin-2-amine}

In another embodiment, the present invention provides a compound of Formula (A), wherein:
R1 is

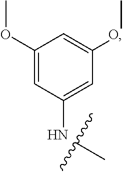

R2 is

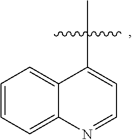

R3 is CH₃, R4 is H, R5 is H, R6 is H, R7 is H, R8 is H, m=0 and n=0,
having chemical structure as represented by Compound A42,

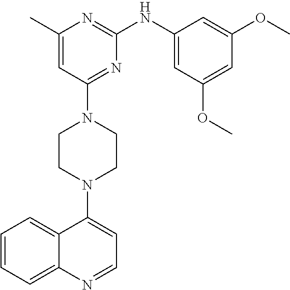

### Compound A42 {N-(3,5-dimethoxyphenyl)-4-methyl-6-(4-(quinolin-4-yl)piperazin-1-yl)pyrimidin-2-amine}

In another embodiment, the present invention provides a compound of Formula (A), wherein:
R1 is

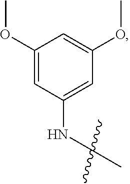 [SMILES: *Nc1cc(OC)cc(OC)c1]

R2 is

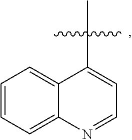

R3 is CF₃, R4 is H, R5 is H, R6 is H, R7 is H, R8 is H, m=0 and n=0,
having chemical structure as represented by Compound A43,

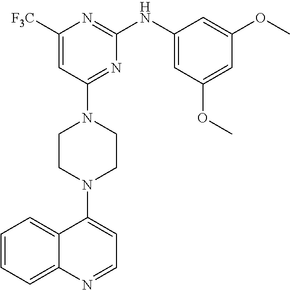

### Compound A43 {N-(3,5-dimethoxyphenyl)-4-(4-(quinolin-4-yl)piperazin-1-yl)-6-(trifluoromethyl)pyrimidin-2-amine}

In another embodiment, the present invention provides a compound of Formula (A), wherein:
R1 is

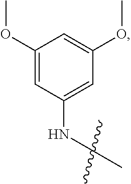 [SMILES: *Nc1cc(OC)cc(OC)c1]

R2 is

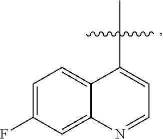 [SMILES: *c1ccnc2cc(F)ccc12]

R3 is CH₃, R4 is H, R5 is H, R6 is H, R7 is H, R8 is H, m=0 and n=0, having chemical structure as represented by Compound A48,

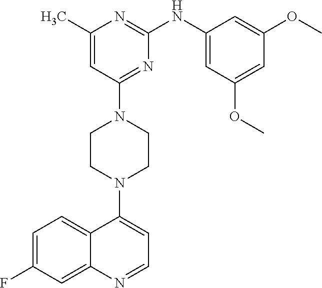 [SMILES: COc1cc(Nc2nc(C)cc(N3CCN(c4ccnc5cc(F)ccc45)CC3)n2)cc(OC)c1]

### Compound A48 {N-(3,5-dimethoxyphenyl)-4-(4-(7-fluoroquinolin-4-yl)piperazin-1-yl)-6-methylpyrimidin-2-amine}

In another embodiment, the present invention provides a compound of Formula (A), wherein:
R1 is

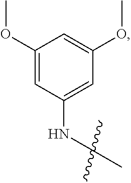 [SMILES: *Nc1cc(OC)cc(OC)c1]

R2 is

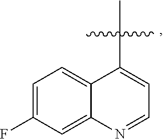 [SMILES: *c1ccnc2cc(F)ccc12]

R3 is CF₃, R4 is H, R5 is H, R6 is H, R7 is H, R8 is H, m=0 and n=0, having chemical structure as represented by Compound A49,

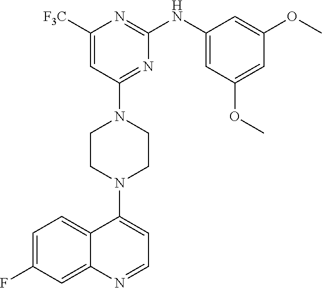 [SMILES: COc1cc(Nc2nc(N3CCN(c4ccnc5cc(F)ccc45)CC3)cc(C(F)(F)F)n2)cc(OC)c1]

### Compound A49{N-(3,5-dimethoxyphenyl)-4-(4-(7-fluoroquinolin-4-yl)piperazin-1-yl)-6-(trifluoromethyl)pyrimidin-2-amine}

### EXAMPLES

### Example 1

Quinoline Piperazine Derivatives, Method of Preparation and their Properties

### General Procedure for the Synthesis of Intermediates 3a-3ap

A well dried round bottom flask was charged with 1a-1ab (Table 3) and dissolved in ethanol. To this 2a-2g (Table 3) were added portion wise and allowed to stir at 70° C. for 12 h. The progress of the reaction was monitored by TLC. After completion of the reaction, the ethanol was evaporated under vacuum, then the reaction mixture was washed with NaHCO₃ solution and extracted with DCM (3×20 mL). The extracted three fractions were collected, dried over Na₂SO₄ and concentrated to get the desired compounds.

TABLE 3
List of materials and intermediates

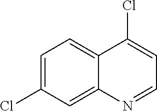 [SMILES: Clc1ccc2c(Cl)ccnc2c1]

1a

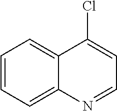 [SMILES: Clc1ccnc2ccccc12]

1b

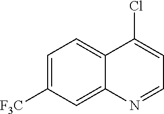 [SMILES: FC(F)(F)c1ccc2c(Cl)ccnc2c1]

1c

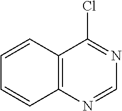 [SMILES: Clc1ncnc2ccccc12]

1d

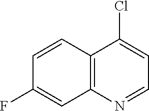 [SMILES: Fc1ccc2c(Cl)ccnc2c1]

1e

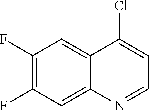 [SMILES: Fc1cc2nccc(Cl)c2cc1F]

1f

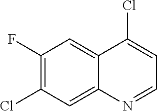 [SMILES: Fc1cc2c(Cl)ccnc2cc1Cl]

1g

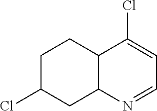 [SMILES: ClC1=CC=NC2CC(Cl)CCC12]

1h

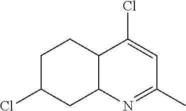

1i

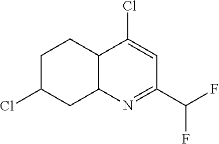 [SMILES: FC(F)C1=NC2CC(Cl)CCC2C(Cl)=C1]

1j

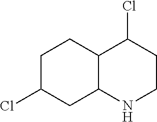 [SMILES: ClC1CCC2C(Cl)CCNC2C1]

1k

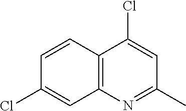

1l

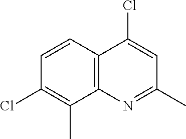

1m

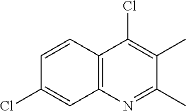

1n

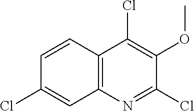

1o

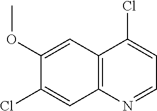

1p

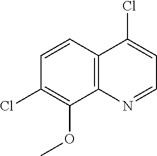

1q

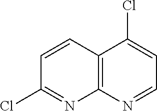 [SMILES: Clc1ccc2c(Cl)ccnc2n1]

1x

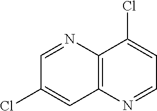 [SMILES: Clc1cnc2c(Cl)ccnc2c1]

1y

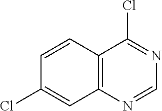 [SMILES: Clc1ccc2c(Cl)ncnc2c1]

1z

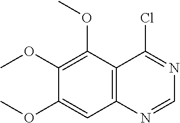 [SMILES: COc1cc2ncnc(Cl)c2c(OC)c1OC]

1aa

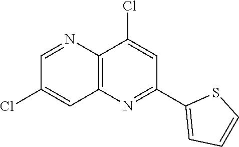 [SMILES: Clc1cnc2c(Cl)cc(-c3cccs3)nc2c1]

1ab

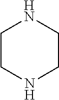

2a

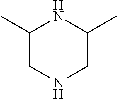

2b

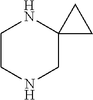

2c

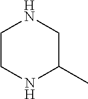

2d

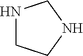

2e

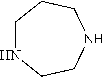

2f

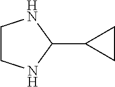

2g

TABLE 4
List of intermediates 3a-3ap

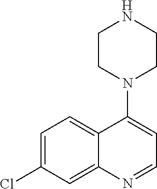 [SMILES: Clc1ccc2c(N3CCNCC3)ccnc2c1]

3a

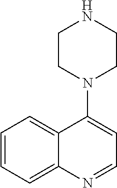 [SMILES: c1ccc2c(N3CCNCC3)ccnc2c1]

3b

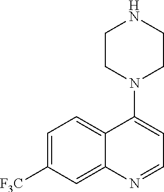 [SMILES: FC(F)(F)c1ccc2c(N3CCNCC3)ccnc2c1]

3c

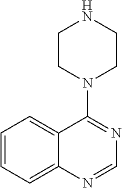 [SMILES: c1ccc2c(N3CCNCC3)ncnc2c1]

3d

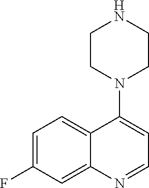 [SMILES: Fc1ccc2c(N3CCNCC3)ccnc2c1]

3e

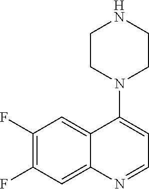 [SMILES: Fc1cc2nccc(N3CCNCC3)c2cc1F]

3f

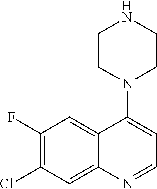 [SMILES: Fc1cc2c(N3CCNCC3)ccnc2cc1Cl]

3g

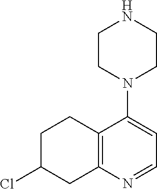 [SMILES: ClC1CCc2c(N3CCNCC3)ccnc2C1]

3h

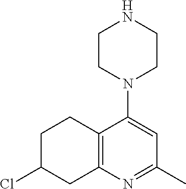 [SMILES: Cc1cc(N2CCNCC2)c2c(n1)CC(Cl)CC2]

3i

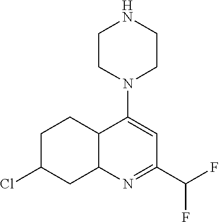 [SMILES: FC(F)C1=NC2CC(Cl)CCC2C(N2CCNCC2)=C1]

3j

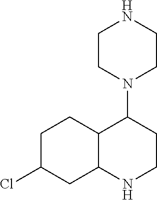 [SMILES: ClC1CCC2C(C1)NCCC2N1CCNCC1]

3k

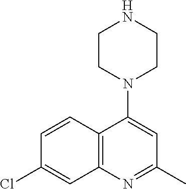 [SMILES: Cc1cc(N2CCNCC2)c2ccc(Cl)cc2n1]

3l

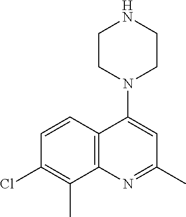 [SMILES: Cc1cc(N2CCNCC2)c2ccc(Cl)c(C)c2n1]

3m

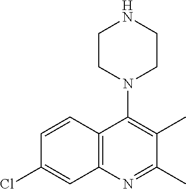 [SMILES: Cc1nc2cc(Cl)ccc2c(N2CCNCC2)c1C]

3n

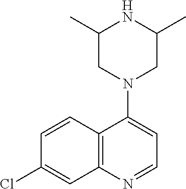 [SMILES: CC1CN(c2ccnc3cc(Cl)ccc23)CC(C)N1]

3o

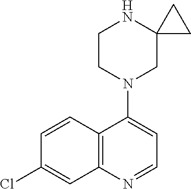 [SMILES: Clc1ccc2c(N3CCNC4(CC4)C3)ccnc2c1]

3p

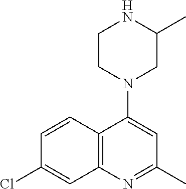 [SMILES: Cc1cc(N2CCNC(C)C2)c2ccc(Cl)cc2n1]

3q

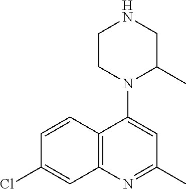 [SMILES: Cc1cc(N2CCNCC2C)c2ccc(Cl)cc2n1]

3r

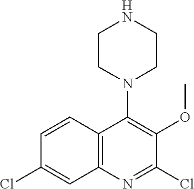 [SMILES: COc1c(Cl)nc2cc(Cl)ccc2c1N1CCNCC1]

3s

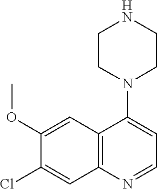 [SMILES: COc1cc2c(N3CCNCC3)ccnc2cc1Cl]

3t

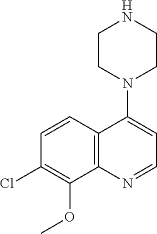 [SMILES: COc1c(Cl)ccc2c(N3CCNCC3)ccnc12]

3u

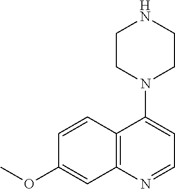 [SMILES: COc1ccc2c(N3CCNCC3)ccnc2c1]

3v

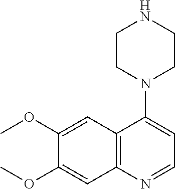 [SMILES: COc1cc2nccc(N3CCNCC3)c2cc1OC]

3w

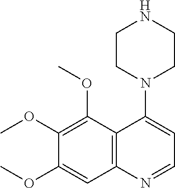 [SMILES: COc1cc2nccc(N3CCNCC3)c2c(OC)c1OC]

3x

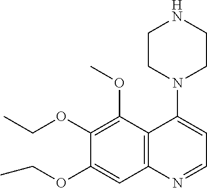 [SMILES: CCOc1cc2nccc(N3CCNCC3)c2c(OC)c1OCC]

3y

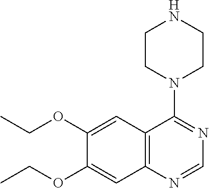 [SMILES: CCOc1cc2ncnc(N3CCNCC3)c2cc1OCC]

3z

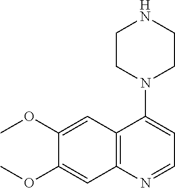 [SMILES: COc1cc2nccc(N3CCNCC3)c2cc1OC]

3aa

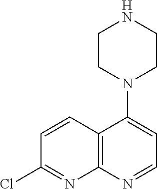 [SMILES: Clc1ccc2c(N3CCNCC3)ccnc2n1]

3ab

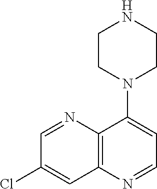 [SMILES: Clc1cnc2c(N3CCNCC3)ccnc2c1]

3ac

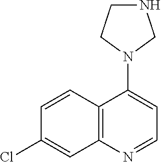 [SMILES: Clc1ccc2c(N3CCNC3)ccnc2c1]

3ad

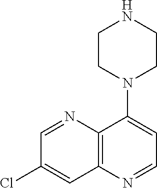 [SMILES: Clc1cnc2c(N3CCNCC3)ccnc2c1]

3ae

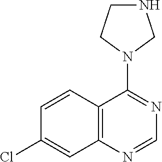 [SMILES: Clc1ccc2c(N3CCNC3)ncnc2c1]

3af

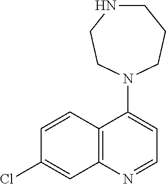 [SMILES: Clc1ccc2c(N3CCCNCC3)ccnc2c1]

3ag

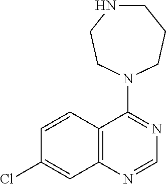 [SMILES: Clc1ccc2c(N3CCCNCC3)ncnc2c1]

3ah

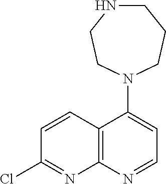 [SMILES: Clc1ccc2c(N3CCCNCC3)ccnc2n1]

3ai

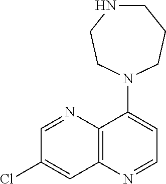 [SMILES: Clc1cnc2c(N3CCCNCC3)ccnc2c1]

3aj

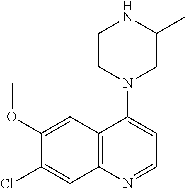 [SMILES: COc1cc2c(N3CCNC(C)C3)ccnc2cc1Cl]

3ak

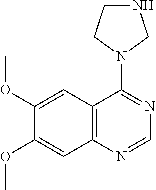 [SMILES: COc1cc2ncnc(N3CCNC3)c2cc1OC]

3al

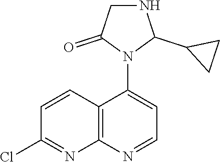 [SMILES: O=C1CNC(C2CC2)N1c1ccnc2nc(Cl)ccc12]

3am

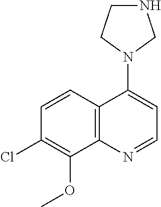 [SMILES: COc1c(Cl)ccc2c(N3CCNC3)ccnc12]

3an

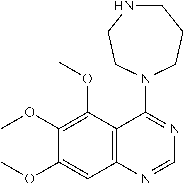 [SMILES: COc1cc2ncnc(N3CCCNCC3)c2c(OC)c1OC]

3ao

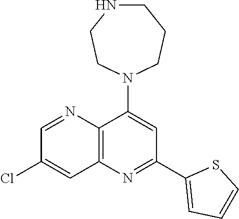 [SMILES: Clc1cnc2c(N3CCCNCC3)cc(-c3cccs3)nc2c1]

3ap

### General Procedure for the Synthesis of Intermediates 5a-5be

Intermediates 3a-3ap were charged in a well dried round bottom flask and dissolved in ethanol followed by the addition of 4a-4h at 0° C. Then the reaction mixture was allowed to stir for 4 h at room temperature. The progress of the reaction was monitored by TLC. After completion of the reaction ethanol was evaporated under reduced pressure, the residue was washed with NaHCO₃ solution and extracted with DCM (3×20 mL). The combined organic fractions were collected, dried over Na₂SO₄ and concentrated on rotary evaporator. The crude compound was purified by column chromatography using EtOAc:Hexane (3:7) as eluents to afford the desired compounds.

TABLE 5
List of intermediates 5a-5be

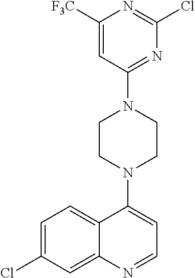 [SMILES: FC(F)(F)c1cc(N2CCN(c3ccnc4cc(Cl)ccc34)CC2)nc(Cl)n1]

5a

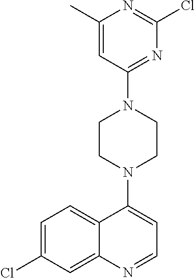 [SMILES: Cc1cc(N2CCN(c3ccnc4cc(Cl)ccc34)CC2)nc(Cl)n1]

5b

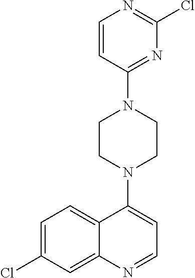 [SMILES: Clc1ccc2c(N3CCN(c4ccnc(Cl)n4)CC3)ccnc2c1]

5c

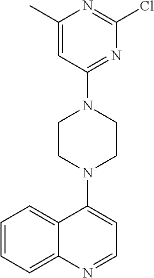 [SMILES: Cc1cc(N2CCN(c3ccnc4ccccc34)CC2)nc(Cl)n1]

5d

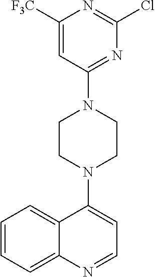 [SMILES: FC(F)(F)c1cc(N2CCN(c3ccnc4ccccc34)CC2)nc(Cl)n1]

5e

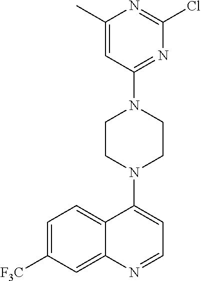 [SMILES: Cc1cc(N2CCN(c3ccnc4cc(C(F)(F)F)ccc34)CC2)nc(Cl)n1]

5f

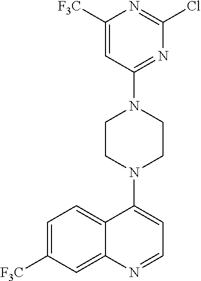 [SMILES: FC(F)(F)c1ccc2c(N3CCN(c4cc(C(F)(F)F)nc(Cl)n4)CC3)ccnc2c1]

5g

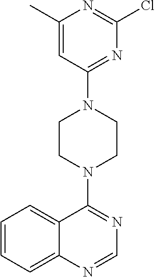 [SMILES: Cc1cc(N2CCN(c3ncnc4ccccc34)CC2)nc(Cl)n1]

5h

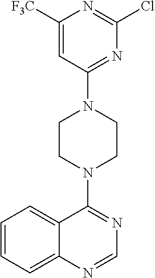 [SMILES: FC(F)(F)c1cc(N2CCN(c3ncnc4ccccc34)CC2)nc(Cl)n1]

5i

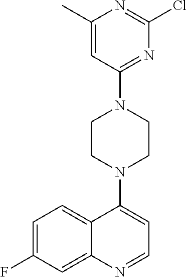 [SMILES: Cc1cc(N2CCN(c3ccnc4cc(F)ccc34)CC2)nc(Cl)n1]

5j

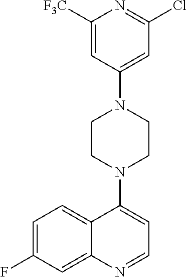 [SMILES: Fc1ccc2c(N3CCN(c4cc(Cl)nc(C(F)(F)F)c4)CC3)ccnc2c1]

5k

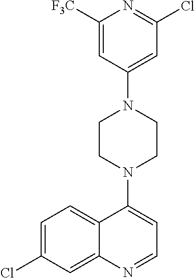 [SMILES: FC(F)(F)c1cc(N2CCN(c3ccnc4cc(Cl)ccc34)CC2)cc(Cl)n1]

5l

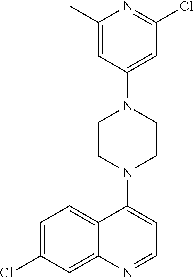 [SMILES: Cc1cc(N2CCN(c3ccnc4cc(Cl)ccc34)CC2)cc(Cl)n1]

5m

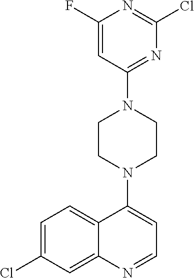 [SMILES: Fc1cc(N2CCN(c3ccnc4cc(Cl)ccc34)CC2)nc(Cl)n1]

5n

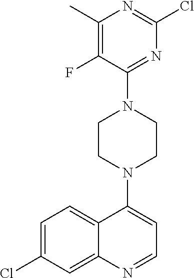 [SMILES: Cc1nc(Cl)nc(N2CCN(c3ccnc4cc(Cl)ccc34)CC2)c1F]

5o

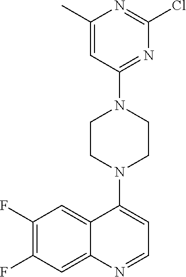 [SMILES: Cc1cc(N2CCN(c3ccnc4cc(F)c(F)cc34)CC2)nc(Cl)n1]

5p

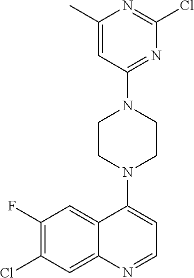 [SMILES: Cc1cc(N2CCN(c3ccnc4cc(Cl)c(F)cc34)CC2)nc(Cl)n1]

5q

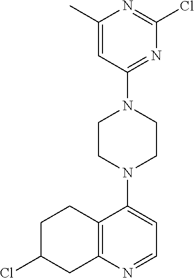 [SMILES: Cc1cc(N2CCN(c3ccnc4c3CCC(Cl)C4)CC2)nc(Cl)n1]

5r

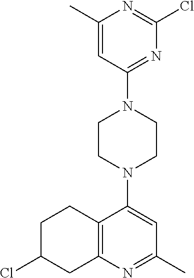 [SMILES: Cc1cc(N2CCN(c3cc(C)nc4c3CCC(Cl)C4)CC2)nc(Cl)n1]

5s

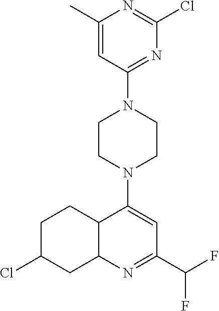 [SMILES: Cc1cc(N2CCN(C3=CC(C(F)F)=NC4CC(Cl)CCC34)CC2)nc(Cl)n1]

5t

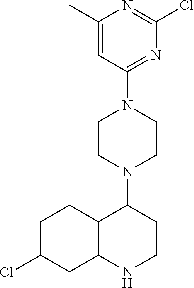 [SMILES: Cc1cc(N2CCN(C3CCNC4CC(Cl)CCC43)CC2)nc(Cl)n1]

5u

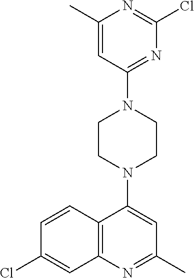 [SMILES: Cc1cc(N2CCN(c3cc(C)nc4cc(Cl)ccc34)CC2)nc(Cl)n1]

5v

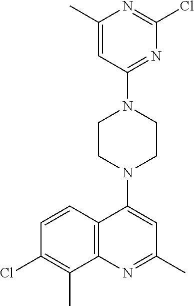 [SMILES: Cc1cc(N2CCN(c3cc(C)nc4c(C)c(Cl)ccc34)CC2)nc(Cl)n1]

5w

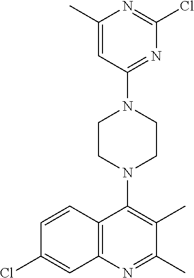 [SMILES: Cc1cc(N2CCN(c3c(C)c(C)nc4cc(Cl)ccc34)CC2)nc(Cl)n1]

5x

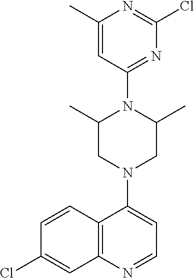 [SMILES: Cc1cc(N2C(C)CN(c3ccnc4cc(Cl)ccc34)CC2C)nc(Cl)n1]

5y

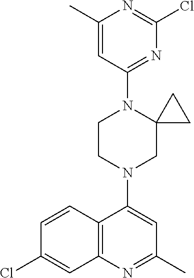 [SMILES: Cc1cc(N2CCN(c3cc(C)nc4cc(Cl)ccc34)CC23CC3)nc(Cl)n1]

5z

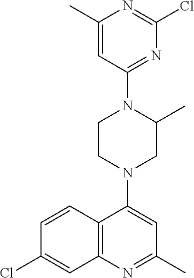 [SMILES: Cc1cc(N2CCN(c3cc(C)nc4cc(Cl)ccc34)CC2C)nc(Cl)n1]

5aa

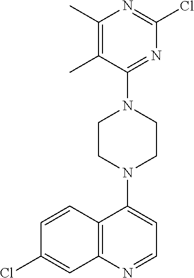 [SMILES: Cc1nc(Cl)nc(N2CCN(c3ccnc4cc(Cl)ccc34)CC2)c1C]

5ab

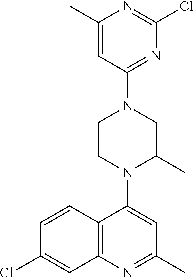 [SMILES: Cc1cc(N2CCN(c3cc(C)nc4cc(Cl)ccc34)C(C)C2)nc(Cl)n1]

5ac

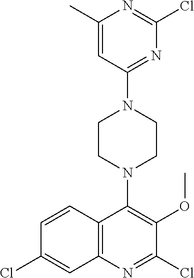 [SMILES: COc1c(Cl)nc2cc(Cl)ccc2c1N1CCN(c2cc(C)nc(Cl)n2)CC1]

5ad

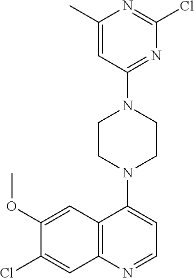 [SMILES: COc1cc2c(N3CCN(c4cc(C)nc(Cl)n4)CC3)ccnc2cc1Cl]

5ae

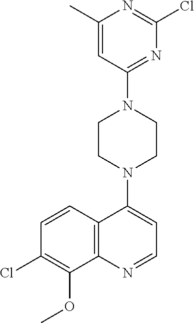 [SMILES: COc1c(Cl)ccc2c(N3CCN(c4cc(C)nc(Cl)n4)CC3)ccnc12]

5af

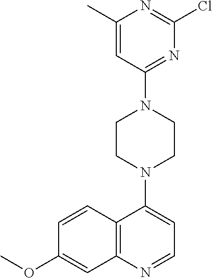 [SMILES: COc1ccc2c(N3CCN(c4cc(C)nc(Cl)n4)CC3)ccnc2c1]

5ag

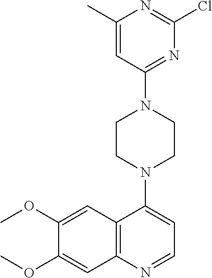 [SMILES: COc1cc2nccc(N3CCN(c4cc(C)nc(Cl)n4)CC3)c2cc1OC]

5ah

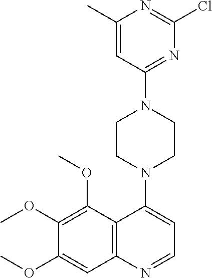 [SMILES: COc1cc2nccc(N3CCN(c4cc(C)nc(Cl)n4)CC3)c2c(OC)c1OC]

5ai

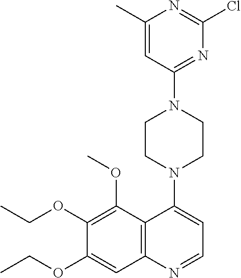 [SMILES: CCOc1cc2nccc(N3CCN(c4cc(C)nc(Cl)n4)CC3)c2c(OC)c1OCC]

5aj

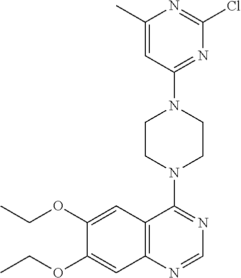 [SMILES: CCOc1cc2ncnc(N3CCN(c4cc(C)nc(Cl)n4)CC3)c2cc1OCC]

5ak

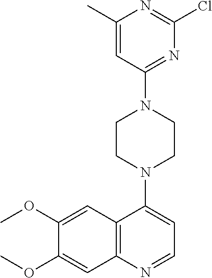 [SMILES: COc1cc2nccc(N3CCN(c4cc(C)nc(Cl)n4)CC3)c2cc1OC]

5al

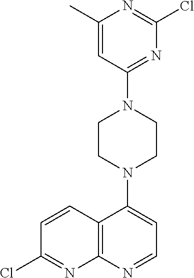 [SMILES: Cc1cc(N2CCN(c3ccnc4nc(Cl)ccc34)CC2)nc(Cl)n1]

5am

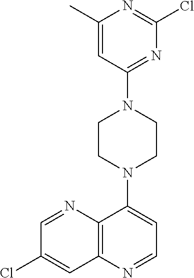 [SMILES: Cc1cc(N2CCN(c3ccnc4cc(Cl)cnc34)CC2)nc(Cl)n1]

5an

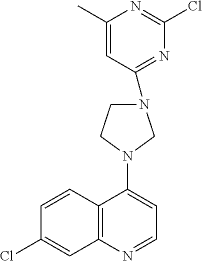 [SMILES: Cc1cc(N2CCN(c3ccnc4cc(Cl)ccc34)C2)nc(Cl)n1]

5ao

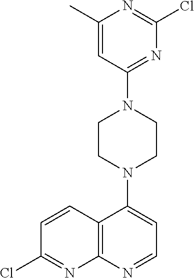 [SMILES: Cc1cc(N2CCN(c3ccnc4nc(Cl)ccc34)CC2)nc(Cl)n1]

5ap

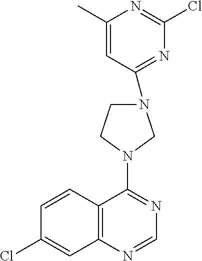 [SMILES: Cc1cc(N2CCN(c3ncnc4cc(Cl)ccc34)C2)nc(Cl)n1]

5aq

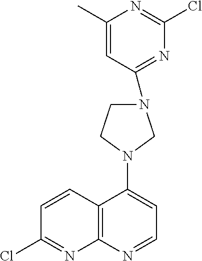 [SMILES: Cc1cc(N2CCN(c3ccnc4nc(Cl)ccc34)C2)nc(Cl)n1]

5ar

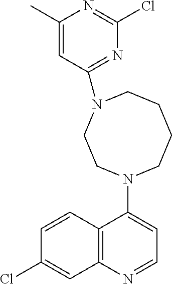 [SMILES: Cc1cc(N2CCCCN(c3ccnc4cc(Cl)ccc34)CC2)nc(Cl)n1]

5as

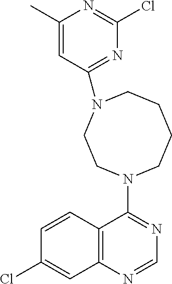 [SMILES: Cc1cc(N2CCCCN(c3ncnc4cc(Cl)ccc34)CC2)nc(Cl)n1]

5at

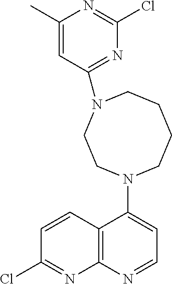 [SMILES: Cc1cc(N2CCCCN(c3ccnc4nc(Cl)ccc34)CC2)nc(Cl)n1]

5au

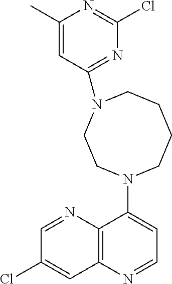 [SMILES: Cc1cc(N2CCCCN(c3ccnc4cc(Cl)cnc34)CC2)nc(Cl)n1]

5av

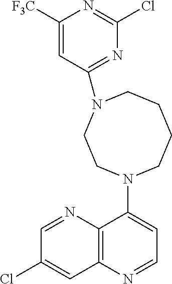 [SMILES: FC(F)(F)c1cc(N2CCCCN(c3ccnc4cc(Cl)cnc34)CC2)nc(Cl)n1]

5aw

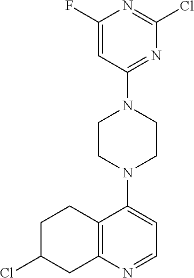 [SMILES: Fc1cc(N2CCN(c3ccnc4c3CCC(Cl)C4)CC2)nc(Cl)n1]

5ax

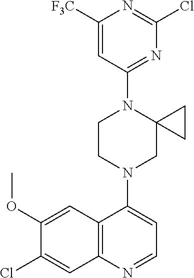 [SMILES: COc1cc2c(N3CCN(c4cc(C(F)(F)F)nc(Cl)n4)C4(CC4)C3)ccnc2cc1Cl]

5ay

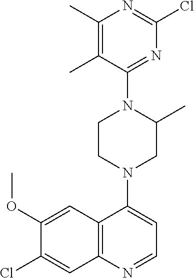 [SMILES: COc1cc2c(N3CCN(c4nc(Cl)nc(C)c4C)C(C)C3)ccnc2cc1Cl]

5az

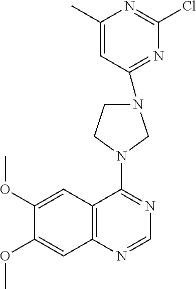 [SMILES: COc1cc2ncnc(N3CCN(c4cc(C)nc(Cl)n4)C3)c2cc1OC]

5ba

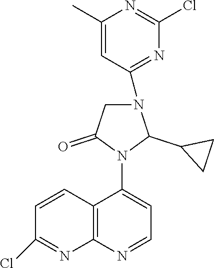 [SMILES: Cc1cc(N2CC(=O)N(c3ccnc4nc(Cl)ccc34)C2C2CC2)nc(Cl)n1]

5bb

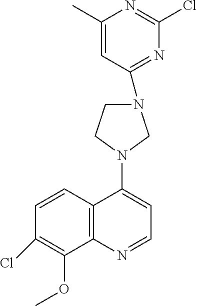 [SMILES: COc1c(Cl)ccc2c(N3CCN(c4cc(C)nc(Cl)n4)C3)ccnc12]

5bc

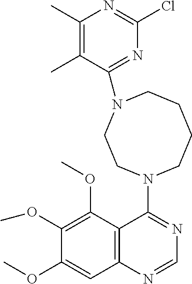 [SMILES: COc1cc2ncnc(N3CCCCN(c4nc(Cl)nc(C)c4C)CC3)c2c(OC)c1OC]

5bd

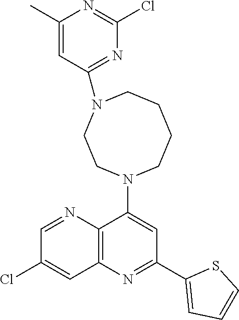 [SMILES: Cc1cc(N2CCCCN(c3cc(-c4cccs4)nc4cc(Cl)cnc34)CC2)nc(Cl)n1]

5be

### General Procedure for the Synthesis of A2, A3, A4, A5, A10, A27, A28, A29, A30, A31, A32, A39, A41, A42, A44, A46, A48, A51, A53, A54, A55, A56, A57, A58, A59, A60, A67, A68, A69, A70, A71, A72, A73, A74, A75, A76, A77, A78, A79, A80, A81, A82, A83, A84, A85, A86, A87, A88, A89, A90, A91, A92, A93, A94, A95, A99, A101, A102, A103, A104, A105, A106, A108, A110, A111, A112, A115, A116, A117

Intermediates 5b, 5c, 5d, 5f, 5 h, 5j, 5m, 5o, 5p, 5q, 5v, 5w, 5x, 5y, 5z, 5aa, 5ba, 5r, 5s, 5t, 5u, 5ac, 5az, 5ab, 5ad, 5ae, 5af, 5ag, 5ah, 5ai, 5aj, 5ak, 5al, 5am, 5an, 5ao, 5aw, 5be, 5ap, 5aq, 5au, 5ar, 5bb, 5bc, 5aq, 5bd, 5as, 5at, 5au (1 eq) were taken in a well dried round bottom flask and dissolved in Isopropanol followed by the addition of aromatic amines (1.1 eq). To this catalytic amount of Conc. HCl was added, the reaction mixture was allowed to reflux at 100° C. for 12 hours and the progress of the reaction was monitored by thin layer chromatography (TLC). After completion, reaction mixture was concentrated under vacuum. The residue was treated with NaHCO₃ solution and extracted in dichloromethane (DCM) (3×15 mL). The combined organic layer was washed with brine solution, dried over anhydrous Na₂SO₄ and concentrated under reduced pressure. Then the crude compound was purified by column chromatography to obtain the desired products.

### General Procedure for the Synthesis of A1, A6, A7, A8, A9, A33, A34, A35, A36, A37, A38, A40, A43, A45, A47, A49, A50, A52, A96, A107, A109, A113, A114

5a, 5e, 5g, 5i, 5k, 5l, 5n, 5av, 5ax, 5ay, 5aw (1 eq) were taken in a well dried round bottom flask and dissolved in 1,4-dioxane followed by the addition of aromatic amine (1.1 eq). To this KO^{t}Bu (1 eq) followed by catalytic amount of Pd(^{t}Bu₃P)₂ were added and the reaction mixture was allowed to reflux at 100° C. for 12 hours. The progress of the reaction was monitored by TLC. After completion, reaction mixture was concentrated under vacuum. Then the crude compound was purified by column chromatography to obtain the desired compound.

### General Procedure for the Synthesis of A19, A20, A21, A22, A23, A24, A25, A26, A97, A98

Intermediates 5a, 5aw, 5ax, (1 eq) were taken in a well dried round bottom flask and dissolved in N,N-Dimethylformamide followed by the addition of aliphatic amine (3 eq). Then the reaction mixture was allowed to reflux at 110° C. for 12 hours. The progress of the reaction was monitored by TLC. After completion, reaction mixture was cooled down to room temperature. To this 30-40 mL of water was added. Then the resulting residue was filtered and washed with excess cold water to obtain the desired compound.

### General Procedure for the Synthesis of A11, A12, A13, A14, A15, A16, A17, A18, A61, A62, A63, A64, A65, A66, A98, A100, A102

Intermediates 5b, 5ay, 5ba, (1 eq) were taken in a well dried round bottom flask and dissolved in in N,N-Dimethylformamide followed by the addition of aliphatic amine (3 eq). Then the reaction mixture was allowed to stir at 110° C. for 12 hours. The progress of the reaction was monitored by TLC. After completion, reaction mixture was cooled down to room temperature. To this 30-40 mL of water was added. The crude compound was filtered and washed with excess cold water and was purified by column chromatography to obtain the desired product.

### Analytical Data of the Compounds:

Compound-A1: 4-(4-(7-chloroquinolin-4-yl)piperazin-1-yl)-N-(3,5-dimethoxyphenyl)-6-(trifluoromethyl)pyridin-2-amine: ¹H-NMR-400-MHz-DMSO: 8.36 (d, 1H), 8.23 (d, 1H), 7.87 (s, 1H), 7.36 (d, 1H), 6.44 (d, 1H), 5.77 (d, 1H), 5.73 (s, 2H), 5.74 (d, 1H), 4.00 (s, 1H), 3.83 (s, 6H), 3.28 (s, 8H); ¹³C-NMR-400-MHz-DMSO: 160.6, 159.4, 159.2, 155.0, 153.0, 150.8, 144.4, 147.3, 135.6, 130.1, 128.6, 126.0, 122.6, 119.7, 118.3, 98.9, 92.9, 91.7, 90.4, 55.8, 49.8, 48.5; Molecular weight: 490.188

Compound-A2:4-(4-(7-chloroquinolin-4-yl)piperazin-1-yl)-N-(3-methoxy-5-(trifluoromethyl)phenyl)-6-methylpyrimidin-2-amine: ¹H-NMR-400-MHz-DMSO: 9.43 (d, 1H), 8.70 (d, 1H), 7.74 (d, 1H), 7.38 (d, 1H) 6.82 (d, 2H), 6.74 (d, 1H), 5.82 (s, 1H), 3.81 (s, 3H), 3.28 (s, 8H) 2.42 (s, 3H); ¹³C-NMR-400-MHz-DMSO: 170.2, 168.0, 164.5, 161.7, 159.2, 153.0, 150.8, 143.7, 135.6, 132.8, 130.1, 128.9, 126.0, 124.4, 122.6, 118.6, 108.3, 102.7, 99.2, 55.8, 49.8, 48.5, 23.9; Molecular weight: 528.165.

Compound-A3: 4-(4-(7-chloroquinolin-4-yl)piperazin-1-yl)-N-(5-methoxypyridin-3-yl)-6-methylpyrimidin-2-amine: ¹H-NMR-400-MHz-DMSO: 8.70 (d, 1H), 8.45 (d, 1H), 8.18 (s, 1H), 7.93 (s, 1H) 7.80 (s, 1H), 7.74 (d, 1H), 7.38 (d, 1H), 6.89 (d, 1H), 6.74 (s, 1H), 5.82 (s, 1H), 3.83 (s, 3H), 3.28 (s, 8H) 2.42 (s, 3H); ¹³C-NMR-400-MHz-DMSO: 170.2, 168.0, 164.5, 159.2, 156.1, 153.0, 150.8, 135.6, 134.2, 130.0, 126.9, 126.0, 122.6, 121.1, 118.3, 105.3, 93.8, 55.8, 49.8, 48.5, 23.9; Molecular weight: 461.173.

Compound-A4:4-(4-(7-chloroquinolin-4-yl)piperazin-1-yl)-N-(3-methoxyphenyl)-6-methylpyrimidin-2-amine: ¹H-NMR-400-MHz-DMSO: 9.43 (d, 1H), 8.70 (d, 1H), 8.45 (d, 1H), 7.74 (d, 1H), 7.38 (d, 1H) 7.21 (t, 1H), 6.82 (d, 1H), 6.74 (t, 1H), 6.49 (t, 1H), 5.82 (s, 1H), 3.74 (s, 3H), 3.28 (s, 8H) 2.42 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9; Molecular weight: 460.966.

Compound-A5:4-(4-(7-chloroquinolin-4-yl)piperazin-1-yl)-N-(3-fluoro-5-methoxyphenyl)-6-methylpyrimidin-2-amine: ¹H-NMR-400-MHz-DMSO: 9.43 (s, 1H), 8.70 (d, 1H), 8.45 (d, 1H), 7.74 (d, 1H), 7.38 (d, 1H) 7.21 (t, 1H), 6.79 (m, 2H), 6.59 (t, 1H), 5.82 (s, 1H), 3.81 (s, 3H), 3.28 (s, 8H) 2.42 (s, 3H); ¹³C-NMR-400-MHz-DMSO: 170.2, 168.0, 164.5, 163.0, 159.2, 153.0, 150.8, 145.0, 135.6, 130.1, 128.9, 126.0, 122.6, 118.3, 97.1, 95.0, 92.4, 55.8, 49.8, 48.5, 23.9; Molecular weight: 478.168.

Compound-A6:4-(4-(7-chloroquinolin-4-yl)piperazin-1-yl)-N-(3-methoxy-5-(trifluoromethyl)phenyl)-6-(trifluoromethyl)pyrimidin-2-amine: ¹H-NMR-400-MHz-DMSO: 9.43 (s, 1H), 8.70 (d, 1H), 8.45 (d, 1H), 7.74 (d, 1H), 7.38 (d, 1H) 7.21 (t, 1H), 6.79 (m, 2H), 6.59 (t, 1H), 5.82 (s, 1H), 3.81 (s, 3H), 3.28 (s, 8H) 2.42 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 582.137.

Compound-A7:4-(4-(7-chloroquinolin-4-yl)piperazin-1-yl)-N-(5-methoxypyridin-3-yl)-6-(trifluoromethyl)pyrimidin-2-amine: ¹H-NMR-400-MHz-DMSO: 9.43 (s, 1H), 8.70 (d, 1H), 8.45 (d, 1H), 7.74 (d, 1H), 7.38 (d, 1H), 7.21 (t, 1H), 6.79 (m, 2H), 6.59 (t, 1H), 5.82 (s, 1H), 3.81 (s, 3H), 3.28 (s, 8H) 2.42 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 515.145.

Compound-A8:4-(4-(7-chloroquinolin-4-yl)piperazin-1-yl)-N-(3-methoxyphenyl)-6-(trifluoromethyl)pyrimidin-2-amine: ¹H-NMR-400-MHz-DMSO: 9.43 (s, 1H), 8.70 (d, 1H), 8.45 (d, 1H), 7.74 (d, 1H), 7.38 (d, 1H) 7.21 (t, 1H), 6.79 (m, 2H), 6.59 (t, 1H), 5.82 (s, 1H), 3.81 (s, 3H), 3.28 (s, 8H) 2.42 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 514.937.

Compound-A9:4-(4-(7-chloroquinolin-4-yl)piperazin-1-yl)-N-(3-fluoro-5-methoxyphenyl)-6-(trifluoromethyl)pyrimidin-2-amine: ¹H-NMR-400 MHZ: 9.43 (s, 1H), 8.70 (d, 1H), 8.45 (d, 1H), 7.74 (d, 1H), 7.38 (d, 1H) 7.21 (t, 1H), 6.79 (m, 2H), 6.59 (t, 1H), 5.82 (s, 1H), 3.81 (s, 3H), 3.28 (s, 8H) 2.42 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 532.147.

Compound-A10:4-(4-(7-chloroquinolin-4-yl)piperazin-1-yl)-N-(3,5-dimethoxyphenyl)-6-methylpyridin-2-amine: ¹H-NMR-400-MHz-DMSO: 9.43 (s, 1H), 8.70 (d, 1H), 8.45 (d, 1H), 7.74 (d, 1H), 7.38 (d, 1H), 7.21 (t, 1H), 6.79 (m, 2H), 6.59 (t, 1H), 5.82 (s, 1H), 3.81 (s, 3H), 3.28 (s, 8H), 2.42 (s, 3H); ¹³C-NMR-400-MHz-DMSO: 160.6, 159.4, 159.2, 155.0, 153.0, 150.8, 144.4, 130.1, 128.9, 126.0, 122.6, 118.3, 98.9, 92.9, 91.7, 90.4, 55.8, 49.8, 48.5, 23.9; Molecularweight: 455.16.

Compound-A11: 7-chloro-4-(4-(6-methyl-2-(pyrrolidin-1-yl)pyrimidin-4-yl)piperazin-1-yl)quinoline: ¹H-NMR-400-MHz-DMSO: 8.74 (d, 1H), 8.15 (d, 1H), 8.01 (d, 1H), 7.59 (dd, 1H), 7.06 (d, 1H), 6.38 (s, 1H), 3.9 (s, 4H), 3.51 (d, 4H), 3.31 (s, 1H), 2.28 (s, 3H,) 1.93 (s, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 408.18.

Compound-A12:7-chloro-4-(4-(6-methyl-2-(piperidin-1-yl) pyrimidin-4-yl)piperazin-1-yl)quinoline: ¹H-NMR-400-MHz-DMSO: 8.73 (dd, 1H), 8.14 (q, 1H), 8.01 (dd, 1H), 7.59 (m, 1H), 7.10 (dd, 1H), 6.01 (s, 1H), 3.80 (s, 4H), 3.68 (t, 4H), 3.50 (s, 1H), 3.23 (t, 4H), 2.10 (s, 3H), 1.58 (t, 2H), 1.49 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 422.20.

Compound-A13:4-(4-(2-(azepan-1-yl)-6-methylpyrimidin-4-yl)piperazin-1-yl)-7-chloroquinoline: ¹H-NMR-400-MHz-DMSO: 8.36 (d, 1H), 8.23 (d, 1H), 7.87 (d, 1H), 7.36 (dd, 1H), 6.44 (d, 1H), 5.82 (d, 1H), 3.68 (t, 4H), 3.80 (s, 8H), 2.33 (s, 3H), 1.74 (t, 4H), 1.60 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 436.21.

Compound-A14:7-chloro-4-(4-(6-methyl-2-(piperazin-1-yl)pyrimidin-4-yl)piperazin-1-yl)quinoline: ¹H-NMR-400-MHz-DMSO: 8.36 (d, 1H), 8.23 (d, 1H), 7.87 (d, 1H), 7.36 (dd, 1H), 6.44 (d, 1H), 5.82 (d, 1H), 3.17 (t, 4H), 3.80 (s, 8H) 2.33 (s, 3H), 2.78 (t, 4H), 1.91 (s, 1H); ¹³C-NMR-400 MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 423.19.

Compound-A15:7-chloro-4-(4-(6-methyl-2-(4-methylpiperazin-1-yl)pyrimidin-4-yl)piperazin-1-yl)quinoline: ¹H-NMR-400-MHz-DMSO: 8.36 (d, 1H), 8.23 (d, 1H), 7.87 (d, 1H), 7.36 (dd, 1H), 6.44 (d, 1H), 5.82 (d, 1H), 3.15 (t, 4H), 3.37 (s, 8H) 2.33 (s, 3H), 2.36 (t, 4H), 2.26 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 437.21.

Compound-A16:4-(4-(4-(7-chloroquinolin-4-yl)piperazin-1-yl)-6-methylpyrimidin-2-yl)morpholine: ¹H-NMR-400-MHz-DMSO: 8.74 (d, 1H), 8.13 (dd, 1H), 8.00 (d, 1H), 7.58 (dd, 1H), 7.05 (d, 1H), 6.11 (s, 1H), 3.82 (s, 4H), 3.62 (s, 8H), 3.23 (t, 4H), 2.10 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 424.18.

Compound-A17:4-(4-(4-(7-chloroquinolin-4-yl)piperazin-1-yl)-6-methylpyrimidin-2-yl)thiomorpholine: ¹H-NMR-400-MHz-DMSO: 8.36 (d, 1H), 8.23 (d, 1H), 7.87 (d, 1H), 7.63 (d, 1H), 7.36 (dd, 1H), 6.44 (d, 1H), 5.82 (d, 1H), 3.43 (t, 4H), 3.87 (s, 8H) 2.33 (s, 3H), 2.67 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 440.15.

Compound-A18:4-(4-(7-chloroquinolin-4-yl)piperazin-1-yl)-N-cyclohexyl-6-methylpyrimidin-2-amine: ¹H-NMR-400-MHz-DMSO: 8.70 (d, 1H), 8.45 (d, 1H), 7.74 (s, 1H), 7.73 (d, 1H), 6.74 (d, 1H), 5.95 (s, 1H), 5.82 (s, 1H) 3.28 (t, 8H), 2.57 (p, 1H), 2.42 (s, 3H), 1.71 (m, 2H), 1.46 (m, 4H), 1.21 (m, 4H); ¹³C-NMR-400-MHz-DMSO: 170.2, 164.5, 160.6, 159.2, 153.0, 150.8, 135.6, 130.1, 128.9, 126.0, 122.6, 118.3, 93.8, 54.7, 49.8, 48.5, 32.9, 25.7, 25.1, 23.9; Molecularweight: 436.214.

Compound-A19:7-chloro-4-(4-(2-(pyrrolidin-1-yl)-6-(trifluoromethyl)pyrimidin-4-yl)piperazin-1-yl)quinoline: ¹H-NMR-400-MHz-DMSO: 8.73 (d, 1H), 8.14 (d, 1H), 8.01 (d, 1H), 7.59 (dd, 1H), 7.05 (d, 1H), 6.53 (s, 1H), 3.94 (s, 4H), 3.47 (s, 4H), 3.25 (t, 4H), 1.90 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 462.17.

Compound-A20:7-chloro-4-(4-(2-(piperidin-1-yl)-6-(trifluoromethyl)pyrimidin-4-yl)piperazin-1-yl)quinoline: ¹H-NMR-400-MHz-DMSO: 8.73 (d, 1H), 8.13 (d, 1H), 8.00 (d, 1H), 7.58 (dd, 1H), 7.05 (d, 1H), 6.52 (s, 1H), 3.93 (s, 4H), 3.71 (t, 4H), 3.26 (t, 4H), 1.61 (d, 2H), 1.52 (d, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 476.17.

Compound-A21:4-(4-(2-(azepan-1-yl)-6-(trifluoromethyl)pyrimidin-4-yl)piperazin-1-yl)-7-chloroquinoline: ¹H-NMR-400-MHz-DMSO: 8.73 (d, 1H), 8.13 (d, 1H), 8.01 (d, 1H), 7.58 (dd, 1H), 7.06 (d, 1H), 6.51 (s, 1H), 3.93 (s, 4H), 3.68 (t, 4H), 1.70 (s, 4H), 1.48 (t, 4H); ¹³C-NMR-400 MHZ-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 490.19.

Compound-A22:7-chloro-4-(4-(2-(piperazin-1-yl)-6-(trifluoromethyl)pyrimidin-4-yl)piperazin-1-yl)quinoline: ¹H-NMR-400-MHz-DMSO: 8.73 (d, 1H), 8.13 (dd, 1H), 8.01 (d, 1H), 7.58 (dd, 1H), 7.06 (d, 1H), 6.55 (s, 1H), 3.93 (s, 4H), 3.25 (t, 4H), 2.71 (t, 4H); ¹³C-NMR-400 MHZ-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 490.19.

Compound-A23:7-chloro-4-(4-(2-(4-methylpiperazin-1-yl)-6-(trifluoromethyl)pyrimidin-4-yl)piperazin-1-yl)quinoline: ¹H-NMR-400-MHz-DMSO: 8.73 (d, 1H), 8.13 (dd, 1H), 8.01 (d, 1H), 7.58 (dd, 1H), 7.06 (d, 1H), 6.58 (s, 1H), 3.94 (s, 4H), 3.70 (t, 4H), 3.26 (t, 4H), 2.34 (t, 4H), 2.20 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 490.18.

Compound-A24:4-(4-(4-(7-chloroquinolin-4-yl)piperazin-1-yl)-6-(trifluoromethyl)pyrimidin-2-yl)morpholine: ¹H-NMR-400-MHz-DMSO: 8.73 (d, 1H), 8.13 (dd, 1H), 8.01 (d, 1H), 7.59 (dd, 1H), 7.06 (d, 1H), 6.62 (s, 1H), 3.95 (s, 4H), 3.65 (m, 8H), 3.26 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 490.18.

Compound-A25:4-(4-(4-(7-chloroquinolin-4-yl)piperazin-1-yl)-6-(trifluoromethyl)pyrimidin-2-yl)thiomorpholine: ¹H-NMR-400-MHz-DMSO: 8.36 (d, 1H), 8.23 (d, 1H), 7.87 (d, 1H), 7.63 (d, 1H), 7.36 (dd, 1H), 6.44 (d, 1H), 5.82 (d, 1H), 3.43 (t, 4H), 3.67 (s, 8H) 2.67 (t, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 494.19.

Compound-A26:4-(4-(7-chloroquinolin-4-yl)piperazin-1-yl)-N-cyclohexyl-6-(trifluoromethyl)pyrimidin-2-amine: ¹H-NMR-400-MHz-DMSO: 8.70 (d, 1H), 8.45 (d, 1H), 7.74 (s, 1H), 7.73 (d, 1H), 6.74 (d, 1H), 5.95 (s, 1H), 5.82 (s, 1H), 4.00 (s, 1H), 3.28 (t, 8H), 2.57 (p, 1H), 1.71 (m, 2H), 1.46 (m, 4H), 1.21 (m, 4H); ¹³C-NMR-400-MHz-DMSO: 170.2, 160.5, 159.2, 154.7, 153.0, 150.8, 135.6, 130.1, 128.9, 126.0, 122.6, 119.7, 93.8, 54.7, 49.8, 48.5, 32.9, 25.7, 25.1; Molecularweight: 490.186.

Compound-A27:4-(4-(7-chloroquinolin-4-yl)piperazin-1-yl)-N-(2,6-difluorophenyl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 466.16

Compound-A28:4-(4-(7-chloroquinolin-4-yl)piperazin-1-yl)-N-(5-fluoropyridin-2-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400 MHZ-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 449.19.

Compound-A29:4-(4-(7-chloroquinolin-4-yl)piperazin-1-yl)-N-(pyridin-2-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 431.16.

Compound-A30:4-(4-(7-chloroquinolin-4-yl)piperazin-1-yl)-N-(pyridin-4-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 431.16.

Compound-A31:4-(4-(7-chloroquinolin-4-yl)piperazin-1-yl)-N-(2,3-dichlorophenyl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 498.02

Compound-A32: N-(3-bromophenyl)-4-(4-(7-chloroquinolin-4-yl)piperazin-1-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 6.64 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 508.08.

Compound-A33:4-(4-(7-chloroquinolin-4-yl)piperazin-1-yl)-N-(2,6-difluorophenyl)-6-(trifluoromethyl)pyrimidin-2-amine: ¹H-NMR-400-MHz-DMSO: 9.10 (s, 1H), 8.71 (d, 1H), 8.12 (d, 1H), 8.00 (d, 1H), 7.57 (dd, 1H), 7.29 (q, 1H), 7.14 (t, 1H), 7.04 (s, 2H), 6.74 (s, 1H), 3.86 (s, 4H), 3.21 (s, 4H); ¹³C-NMR-400 MHZ-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 520.12.

Compound-A34:4-(4-(7-chloroquinolin-4-yl)piperazin-1-yl)-N-(5-fluoropyridin-2-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.76 (dd, 2H), 4.00 (s, 1H), 3.90 (s, 8H), 3.83 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 503.12

Compound-A35:4-(4-(7-chloroquinolin-4-yl)piperazin-1-yl)-N-(pyridin-2-yl)-6-(trifluoromethyl)pyrimidin-2-amine: ¹H-NMR-400-MHz-DMSO: 8.36 (d, 1H), 8.23 (d, 1H), 8.07 (d, 1H), 7.87 (dd, 1H), 7.36 (d, 1H), 6.44 (d, 1H), 5.82 (d, 1H), 6.62 (d, 1H), 6.83 (d, 2H), 7.55 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 485.13.

Compound-A36:4-(4-(7-chloroquinolin-4-yl)piperazin-1-yl)-N-(pyridin-4-yl)-6-(trifluoromethyl)pyrimidin-2-amine: ¹H-NMR-400-MHz-DMSO: 10.08 (s, 1H), 8.74 (t, 1H), 8.36 (d, 1H), 8.17 (d, 1H), 8.02 (t, 1H), 7.70 (d, 2H), 7.61 (dd, 1H), 6.99 (d, 1H), 4.08 (s, 4H), 3.31 (s, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 485.13.

Compound-A37:4-(4-(7-chloroquinolin-4-yl)piperazin-1-yl)-N-(2,3-dichlorophenyl)-6-(trifluoromethyl)pyrimidin-2-amine: ¹H-NMR-400-MHz-DMSO: 8.36 (d, 1H), 8.23 (d, 1H), 7.87 (dd, 1H), 8.08 (d, 1H), 6.44 (d, 1H), 5.82 (d, 1H), 7.02 (d, 1H), 7.15 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 552.06.

Compound-A38: N-(3-bromophenyl)-4-(4-(7-chloroquinolin-4-yl)piperazin-1-yl)-6-(trifluoromethyl)pyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 6.94 (dd, 2H), 6.69 (d, 1H), 6.96 (d, 1H), 4.00 (s, 1H), 3.90 (s, 8H), 3.83 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 562.06.

Compound-A39:4-(4-(7-chloroquinolin-4-yl)piperazin-1-yl)-6-methyl-N-phenylpyrimidin-2-amine: ¹H-NMR-400-MHz-DMSO: 9.02 (s, 1H), 8.74 (d, 1H), 8.16 (d, 1H), 8.01 (d, 1H), 7.73 (dd, 1H), 7.23 (q, 2H), 7.07 (d, 1H), 6.87 (m, 1H), 6.24 (s, 1H), 3.89 (s, 4H), 3.28 (s, 4H), 2.23 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 430.17.

Compound-A40:4-(4-(7-chloroquinolin-4-yl)piperazin-1-yl)-N-phenyl-6-(trifluoromethyl)pyrimidin-2-amine: ¹H-NMR-400-MHz-DMSO: 8.74 (d, 1H), 8.16 (d, 1H), 8.01 (d, 1H), 7.73 (dd, 2H), 7.60 (dd, 1H), 7.28 (m, 1H), 7.07 (d, 1H), 6.93 (t, 1H), 6.76 (s, 1H), 4.01 (s, 4H), 3.29 (s, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 484.14.

Compound-A41:4-(4-(7-chloroquinolin-4-yl)piperazin-1-yl)-N-(3,5-dimethoxyphenyl)pyrimidin-2-amine: ¹H-NMR-400-MHz-DMSO: 9.05 (s, 1H), 8.73 (d, 1H), 8.16 (d, 1H), 8.02 (q, 2H), 7.60 (dd, 1H), 7.05 (t, 3H), 6.36 (d, 1H), 6.06 (s, 1H), 3.92 (s, 4H), 3.70 (s, 6H), 3.30 (d, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 476.17.

Compound-A42: N-(3,5-dimethoxyphenyl)-4-methyl-6-(4-(quinolin-4-yl)piperazin-1-yl)pyrimidin-2-amine: ¹H-NMR-400-MHz-DMSO: 9.05 (s, 1H), 8.71 (d, 1H), 8.16 (d, 1H), 7.97 (dd, 1H), 7.76 (t, 1H), 7.60 (m, 1H), 7.07 (t, 3H), 6.26 (s, 1H), 6.04 (t, 1H), 3.92 (s, 4H), 3.71 (d, 6H), 3.32 (s, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 456.23.

Compound-A43: N-(3,5-dimethoxyphenyl)-4-(4-(quinolin-4-yl)piperazin-1-yl)-6-(trifluoromethyl)pyrimidin-2-amine: ¹H-NMR-400-MHz-DMSO: 8.36 (d, 1H), 8.17 (d, 1H), 7.92 (t, 1H), 7.76 (dd, 1H), 7.59 (t, 1H), 6.44 (d, 1H), 5.82 (d, 1H), 5.73 (s, 2H), 5.74 (d, 1H), 4.00 (s, 1H), 3.83 (s, 6H), 3.28 (s, 8H), 3.32 (s, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 456.23.

Compound-A44: N-(3,5-dimethoxyphenyl)-4-methyl-6-(4-(7-(trifluoromethyl)quinolin-4-yl)piperazin-1-yl)pyrimidin-2-amine: ¹H-NMR-400-MHz-DMSO: 10.03 (s, 1H), 8.82 (d, 1H), 8.47 (d, 1H), 8.38 (s, 1H), 7.93 (d, 1H), 7.24 (d, 1H), 6.79 (s, 1H), 6.60 (s, 1H), 6.29 (s, 1H), 4.00 (d, 8H), 3.73 (s, 6H), 2.35 (s, 3H); ¹³C-NMR-400-MHz-DMSO: 170.2, 168.0, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9; Molecularweight: 524.21.

Compound-A45: N-(3,5-dimethoxyphenyl)-4-(trifluoromethyl)-6-(4-(7-(trifluoromethyl)quinolin-4-yl)piperazin-1-yl)pyrimidin-2-amine: ¹H-NMR-400-MHz-DMSO: 10.03 (s, 1H), 8.82 (d, 1H), 8.47 (d, 1H), 8.38 (s, 1H), 7.93 (d, 1H), 7.24 (d, 1H), 6.79 (s, 2H), 6.60 (s, 1H), 6.29 (s, 1H), 4.00 (d, 8H), 3.73 (s, 6H), 2.35 (s, 3H); ¹³C-NMR-400-MHz-DMSO: 170.2, 168.0, 160.6, 157.6, 158.2, 149.2, 144.4, 135.4, 129.5, 128.4, 124.0, 121.1, 111.8, 93.8, 91.7, 90.4, 55.8, 49.8, 48.5; Molecularweight: 578.19.

Compound-A46: N-(3,5-dimethoxyphenyl)-4-methyl-6-(4-(quinazolin-4-yl)piperazin-1-yl)pyrimidin-2-amine: ¹H-NMR-400-MHz-DMSO: 8.49 (s, 1H), 8.16 (d, 1H), 7.84 (t, 1H), 7.83 (t, 1H), 7.58 (t, 1H), 5.82 (d, 1H), 5.73 (s, 2H), 5.74 (d, 1H), 4.00 (s, 1H), 3.83 (s, 6H), 3.28 (s, 8H), 2.33 (s, 3H); ¹³C-NMR-400-MHz-DMSO: 179.6, 170.2, 168.0, 164.5, 160.6, 156.6, 151.4, 144.4, 132.7, 129.5, 127.4, 122.5, 93.8, 91.7, 90.4, 55.8, 49.8, 48.5, 23.9; Molecularweight: 457.22.

Compound-A47: N-(3,5-dimethoxyphenyl)-4-(4-(quinazolin-4-yl)piperazin-1-yl)-6-(trifluoromethyl)pyrimidin-2-amine: ¹H-NMR-400-MHz-DMSO: 8.49 (s, 1H), 8.16 (d, 1H), 7.84 (t, 1H), 7.83 (t, 1H), 7.58 (t, 1H), 5.82 (d, 1H), 5.73 (s, 2H), 5.74 (d, 1H), 4.00 (s, 1H), 3.83 (s, 6H), 3.28 (s, 8H); ¹³C-NMR-400-MHz-DMSO: 179.6, 170.2, 168.0, 160.6, 156.6, 154.7, 151.7, 144.4, 131.9, 127.4, 122.2, 119.7, 93.8, 91.7, 90.4, 55.8, 49.8, 48.5; Molecularweight: 511.19.

Compound-A48: N-(3,5-dimethoxyphenyl)-4-(4-(7-fluoroquinolin-4-yl)piperazin-1-yl)-6-methylpyrimidin-2-amine: ¹H-NMR-400-MHz-DMSO: 11.94 (s, 1H), 9.06 (s, 1H), 8.66 (s, 1H), 8.12 (d, 1H), 7.83 (q, 2H), 7.58 (m, 1H), 7.07 (d, 2H), 6.02 (s, 1H), 6.04 (s, 1H), 3.89 (s, 8H), 3.70 (s, 6H), 2.22 (s, 3H), 1.90 (s, 2H); ¹³C-NMR-400-MHz-DMSO: 170.2, 168.0, 164.5, 163.8, 160.6, 159.4, 153.0, 149.5, 144.4, 127.5, 124.9, 128.4, 117.4, 116.7, 113.2, 93.8, 91.7, 90.4, 55.8, 49.8, 48.5, 23.9; Molecularweight: 511.19.

Compound-A49: N-(3,5-dimethoxyphenyl)-4-(4-(7-fluoroquinolin-4-yl)piperazin-1-yl)-6-(trifluoromethyl)pyrimidin-2-amine: ¹H-NMR-400-MHz-DMSO: 9.56 (s, 1H), 8.73 (d, 1H), 8.21 (q, 1H), 7.70 (dd, 1H), 7.51 (m, 1H), 7.03 (q, 4H), 6.79 (s, 1H), 6.11 (t, 1H), 5.75 (s, 1H), 4.04 (s, 4H), 3.70 (s, 6H), 3.39 (q, 2H), 1.09 (t, 3H); ¹³C-NMR-400-MHz-DMSO: 170.2, 168.0, 163.8, 160.6, 159.4, 154.7, 153.0, 149.5, 144.4, 127.5, 124.9, 119.7, 117.4, 113.2, 93.8, 91.7, 90.4, 55.8, 49.8, 48.5; Molecularweight: 528.19.

Compound-A50:4-(4-(7-chloroquinolin-4-yl)piperazin-1-yl)-N-(3,5-dimethoxyphenyl)-6-(trifluoromethyl)pyridin-2-amine: ¹H-NMR-400-MHz-DMSO: 8.36 (d, 1H), 8.23 (d, 1H), 7.87 (s, 1H), 7.36 (d, 1H), 6.44 (d, 1H), 5.77 (d, 1H), 5.73 (s, 2H), 5.74 (d, 1H), 4.00 (s, 1H), 3.83 (s, 6H), 3.28 (s, 8H); ¹³C-NMR-400-MHz-DMSO: 160.6, 159.4, 159.2, 155.0, 153.0, 150.8, 144.4, 147.3, 135.6, 130.1, 128.6, 126.0, 122.6, 119.7, 118.3, 98.9, 92.9, 91.7, 90.4, 55.8, 49.8, 48.5; Molecularweight: 543.16.

Compound-A51:4-(4-(7-chloroquinolin-4-yl)piperazin-1-yl)-N-(3,5-dimethoxyphenyl)-6-methylpyridin-2-amine: ¹H-NMR-400-MHz-DMSO: 8.36 (d, 1H), 8.23 (d, 1H), 7.87 (s, 1H), 7.36 (d, 1H), 6.44 (d, 1H), 5.77 (d, 1H), 5.73 (s, 2H), 5.74 (d, 1H), 4.00 (s, 1H), 3.83 (s, 6H), 3.28 (s, 8H), 2.33 (s, 3H); ¹³C-NMR-400-MHz-DMSO: 160.6, 159.4, 159.2, 155.0, 153.0, 150.8, 144.4, 130.1, 128.9, 126.0, 122.6, 118.3, 98.9, 92.9, 91.7, 90.4, 55.8, 49.8, 48.5, 23.9; Molecularweight: 489.19.

Compound-A52:4-(4-(7-chloroquinolin-4-yl)piperazin-1-yl)-N-(3,5-dimethoxyphenyl)-6-fluoropyrimidin-2-amine: ¹H-NMR-400-MHz-DMSO: 8.01 (d, 1H), 7.62 (d, 1H), 7.57 (d, 1H), 7.32 (dd, 1H), 7.38 (d, 1H), 6.98 (d, 1H), 5.74 (s, 1H), 5.74 (s, 1H), 5.73 (s, 1H), 5.26 (d, 1H), 3.83 (s, 6H), 4.00 (s, 1H), 3.57 (t, 4H), 3.28; ¹³C-NMR-400-MHz-DMSO: 176.1, 166.9, 160.6, 159.4, 159.2, 153.0, 150.8, 144.4, 135.6, 130.1, 128.9, 126.0, 122.6, 118.3, 91.7, 90.4, 83.0, 55.8, 49.8, 48.5; Molecular weight: 494.19.

Compound-A53:4-(4-(7-chloroquinolin-4-yl)piperazin-1-yl)-N-(3,5-dimethoxyphenyl)-5-fluoro-6-methylpyrimidin-2-amine: ¹H-NMR-400-MHz-DMSO: 8.36 (d, 1H), 8.23 (d, 1H), 7.87 (d, 1H), 7.36 (dd, 1H), 6.44 (d, 1H), 5.74 (s, 2H), 5.73 (s, 1H), 4.00 (s, 1H), 3.90 (s, 8H), 3.83 (s, 6H), 2.33 (s, 3H); ¹³C-NMR-400-MHz-DMSO: 164.1, 160.6, 159.2, 157.2, 153.0, 150.8, 130.0, 135.6, 130.1, 118.3, 122.6, 118.3, 93.8, 90.4, 55.8, 49.8, 48.5, 15.3; Molecular weight: 508.18.

Compound-A54:4-(4-(7-chloroquinolin-4-yl)piperazin-1-yl)-N-(2,6-difluoro-3,5-dimethoxyphenyl)-6-methylpyrimidin-2-amine: ¹H-NMR-400-MHz-DMSO: 8.36 (d, 1H), 8.23 (d, 1H), 7.87 (s, 1H), 7.36 (d, 1H), 6.44 (d, 1H), 5.82 (d, 1H), 5.90 (d, 1H), 4.00 (s, 1H), 3.30 (s, 8H), 3.83 (s, 6H), 2.33 (s, 3H); ¹³C-NMR-400 MHZ-DMSO: 170.2, 168.0, 164.5, 163.0, 159.2, 158.5, 153.0, 150.8, 145.6, 135.6, 130.1, 128.9, 126.0, 122.6, 118.3, 100.4, 94.7, 93.4, 92.4, 55.8, 49.8, 48.5, 23.9; Molecular weight: 526.17.

Compound-A55: 4-(4-(6,7-difluoroquinolin-4-yl)piperazin-1-yl)-N-(3,5-dimethoxyphenyl)-6-methylpyrimidin-2-amine: ¹H-NMR-400-MHz-DMSO: 8.36 (d, 1H), 7.68 (d, 1H), 7.36 (dd, 1H), 6.44 (d, 1H), 5.74 (d, 2H), 5.73 (d, 1H), 5.82 (d, 1H), 4.00 (s, 1H), 3.90 (s, 8H), 3.83 (s, 6H), 2.33 (s, 3H); ¹³C-NMR-400 MHZ-DMSO: 170.2, 168.0, 164.5, 160.6, 158.8, 154.4, 152.3, 151.3, 146.5, 144.4, 125.4, 118.4, 115.6, 109.4, 93.8, 91.7, 90.4, 55.8, 49.8, 48.5, 23.9; Molecular weight: 526.17.

Compound-A56:4-(4-(7-chloro-6-fluoroquinolin-4-yl)piperazin-1-yl)-N-(3,5-dimethoxyphenyl)-6-methylpyrimidin-2-amine: ¹H-NMR-400-MHz-DMSO: 8.36 (d, 1H), 7.85 (d, 1H), 7.32 (dd, 1H), 6.44 (d, 1H), 5.74 (d, 2H), 5.73 (d, 1H), 5.82 (d, 1H), 4.00 (s, 1H), 3.90 (s, 8H), 3.83 (s, 6H), 2.33 (s, 3H); ¹³C-NMR-400 MHZ-DMSO: 170.2, 168.0, 164.5, 161.7, 160.6, 158.6, 152.3, 147.8, 144.4, 132.5, 126.5, 125.7, 119.4, 108.2, 93.8, 91.7, 90.4, 55.8, 49.8, 48.5, 23.9; Molecular weight: 508.18.

Compound-A57: 4-(4-(7-chloro-4a,5,6,7,8,8a-hexahydroquinolin-4-yl)piperazin-1-yl)-N-(3,5-dimethoxyphenyl)-6-methylpyrimidin-2-amine: ¹H-NMR-400-MHz-DMSO: 7.50 (d, 1H), 5.82 (d, 1H), 5.74 (d, 2H), 5.73 (d, 1H), 4.96 (m, 1H), 4.49 (d, 1H), 4.00 (s, 1H), 3.83 (s, 6H), 3.65 (t, 4H), 3.42 (m, 3H), 2.78 (t, 4H), 2.33 (s, 3H), 2.10 (q, 1H), 1.70 (m, 2H), 1.60 (m, 2H), 1.40 (m, 2H): ¹³C-NMR-400-MHz-DMSO: 170.2, 168.0, 164.5, 162.1, 160.6, 144.4, 93.8, 91.7, 90.4, 93.8, 59.6, 55.8, 53.7, 53.5, 47.0, 37.9, 36.2, 23.9, 19.2; Molecular weight: 496.24.

ompound-A58:4-(4-(7-chloro-2-methyl-4a,5,6,7,8,8a-hexahydroquinolin-4-yl)piperazin-1-yl)-N-(3,5-dimethoxyphenyl)-6-methylpyrimidin-2-amine: ¹H-NMR-400-MHz-DMSO: 5.82 (d, 1H), 5.74 (d, 2H), 5.73 (d, 1H), 4.96 (m, 1H), 4.49 (d, 1H), 4.00 (s, 1H), 3.83 (s, 6H), 3.65 (t, 4H), 3.42 (m, 3H), 2.78 (t, 4H), 2.33 (s, 3H), 2.10 (q, 1H), 2.07 (s, 3H), 1.70 (m, 2H), 1.60 (m, 2H), 1.40 (m, 2H); ¹³C-NMR-400-MHz-DMSO: 170.2, 168.0, 164.5, 162.1, 160.6, 144.4, 93.8, 91.7, 90.4, 93.8, 59.6, 55.8, 53.7, 53.5, 47.0, 37.9, 36.2, 23.9, 19.2, 18.7; Molecular weight: 510.4.

Compound-A59: 4-(4-(7-chloro-2-(difluoromethyl)-4a,5,6,7,8,8a-hexahydroquinolin-4-yl)piperazin-1-yl)-N-(3,5-dimethoxyphenyl)-6-methylpyrimidin-2-amine: ¹H-NMR-400-MHz-DMSO: 6.50 (t, 1H), 5.82 (d, 2H), 5.74 (d, 2H), 5.73 (d, 1H), 4.96 (m, 1H), 4.49 (d, 1H), 4.00 (s, 1H), 3.83 (s, 6H), 3.65 (t, 4H), 3.42 (m, 3H), 2.78 (t, 4H), 2.33 (s, 3H), 2.10 (q, 1H), 1.70 (m, 2H), 1.60 (m, 2H), 1.40 (m, 2H); ¹³C-NMR-400-MHz-DMSO: 170.2, 168.0, 164.5, 164.6, 162.1, 160.6, 144.4, 122.4, 95.5, 93.8, 91.7, 59.6, 55.8, 53.0, 38.2, 37.5, 36.5, 23.9, 19.2; Molecular weight: 531.21.

Compound-A60:4-(4-(7-chlorodecahydroquinolin-4-yl)piperazin-1-yl)-N-(3,5-dimethoxyphenyl)-6-methylpyrimidin-2-amine: ¹H-NMR-400-MHz-DMSO: 5.82 (s, 1H), 5.74 (d, 2H), 5.73 (d, 1H), 4.00 (s, 1H), 3.83 (s, 6H), 3.42 (m, 1H), 2.84 (s, 8H), 2.79 (m, 2H), 2.62 (q, 1H), 2.56 (q, 1H), 2.33 (s, 3H), 2.00 (t, 1H), 1.89 (d, 2H), 1.70 (m, 2H), 1.65 (m, 2H), 1.49 (m, 2H), 1.32 (m, 2H); ¹³C-NMR-400-MHz-DMSO: 170.2, 168.0, 164.5, 164.6, 160.6, 144.4, 93.8, 90.4, 68.7, 55.8, 54.1, 53.1, 48.7, 48.2, 41.8, 32.0, 28.8, 24.7, 18.9; Molecular weight: 500.27.

Compound-A61:4-(4-(7-chloroquinolin-4-yl)piperazin-1-yl)-N-(3,5-dimethoxycyclohex-1-en-1-yl)-6-methylpyrimidin-2-amine; ¹H-NMR-400-MHz-DMSO: 8.36 (d, 1H), 8.23 (d, 1H), 7.70 (dd, 1H), 7.70 (dd, 1H), 7.13 (d, 1H), 6.44 (d, 1H), 5.06 (d, 1H), 5.82 (d, 1H), 4.00 (s, 1H), 3.90 (s, 8H), 3.31 (d, 1H), 3.30 (d, 1H), 3.30 (s, 6H), 2.33 (s, 3H), 2.00 (m, 2H), 2.11 (m, 2H); ¹³C-NMR-400-MHz-DMSO: 170.2, 168.0, 164.5, 164.6, 160.6, 144.4, 93.8, 90.4, 68.7, 55.8, 54.1, 53.1, 48.7, 48.2, 41.8, 32.0, 28.8, 24.7, 18.9; Molecular weight: 500.27.

Compound-A62: 5-((4-(4-(7-chloroquinolin-4-yl)piperazin-1-yl)-6-methylpyrimidin-2-yl)amino)cyclohexane-1,3-diol: ¹H-NMR-400-MHz-DMSO: 8.36 (d, 1H), 8.23 (d, 1H), 7.87 (s, 1H), 7.36 (dd, 1H), 7.13 (d, 1H), 6.44 (d, 1H), 5.82 (d, 1H), 4.00 (s, 1H), 3.90 (s, 8H), 3.31 (d, 1H), 3.30 (d, 1H), 2.33 (s, 3H), 2.57 (d, 1H), 1.72 (m, 2H), 1.74 (m, 2H), 1.68 (m, 2H); ¹³C-NMR-400-MHz-DMSO: 170.2, 164.5, 160.6, 159.2, 135.6, 130.6, 128.6, 126.0, 122.2, 118.6, 93.8, 61.7, 49.8, 45.5, 55.8, 54.1, 53.1, 48.7, 48.2, 40.2, 32.0, 28.8, 24.7, 23.9S; Molecular weight: 468.20.

Compound-A63:4-(4-(7-chloroquinolin-4-yl)piperazin-1-yl)-N-(3,5-dimethylcyclohexyl)-6-methylpyrimidin-2-amine: ¹H-NMR-400-MHz-DMSO: 8.36 (d, 1H), 8.23 (d, 1H), 7.87 (dd, 1H), 7.36 (d, 1H), 6.44 (d, 1H), 5.82 (d, 1H), 4.00 (s, 1H), 3.90 (s, 8H), 3.31 (d, 1H), 3.30 (d, 1H), 2.33 (s, 3H), 2.57 (d, 1H), 1.72 (m, 2H), 1.74 (m, 2H), 1.68 (m, 2H), 0.96 (s, 6H); ¹³C-NMR-400-MHz-DMSO: 170.2, 164.5, 160.6, 159.2, 153.0, 150.8, 128.6, 126.0, 122.6, 118.6, 93.8, 48.5, 44.0, 43.4, 28.1, 23.9, 21.0; Molecular weight: 464.28.

Compound-A64:4-(4-(7-chloroquinolin-4-yl)piperazin-1-yl)-6-methyl-N-(3-methylenecyclohexyl)pyrimidin-2-amine: ¹H-NMR-400 MHz-DMSO: 8.36 (d, 1H), 8.23 (d, 1H), 7.87 (d, 1H), 7.30 (d, 1H), 6.44 (d, 1H), 5.82 (d, 1H), 5.11 (s, 8H), 4.00 (s, 1H), 3.90 (s, 8H), 2.65 (m, 1H), 2.33 (s, 3H), 2.02 (d, 1H), 1.95 (m, 2H), 1.45 (m, 2H), 1.34 (m, 2H); ¹³C-NMR-400 MHZ-DMSO: 170.2, 164.5, 160.6, 159.2, 153.0, 150.8, 148.9, 135.6, 130.0, 128.6, 126.0, 122.6, 118.3, 107.8, 93.8, 69.9, 49.8, 48.5, 42.5, 35.7, 31.4, 23.9; Molecular weight: 448.21.

Compound-A65: 3-((4-(4-(7-chloroquinolin-4-yl)piperazin-1-yl)-6-methylpyrimidin-2-yl)amino)cyclohexanone: ¹H-NMR-400-MHz-DMSO: 8.36 (d, 1H), 8.23 (d, 1H), 7.87 (d, 1H), 7.36 (d, 1H), 6.44 (d, 1H), 5.82 (d, 1H), 4.00 (s, 1H), 3.90 (s, 8H), 2.95 (m, 1H), 2.33 (s, 3H), 2.20 (m, 2H), 2.10 (m, 2H), 2.00 (m, 2H), 2.08 (m, 2H), 1.79 (m, 2H); ¹³C-NMR-400-MHz-DMSO: 210.8, 170.2, 164.5, 160.6, 159.2, 153.0, 150.8, 148.9, 135.6, 130.0, 128.6, 126.0, 122.6, 118.3, 107.8, 93.8, 69.9, 49.8, 48.5, 42.5, 35.7, 31.4, 23.9; Molecular weight: 450.19.

Compound-A66: 2-((4-(4-(7-chloroquinolin-4-yl)piperazin-1-yl)-6-methylpyrimidin-2-yl)amino)cyclohexa-2,5-diene-1,4-dione: ¹H-NMR-400-MHz-DMSO: 8.01 (d, 1H), 7.62 (d, 1H), 7.57 (s, 1H), 7.38 (d, 1H), 7.32 (d, 2H), 6.87 (s, 2H), 6.25 (s, 1H), 5.82 (s, 1H), 4.00 (s, 1H), 3.90 (s, 8H), 2.33 (s, 3H); ¹³C-NMR-400-MHz-DMSO: 185.7, 181.7, 170.2, 164.5, 160.6, 159.2, 153.0, 153.3, 150.8, 148.9, 136.8, 135.6, 136.8, 128.0, 122.6, 118.3, 107.8, 93.8, 69.9, 49.8, 48.5, 42.5, 35.7, 31.4, 23.9; Molecular weight: 460.16.

Compound-A67:4-(4-(7-chloro-2-methylquinolin-4-yl)piperazin-1-yl)-N-(3,5-dimethoxyphenyl)-6-methylpyrimidin-2-amine: ¹H-NMR-40 MHz-DMSO: 8.24 (d, 1H), 7.84 (s, 1H), 7.32 (d, 1H), 6.66 (d, 1H), 5.82 (d, 2H), 5.74 (s, 2H), 5.73 (s, 1H), 4.00 (s, 1H), 3.83 (s, 6H), 3.90 (s, 8H), 2.33 (s, 3H), 2.53 (s, 3H); ¹³C-NMR-400-MHz-DMSO: 170.2, 164.1, 160.6, 158.2, 149.2, 144.4, 135.4, 129.5, 128.4, 124.0, 121.1, 111.8, 93.8, 91.7, 90.4, 55.8, 49.8, 48.5, 23.9, 20.0; Molecular weight: 504.20.

Compound-A68:4-(4-(7-chloro-2,8-dimethylquinolin-4-yl)piperazin-1-yl)-N-(3,5-dimethoxyphenyl)-6-methylpyrimidin-2-amine: ¹H-NMR-400-MHz-DMSO: 7.86 (d, 1H), 7.58 (s, 1H), 7.15 (s, 1H), 6.38 (s, 1H), 5.82 (d, 1H), 5.74 (s, 2H), 5.73 (s, 1H), 4.00 (s, 1H), 3.28 (s, 8H), 3.83 (s, 6H), 2.64 (s, 3H), 2.45 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 518.44.

Compound-A69:4-(4-(7-chloro-2,3-dimethylquinolin-4-yl)piperazin-1-yl)-N-(3,5-dimethoxyphenyl)-6-methylpyrimidin-2-amine: ¹H-NMR-400-MHz-DMSO: 7.86 (d, 1H), 7.58 (s, 1H), 7.15 (s, 1H), 6.38 (s, 1H), 5.82 (d, 1H), 5.74 (s, 2H), 5.73 (s, 1H), 4.00 (s, 1H), 3.90 (s, 8H), 3.83 (s, 6H), 2.45 (s, 3H), 2.23 (s, 3H), 2.31 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 504.20.

Compound-A70:4-(4-(7-chloroquinolin-4-yl)-2,6-dimethylpiperazin-1-yl)-N-(3,5-dimethoxyphenyl)-6-methylpyrimidin-2-amine: ¹H-NMR-400-MHz-DMSO: 8.01 (d, 1H), 7.62 (d, 1H), 7.57 (s, 1H), 7.38 (d, 1H), 7.32 (d, 2H), 6.98 (d, 2H), 5.74 (d, 2H), 5.73 (d, 1H), 5.82 (d, 1H), 4.00 (s, 1H), 3.38 (s, 4H), 3.03 (m, 2H), 3.83 (s, 6H), 2.33 (s, 3H), 1.12 (s, 6H). ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 518.22.

Compound-A71:4-(7-(7-chloroquinolin-4-yl)-4,7-diazaspiro[2.5]octan-4-yl)-N-(3,5-dimethoxyphenyl)-6-methylpyrimidin-2-amine: ¹H-NMR-400-MHz-DMSO: 8.01 (d, 1H), 7.62 (d, 1H), 7.57 (s, 1H), 7.38 (d, 1H), 7.32 (d, 2H), 6.98 (d, 2H), 5.74 (d, 2H), 5.73 (d, 1H), 5.82 (d, 1H), 4.00 (s, 1H), 3.38 (s, 6H), 3.83 (s, 6H), 2.33 (s, 3H), 0.35 (m, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 516.02.

Compound-A72:4-(4-(7-chloro-2-methylquinolin-4-yl)-2-methylpiperazin-1-yl)-N-(3,5-dimethoxyphenyl)-6-methylpyrimidin-2-amine: ¹H-NMR-400-MHz-DMSO: 8.02 (d, 1H), 7.59 (dd, 1H), 7.48 (d, 1H), 7.28 (d, 1H), 6.41 (s, 1H), 5.74 (d, 2H), 5.73 (d, 1H), 5.82 (d, 1H), 4.00 (s, 1H), 3.83 (s, 6H), 3.38 (m, 2H), 3.01 (m, 6H), 3.03 (d, 1H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 518.22.

Compound-A73:4-(4-(7-chloroquinolin-4-yl)piperazin-1-yl)-N-(3,5-dimethoxy-4-methylphenyl)-5,6-dimethylpyrimidin-2-amine: ¹H-NMR-400-MHz-DMSO: 8.01 (d, 1H), 7.62 (d, 1H), 7.57 (s, 1H), 7.38 (d, 1H), 7.32 (d, 2H), 6.98 (d, 2H), 5.62 (d, 2H), 4.00 (s, 1H), 3.83 (s, 6H), 3.38 (m, 8H), 2.33 (s, 6H); ¹³C-NMR-400-MHz-DMSO: 170.4, 165.0, 164.5, 160.4, 159.2, 153.0, 149.2, 141.4, 135.4, 129.5, 128.4, 124.0, 121.1, 111.8, 93.8, 91.7, 90.4, 55.8, 49.8, 48.5, 23.9, 20.0, 16.6, 18.8, 15.9, 9.5; Molecular weight: 518.22.

Compound-A74:4-(4-(7-chloroquinolin-4-yl)piperazin-1-yl)-N-(3,5-dimethylphenyl)-5,6-dimethylpyrimidin-2-amine: ¹H-NMR-400-MHz-DMSO: 8.01 (d, 1H), 7.62 (d, 1H), 7.57 (s, 1H), 7.38 (d, 1H), 7.32 (d, 2H), 6.98 (d, 2H), 5.62 (d, 2H), 4.00 (s, 1H), 3.38 (m, 8H), 2.34 (s, 6H), 2.33 (s, 6H); ¹³C-NMR-400-MHz-DMSO: 170.2, 165.4, 159.2, 163.0, 150.8, 142.2, 139.1, 135.6, 130.1, 128.9, 126.0, 122.6, 120.6, 118.6, 118.0, 98.9, 91.7, 49.8, 48.5, 21.6, 18.0, 15.9; Molecular weight: 472.11.

Compound-A75:4-(4-(7-chloro-2-methylquinolin-4-yl)-3-methylpiperazin-1-yl)-N-(3,5-dimethylphenyl)-5,6-dimethylpyrimidin-2-amine: ¹H-NMR-400-MHz-DMSO: 8.02 (d, 1H), 7.59 (d, 2H), 7.48 (dd, 1H), 7.28 (d, 1H), 6.71 (d, 1H), 6.41 (d, 2H), 4.00 (s, 1H), 3.38 (m, 6H), 3.03 (d, 1H), 2.34 (s, 6H), 2.33 (s, 6H), 2.45 (s, 6H), 1.22 (s, 3H); ¹³C-NMR-400-MHz-DMSO: 170.4, 165.7, 165.4, 160.5, 158.4, 149.4, 142.4, 139.1, 129.5, 128.4, 124.0, 121.5, 118.0, 118.8, 98.6, 91.7, 90.4, 65.8, 52.8, 49.1, 48.5, 21.6, 20.0, 18.0, 16.3; Molecular weight: 500.25.

Compound-A76:4-(4-(7-chloroquinolin-4-yl)piperazin-1-yl)-N-(3-ethoxy-5-methoxyphenyl)-6-methylpyrimidin-2-amine: ¹H-NMR-400-MHz-DMSO: 8.02 (d, 1H), 7.62 (d, 2H), 7.57 (dd, 1H), 7.38 (d, 1H), 7.32 (d, 1H), 6.98 (d, 1H), 6.37 (s, 1H), 5.74 (s, 2H), 5.73 (s, 1H), 5.82 (d, 1H), 4.09 (q, 2H), 4.00 (s, 1H), 3.38 (s, 8H), 3.83 (d, 3H), 2.33 (s, 6H) 1.32 (s, 3H); ¹³C-NMR-400-MHz-DMSO: 170.2, 168.1, 164.5, 160.2, 159.2, 153.0, 150.8, 144.0, 135.6, 130.1, 128.9, 126.0, 126.3, 118.3, 93.8, 91.8, 91.0, 64.6, 55.8, 49.5, 48.5, 23.9, 14.8; Molecular weight: 504.20.

Compound-A77: 4-(4-(7-chloroquinolin-4-yl)piperazin-1-yl)-N-(3,5-diethoxyphenyl)-6-methylpyrimidin-2-amine: ¹H-NMR-400-MHz-DMSO: 8.02 (d, 1H), 7.62 (d, 2H), 7.57 (dd, 1H), 7.38 (d, 1H), 7.32 (d, 1H), 6.98 (d, 1H), 6.37 (s, 1H), 5.74 (s, 2H), 5.73 (s, 1H), 5.82 (d, 1H), 4.09 (q, 4H), 4.00 (s, 1H), 3.38 (s, 8H), 3.83 (d, 3H), 2.33 (s, 6H) 1.32 (s, 6H); ¹³C-NMR-400-MHz-DMSO: 170.2, 168.1, 164.5, 160.6, 159.2, 158.8, 153.0, 150.8, 143.4, 135.6, 130.1, 128.9, 126.0, 122.6, 118.3, 93.8, 91.1, 64.6, 49.8, 48.5, 14.8; Molecular weight: 518.22.

Compound-A78:4-(4-(2,7-dichloro-3-methoxyquinolin-4-yl)piperazin-1-yl)-N-(3,5-dimethoxyphenyl)-6-methylpyrimidin-2-amine: ¹H-NMR-400 MHz-DMSO: 8.16 (d, 1H), 7.84 (d, 1H), 7.36 (m, 1H), 5.82 (d, 1H), 5.74 (s, 2H), 5.73 (s, 1H), 4.00 (s, 1H), 3.38 (s, 8H), 3.83 (s, 9H), 2.53 (s, 3H), 2.33 (s, 3H); ¹³C-NMR-400-MHz-DMSO: 170.2, 168.1, 164.5, 160.6, 159.2, 158.8, 153.0, 150.8, 143.4, 135.6, 130.1, 128.9, 126.0, 122.6, 118.3, 93.8, 91.1, 64.6, 49.8, 48.5, 14.8; Molecular weight: 554.16.

Compound-A79:4-(4-(7-chloro-6-methoxyquinolin-4-yl)piperazin-1-yl)-N-(3,5-dimethoxyphenyl)-6-methylpyrimidin-2-amine: ¹H-NMR-400-MHz-DMSO: 8.25 (d, 1H), 7.80 (s, 1H), 7.10 (s, 1H), 6.44 (d, 1H), 5.82 (d, 1H), 5.74 (s, 2H), 5.73 (s, 1H), 4.00 (s, 1H), 3.38 (s, 8H), 3.83 (s, 9H), 2.53 (s, 3H), 2.53 (s, 3H), 2.31 (s, 3H); ¹³C-NMR-400-MHz-DMSO: 170.2, 168.1, 164.5, 160.6, 158.6, 150.6, 146.5, 144.4, 131.6, 126.6, 128.9, 126.0, 122.6, 118.3, 104.1, 93.8, 91.7, 90.4, 55.8, 49.8, 48.5, 14.8; Molecular weight: 520.20.

Compound-A80:4-(4-(7-chloro-8-methoxyquinolin-4-yl)piperazin-1-yl)-N-(3,5-dimethoxyphenyl)-6-methylpyrimidin-2-amine: ¹H-NMR-400-MHz-DMSO: 7.86 (d, 1H), 7.50 (d, 1H), 6.70 (s, 1H), 5.82 (d, 1H), 5.74 (s, 2H), 5.73 (s, 1H), 4.00 (s, 1H), 3.38 (s, 8H), 3.83 (s, 9H), 2.53 (s, 3H), 2.53 (s, 3H), 2.31 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 520.20.

Compound-A81: N-(3,5-dimethoxyphenyl)-4-(4-(7-methoxyquinolin-4-yl)piperazin-1-yl)-6-methylpyrimidin-2-amine: ¹H-NMR-400-MHz-DMSO: 8.36 (d, 1H), 7.98 (d, 1H), 7.27 (d, 1H), 7.24 (d, 1H), 6.33 (d, 1H), 5.82 (d, 1H), 5.74 (s, 2H), 5.73 (s, 1H), 4.00 (s, 1H), 3.38 (s, 8H), 3.83 (s, 9H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 486.24.

Compound-A82: N-(3,5-dimethoxyphenyl)-4-(4-(6,7-dimethoxyquinolin-4-yl)piperazin-1-yl)-6-methylpyrimidin-2-amine: ¹H-NMR-400 MHZ-DMSO: 8.25 (d, 1H), 7.20 (s, 1H), 7.09 (d, 1H), 6.33 (d, 1H), 5.82 (d, 1H), 5.74 (s, 2H), 5.73 (s, 1H), 4.00 (s, 1H), 3.38 (s, 8H), 3.83 (s, 12H), 2.33 (s, 3H); ¹³C-NMR-400 MHZ-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 516.25.

Compound-A83: N-(3,5-dimethoxyphenyl)-4-methyl-6-(4-(5,6,7-trimethoxyquinolin-4-yl)piperazin-1-yl)pyrimidin-2-amine: ¹H-NMR-400-MHz-DMSO: 8.29 (d, 1H), 6.82 (s, 1H), 6.36 (dd, 1H), 5.82 (d, 1H), 5.74 (s, 2H), 5.73 (s, 1H), 4.00 (s, 1H), 3.38 (s, 8H), 3.83 (s, 16H), 2.33 (s, 3H); ¹³C-NMR-400-MHz-DMSO: 170.2, 168.0, 164.5, 160.6, 157.6, 158.2, 150.5, 146.3, 144.4, 128.4, 115.0, 114.6, 99.7, 93.8, 91.7, 60.7, 56.1, 55.8, 49.8, 48.5, 23.9; Molecular weight: 546.26.

Compound-A84:4-(4-(6,7-diethoxy-5-methoxyquinolin-4-yl)piperazin-1-yl)-N-(3,5-dimethoxyphenyl)-6-methylpyrimidin-2-amine: ¹H-NMR-400-MHz-DMSO: 8.29 (d, 1H), 6.82 (s, 1H), 6.36 (dd, 1H), 5.82 (d, 1H), 5.74 (s, 2H), 5.73 (s, 1H), 4.00 (s, 1H), 3.38 (s, 8H), 3.83 (s, 9H), 2.33 (s, 3H), 1.33 (s, 6H); ¹³C-NMR-400-MHz-DMSO: 170.2, 168.0, 164.5, 160.6, 156.8, 158.2, 150.5, 158.4, 146.3, 144.4, 135.4, 129.5, 128.4, 124.0, 121.1, 115.593.8, 91.7, 90.4, 55.8, 49.8, 48.5, 23.9, 14.8; Molecular weight: 574.29.

Compound-A85: N-(3,5-diethoxyphenyl)-4-(4-(6,7-diethoxyquinolin-4-yl)piperazin-1-yl)-6-methylpyrimidin-2-amine: ¹H-NMR-400-MHz-DMSO: 8.25 (d, 1H), 7.20 (s, 1H), 7.09 (s, 1H), 6.33 (d, 1H), 5.82 (d, 1H), 5.74 (s, 2H), 5.73 (s, 1H), 4.09 (m, 8H) 4.00 (s, 1H), 3.38 (s, 8H), 2.33 (s, 3H) 1.33 (s, 12H).); ¹³C-NMR-400-MHz-DMSO: 170.2, 168.0, 164.5, 158.5, 157.9, 153.4, 150.2, 145.5, 124.5, 116.7, 107.6, 104.7, 93.8, 91.1, 90.6, 64.6, 49.8, 48.5, 23.9, 14.8; Molecular weight: 572.31.

Compound-A86: N-(3,5-dimethoxyphenyl)-4-(4-(6,7-dimethoxyquinazolin-4-yl)piperazin-1-yl)-6-methylpyrimidin-2-amine: ¹H-NMR-400-MHz-DMSO: 8.49 (d, 1H), 7.41 (s, 1H), 7.24 (s, 1H), 5.82 (d, 1H), 5.74 (s, 2H), 5.73 (s, 1H), 4.00 (s, 1H), 3.38 (s, 8H), 3.83 (s, 9H), 2.33 (s, 3H).); ¹³C-NMR-400-MHz-DMSO: 178.1, 170.2, 168.1, 164.5, 160.6, 159.2, 154.7, 152.3, 148.1, 119.2, 108.293.8, 91.7, 90.4, 55.8, 49.8, 48.5, 23.9, 20.0, 16.6; Molecular weight: 517.24.

Compound-A87:4-(4-(7-chloro-1,8-naphthyridin-4-yl)piperazin-1-yl)-N-(3,5-dimethoxyphenyl)-6-methylpyrimidin-2-amine: ¹H-NMR-400-MHz-DMSO: 8.68 (d, 1H), 8.55 (d, 1H), 7.77 (d, 1H), 6.71 (d, 1H), 5.82 (d, 1H), 5.74 (s, 2H), 5.73 (s, 1H), 4.00 (s, 1H), 3.38 (s, 8H), 3.83 (s, 6H), 2.33 (s, 3H); ¹³C-NMR-400-MHz-DMSO: 170.2, 168.1, 164.5, 160.6, 156.6, 153.2, 148.1, 144.4, 137.3, 122.4, 120.4, 109.0, 93.8, 91.7, 90.4, 55.8, 49.8, 48.5, 23.9; Molecular weight: 491.24.

Compound-A88:4-(4-(7-chloro-1,5-naphthyridin-4-yl)piperazin-1-yl)-N-(3,5-dimethoxyphenyl)-6-methylpyrimidin-2-amine: ¹H-NMR-400-MHz-DMSO: 9.19 (d, 1H), 8.95 (dd, 1H), 8.65 (d, 1H), 6.86 (d, 1H), 5.82 (d, 1H), 5.74 (s, 2H), 5.73 (s, 1H), 4.00 (s, 1H), 3.38 (s, 8H), 3.83 (s, 6H), 2.33 (s, 3H); ¹³C-NMR-400-MHz-DMSO: 170.2, 168.0, 164.5, 160.6, 155.3, 150.2, 148.2, 144.4, 135.5, 127.6, 124.3, 109.3, 93.8, 91.7, 90.4, 55.8, 49.8, 48.5, 23.9; Molecular weight: 491.18.

Compound-A89:4-(3-(7-chloroquinolin-4-yl)imidazolidin-1-yl)-N-(3,5-dimethoxyphenyl)-6-methylpyrimidin-2-amine: ¹H-NMR-400-MHz-DMSO: 8.36 (d, 1H), 8.23 (d, 1H), 7.37 (d, 2H), 7.87 (d, 2H), 6.44 (d, 1H), 5.82 (d, 1H), 5.74 (s, 2H), 5.73 (s, 1H), 4.00 (s, 1H), 4.83 (s, 2H), 3.29 (s, 4H), 3.83 (s, 6H), 2.33 (s, 3H); ¹³C-NMR-400-MHz-DMSO: 167.0, 168.1, 160.6, 159.2, 153.0, 150.8, 149.2, 144.4, 130.1, 126.0, 122.6, 118.0, 93.8, 91.7, 90.4, 55.8, 49.8, 48.5, 23.9, 20.0, 16.6; Molecular weight: 476.17.

Compound-A90:4-(3-(7-chloroquinazolin-4-yl)imidazolidin-1-yl)-N-(3,5-dimethoxyphenyl)-6-methylpyrimidin-2-amine: ¹H-NMR-400-MHz-DMSO: 8.49 (s, 1H), 8.02 (d, 1H), 7.87 (d, 1H), 7.78 (d, 1H), 6.44 (d, 2H), 5.82 (d, 1H), 5.74 (s, 2H), 5.73 (s, 1H), 4.00 (s, 1H), 4.83 (s, 2H), 3.29 (s, 4H), 3.83 (s, 6H), 2.33 (s, 3H).); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 477.17.

Compound-A91:4-(3-(7-chloro-1,8-naphthyridin-4-yl)imidazolidin-1-yl)-N-(3,5-dimethoxyphenyl)-6-methylpyrimidin-2-amine: ¹H-NMR-400-MHz-DMSO: 8.68 (s, 1H), 8.55 (d, 1H), 7.77 (d, 1H), 6.71 (d, 1H), 5.82 (d, 1H), 5.74 (s, 2H), 5.73 (s, 1H), 4.00 (s, 1H), 4.83 (s, 2H), 3.29 (s, 4H), 3.83 (s, 6H), 2.33 (s, 3H).); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 477.17.

Compound-A92:4-(4-(7-chloroquinolin-4-yl)-1,4-diazepan-1-yl)-N-(3,5-dimethoxyphenyl)-6-methylpyrimidin-2-amine: ¹H-NMR-400-MHz-DMSO: 8.36 (d, 1H), 8.23 (d, 1H), 7.87 (d, 1H), 7.36 (d, 1H), 6.44 (d, 1H), 5.82 (d, 1H), 5.74 (s, 2H), 5.73 (s, 1H), 4.00 (s, 1H), 3.68 (t, 4H), 3.29 (s, 4H), 3.83 (s, 6H), 2.33 (s, 3H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 504.20.

Compound-A93:4-(4-(7-chloroquinazolin-4-yl)-1,4-diazepan-1-yl)-N-(3,5-dimethoxyphenyl)-6-methylpyrimidin-2-amine: ¹H-NMR-400-MHz-DMSO: 8.49 (s, 1H), 8.02 (s, 1H), 7.87 (d, 1H), 7.78 (d, 1H), 6.44 (d, 1H), 5.82 (d, 1H), 5.74 (s, 2H), 5.73 (s, 1H), 4.00 (s, 1H), 3.68 (t, 4H), 3.29 (s, 4H), 3.83 (s, 6H), 2.33 (s, 3H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 504.20.

Compound-A94:4-(4-(7-chloro-1,8-naphthyridin-4-yl)-1,4-diazepan-1-yl)-N-(3,5-dimethoxyphenyl)-6-methylpyrimidin-2-amine: ¹H-NMR-400-MHz-DMSO: 8.68 (d, 1H), 8.55 (d, 1H), 7.77 (d, 1H), 6.71 (d, 1H), 5.82 (d, 1H), 5.74 (s, 2H), 5.73 (s, 1H), 4.00 (s, 1H), 4.83 (s, 2H), 3.29 (s, 4H), 3.83 (s, 6H), 2.33 (s, 3H) 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 504.20.

Compound-A95: 4-(4-(7-chloro-1,5-naphthyridin-4-yl)-1,4-diazepan-1-yl)-N-(3,5-dimethoxyphenyl)-6-methylpyrimidin-2-amine: ¹H-NMR-400-MHz-DMSO: 9.19 (s, 1H), 8.95 (d, 1H), 8.65 (d, 1H), 6.86 (d, 1H), 5.82 (d, 1H), 5.74 (s, 2H), 5.73 (s, 1H), 4.00 (s, 1H), 3.68 (t, 4H), 3.29 (s, 4H), 3.83 (s, 6H), 2.33 (s, 3H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 505.20.

Compound-A96:4-(4-(7-chloro-1,5-naphthyridin-4-yl)-1,4-diazepan-1-yl)-N-(3,5-dimethoxyphenyl)-6-(trifluoromethyl)pyrimidin-2-amine: ¹H-NMR-400-MHz-DMSO: 9.19 (s, 1H), 8.95 (d, 1H), 8.65 (s, 1H), 6.84 (d, 1H), 5.82 (d, 1H), 5.74 (s, 2H), 5.73 (s, 1H), 4.00 (s, 1H), 3.68 (t, 4H), 3.29 (s, 4H), 3.83 (s, 6H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 559.19.

Compound-A97: 4-(4-(7-chloro-4a,5,6,7,8,8a-hexahydroquinolin-4-yl)piperazin-1-yl)-N-(3-ethoxy-5-methoxyphenyl)-6-fluoropyrimidin-2-amine: ¹H-NMR-400-MHz-DMSO: 7.50 (d, 1H), 5.26 (d, 1H), 5.74 (d, 2H), 5.73 (d, 1H), 4.96 (m, 1H), 4.49 (d, 1H), 4.09 (m, 1H), 4.00 (s, 1H), 3.83 (s, 3H), 3.65 (t, 4H), 3.42 (m, 3H), 2.78 (t, 4H), 2.33 (s, 3H), 2.10 (q, 1H), 1.70 (m, 2H), 1.60 (m, 2H), 1.40 (m, 2H) 1.32 33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 514.23.

Compound-A98:4-(7-(7-chloroquinolin-4-yl)-4,7-diazaspiro[2.5]octan-4-yl)-N-(3,5-dimethoxycyclohex-1-en-1-yl)-6-(trifluoromethyl)pyrimidin-2-amine: ¹H-NMR-400-MHz-DMSO: 8.36 (d, 1H), 8.23 (d, 1H), 7.87 (d, 1H), 7.36 (d, 1H), 6.44 (d, 1H), 5.74 (d, 2H), 5.73 (d, 1H), 5.82 (d, 1H), 4.00 (s, 1H), 3.30 (s, 6H), 3.28 (s, 4H), 3.21 (s, 4H), 3.31 (m, 1H), 3.00 (m, 4H), 2.10 (m, 2H), 2.00 (m, 2H), 2.33 (s, 3H), 1.12 (s, 3H) 0.35 (m, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 514.23.

Compound-A99:4-(4-(2,7-dichloro-3-methoxyquinolin-4-yl)piperazin-1-yl)-N-(2,6-difluoro-3,5-dimethoxyphenyl)-6-methylpyrimidin-2-amine: ¹H-NMR-400-MHz-DMSO: 8.16 (d, 1H), 7.84 (s, 1H), 7.36 (d, 1H), 7.36 (d, 1H), 5.90 (t, 1H), 5.82 (s, 1H), 5.98 (d, 1H), 4.00 (s, 1H), 3.30 (s, 8H), 3.83 (s, 9H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 590.14.

Compound-A100:2-((4-(4-(7-chloro-6-methoxyquinolin-4-yl)-2-methylpiperazin-1-yl)-5,6-dimethylpyrimidin-2-yl)amino)cyclohexa-2,5-diene-1,4-dione: ¹H-NMR-400-MHz-DMSO: 8.25 (d, 1H), 7.80 (s, 1H), 7.10 (s, 1H), 6.87 (s, 2H), 6.44 (d, 1H), 6.25 (s, 1H), 5.82 (d, 1H), 4.00 (s, 1H), 3.90 (s, 8H), 3.83 (s, 3H), 3.22 (m, 2H), 3.00 (d, 1H) 3.01 (m, 2H), 2.90 (m, 2H) 2.33 (s, 3H) 2.34 (s, 3H) 1.12 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 518.18.

Compound-A101: N-(3,5-diethoxyphenyl)-4-(3-(6,7-dimethoxyquinazolin-4-yl)imidazolidin-1-yl)-6-methylpyrimidin-2-amine: ¹H-NMR-400-MHz-DMSO: 8.49 (s, 1H), 7.41 (s, 1H), 7.24 (s, 2H), 5.74 (s, 2H), 5.73 (s, 1H), 5.82 (d, 1H), 4.83 (s, 2H), 4.09 (q, 4H), 4.00 (s, 1H), 3.29 (s, 4H), 3.83 (d, 3H), 2.33 (s, 6H) 1.32 (s, 6H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 531.26.

Compound-A102:3-(7-chloro-1,8-naphthyridin-4-yl)-2-cyclopropyl-1-(2-((3,5-dihydroxycyclohexyl)amino)-6-methylpyrimidin-4-yl)imidazolidin-4-one: ¹H-NMR-400-MHz-DMSO: 8.68 (d, 1H), 8.65 (d, 1H), 7.77 (dd, 1H), 7.76 (d, 1H), 5.82 (d, 1H), 4.71 (d, 1H), 3.95 (d, 2H), 4.00 (s, 1H), 3.90 (s, 8H), 3.31 (d, 1H), 3.58 (s, 2H), 3.17 (m, 2H), 2.57 (m, 1H), 2.46 (m, 1H), 2.33 (s, 3H), 1.46 (m, 4H), 1.68 (m, 4H), 1.07 (m, 1H), 0.30 (m, 4H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 509.19.

Compound-A103:4-(3-(7-chloro-8-methoxyquinolin-4-yl)imidazolidin-1-yl)-N-(3-fluoro-5-methoxyphenyl)-6-methylpyrimidin-2-amine: ¹H-NMR-400-MHz-DMSO: 8.39 (s, 1H), 7.85 (d, 1H), 7.54 (d, 2H), 7.32 (dd, 1H), 6.50 (d, 2H), 6.48 (d, 2H), 5.95 (d, 1H), 5.82 (d, 1H), 4.83 (s, 2H), 4.00 (s, 1H), 3.29 (s, 4H), 3.83 (d, 9H), 2.33 (s, 6H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 494.16.

Compound-A104: N-(3,5-dimethoxyphenyl)-4,5-dimethyl-6-(4-(5,6,7-trimethoxyquinazolin-4-yl)-1,4-diazepan-1-yl)pyrimidin-2-amine: ¹H-NMR-400-MHz-DMSO: 8.49 (s, 1H), 6.97 (s, 1H), 5.74 (s, 2H), 5.73 (s, 1H), 4.00 (s, 1H), 3.68 (t, 4H), 3.29 (s, 4H), 3.83 (s, 16H), 2.33 (s, 3H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 575.29.

Compound-A105:4-(4-(7-chloro-2-(thiophen-2-yl)-1,5-naphthyridin-4-yl)-1,4-diazepan-1-yl)-N-(3,5-dimethoxyphenyl)-6-methylpyrimidin-2-amine: ¹H-NMR-400-MHz-DMSO: 9.19 (s, 1H), 8.65 (s, 1H), 7.85 (d, 1H), 7.69 (d, 1H), 7.17 (d, 1H), 5.82 (d, 1H), 5.74 (s, 2H), 5.73 (s, 1H), 4.00 (s, 1H), 3.68 (t, 4H), 3.29 (s, 4H), 3.83 (s, 6H), 1.75 (m, 2H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 587.19.

Compound-A106: 3-((4-(4-(7-chloroquinolin-4-yl)piperazin-1-yl)-6-methylpyrimidin-2-yl)amino)-5-methoxyphenol: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 5.90 (d, 1H), 5.74 (d, 1H), 5.35 (s, 1H), 3.83 (s, 3H), 4.00 (s, 1H), 3.28 (s, 8H) 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 476.17.

Compound-A107:3-((4-(4-(7-chloroquinolin-4-yl)piperazin-1-yl)-6-(trifluoromethyl)pyrimidin-2-yl)amino)-5-methoxyphenol: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 5.90 (d, 1H), 5.74 (d, 1H), 5.35 (s, 1H), 3.83 (s, 3H), 4.00 (s, 1H), 3.28 (s, 8H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 530.17.

Compound-A108:5-((4-(4-(7-chloroquinolin-4-yl)piperazin-1-yl)-6-methylpyrimidin-2-yl)amino)benzene-1,3-diol: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 5.90 (d, 1H), 5.74 (d, 1H), 5.35 (s, 2H), 4.00 (s, 1H), 3.28 (s, 8H) 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 462.16.

Compound-A109:5-((4-(4-(7-chloroquinolin-4-yl)piperazin-1-yl)-6-(trifluoromethyl)pyrimidin-2-yl)amino)benzene-1,3-diol: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 5.90 (d, 1H), 5.74 (d, 1H), 5.35 (s, 2H), 4.00 (s, 1H), 3.28 (s, 8H)); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 516.17.

Compound-A110: N1-(4-(4-(7-chloroquinolin-4-yl)piperazin-1-yl)-6-methylpyrimidin-2-yl)-5-methoxy-N3,N3-dimethylbenzene-1,3-diamine: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 5.52 (d, 1H), 5.46 (d, 1H), 5.67 (s, 1H), 3.83 (s, 3H), 3.06 (s, 6H), 4.00 (s, 1H), 3.28 (s, 8H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 503.22.

Compound-A111: N1-(4-(4-(7-chloroquinolin-4-yl)piperazin-1-yl)-6-methylpyrimidin-2-yl)-5-methoxybenzene-1,3-diamine: ¹H-NMR-400-MHz-DMSO: 8.36 (d, 1H), 8.23 (d, 1H), 7.87 (d, 1H), 7.36 (d, 1H) 6.44 (d, 1H), 6.27 (s, 2H), 5.82 (d, 1H), 5.52 (d, 1H), 5.46 (d, 1H), 5.67 (s, 1H), 3.83 (s, 3H), 3.06 (s, 6H), 4.00 (s, 1H), 3.28 (s, 8H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 475.19.

Compound-A112: 3-((4-(4-(7-chloroquinolin-4-yl)piperazin-1-yl)-6-methylpyrimidin-2-yl)amino)-5-methoxybenzonitrile: ¹H-NMR-400-MHz-DMSO: 8.36 (d, 1H), 8.23 (d, 1H), 7.87 (d, 1H), 7.36 (d, 1H) 6.44 (d, 1H), 6.27 (s, 2H), 5.82 (d, 1H), 6.97 (t, 1H), 6.46 (dd, 1H), 6.28 (t, 1H), 3.83 (s, 3H), 4.00 (s, 1H), 3.28 (s, 8H) 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecular weight: 475.19.

Compound-A113:3-((4-(4-(7-chloroquinolin-4-yl)piperazin-1-yl)-6-(trifluoromethyl)pyrimidin-2-yl)amino)-5-methoxybenzonitrile: ¹H-NMR-400-MHz-DMSO: 8.36 (d, 1H), 8.23 (d, 1H), 7.87 (d, 1H), 7.36 (d, 1H) 6.44 (d, 1H), 6.27 (s, 2H), 5.82 (d, 1H), 6.97 (t, 1H), 6.46 (dd, 1H), 6.28 (t, 1H), 3.83 (s, 3H), 4.00 (s, 1H), 3.28 (s, 8H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 539.14.

Compound-A114:3-((4-(4-(7-chloroquinolin-4-yl)piperazin-1-yl)-6-(trifluoromethyl)pyrimidin-2-yl)amino)-5-hydroxybenzonitrile: ¹H-NMR-400-MHz-DMSO: 8.36 (d, 1H), 8.23 (d, 1H), 7.87 (d, 1H), 7.36 (d, 1H) 6.44 (d, 1H), 6.27 (s, 2H), 5.82 (d, 1H), 5.35 (s, 1H) 6.93 (t, 1H), 6.42 (dd, 1H), 6.28 (t, 1H), 4.00 (s, 1H), 3.28 (s, 8H); ¹³C-NMR-40-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 525.13.

Compound-A115:1-(3-((4-(4-(7-chloroquinolin-4-yl)piperazin-1-yl)-6-methylpyrimidin-2-yl)amino)-5-methoxyphenyl)ethanone: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 6.85 (t, 1H), 6.37 (t, 1H), 6.56 (d, 1H), 3.83 (s, 3H), 4.00 (s, 1H), 3.28 (s, 8H) 2.50 (s, 3H), 2.33 (s, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 502.13.

Compound-A116:1-(3-((4-(4-(7-chloroquinolin-4-yl)piperazin-1-yl)-6-methylpyrimidin-2-yl)amino)-5-methoxyphenyl)ethanol: ¹H-NMR-400-MHz-DMSO: 8.36 (d, 1H), 8.23 (d, 1H), 7.87 (d, 1H), 7.36 (d, 1H) 6.44 (d, 1H), 5.82 (d, 1H), 6.50 (d, 1H), 6.24 (s, 1H), 6.11 (d, 1H), 3.65 (s, 1H), 3.83 (s, 3H), 4.68 (t, 1H) 4.00 (s, 1H), 3.28 (s, 8H), 2.33 (s, 3H) 1.49 (d, 3H); ¹³C-NMR-400-MHz-DMSO: 170.2, 168.1, 164.5, 160.6, 157.6, 158.2, 149.2, 144.4, 135.4, 129.5, 128.4, 124.0, 121.1, 111.8, 93.8, 91.7, 90.4, 55.8, 49.8, 48.5, 23.9, 20.0, 16.6; Molecularweight: 502.13.

### Example 2

### Experiments Related to Test Anti-Cancer Properties of Compounds A1-A116

### Cells Used for Testing Anti-Cancer Properties of the Compounds

The following human derived non-small cell lung cancer cell lines were used to test the efficiency of the compounds to cause cell death and assess the anti-cancer properties of said compounds: 1. A549 cells: adenocarcinomic human alveolar basal epithelial cells; these cells are used as models for the study of lung cancer and the development of drug therapies against it
2. NCI-H-460 cells are also human lung cancer cells, and
3. NCI-H-522 cells are also human lung cancer cells.

### Cell Cytotoxicity Assay Protocol

500 cells/well/30 μL were seeded in a 384 well cell culture plate. Incubated over night at 37° C., 5% CO2 in cell culture incubator. 10 μL of 4× compound was added to the plates, and incubated for 72 hours at 37° C., 5% CO2 in cell culture incubator. Assay DMSO control were included for 0% cytotoxicity control. Assay was terminated by addition of 10 μL of the reagent CTG 2.0 detection reagent #G9242. Plates were incubated for 20 minute incubation at room temperature with shaking Plate were read in Envision. Ultrasensitive luminescence and data was analyzed and percent cytotoxicity of compounds was determined against DMSO control.

### CellTiter Glo Assay Protocol

Cells were cultured and maintained in F12K+10% FBS+1× anti-anti media and RPMI+10% FBS+1× anti-anti media. Cells were trypsinised and expanded to ensure that cells are not over-confluent and are in log growth phase. On day 0, 500 cells/well were seeded in 30 μl media using cell culture treated white flat bottom 384 well plate, centrifuged at 40×g for 10 seconds and incubated overnight in 370 C, 5% CO2 incubator. On day 1, 4× concentration of compounds were prepared in complete media, 10 μl was added to cells, centrifuged at 40×g for 10 seconds and incubated 72 hours in 37° C., 5% CO₂ incubator. Compounds tested: 100 μM top, 3 fold dilution and 10 point DRC in triplicate. Final DMSO concentration was maintained at 0.5% for all compound. No compound/DMSO was used as a control. On day 4 (72 hours post treatment) plates were brought to room temperature for 10 min 10 μl of CellTiter Glo reagent was added to each well and placed the plates on an orbital shaker for 10 minutes at room temperature. Luminescence signal was read on the Envision plate reader using the ultra-sensitive luminescence protocol. Data was plotted and analyzed using the below formula: DMSO control treated cells RLU considered as 100% cell viability.

$$Percent luminescence = ( ( sample RLU ) / ( average DMSO RLU ) ) * 100$$

### Cancerous Cell Toxicity Assay Results Derivatives of Formula A

All the compounds, A1-A116, were tested for cell toxicity using said non-small cell lung cancer cell lines using the above described method. Table 6 provides cell toxicity results of derivatives of Formula A which showed most efficient toxicity against cancerous cells of all the tested derivatives of Formula A.

TABLE 6
Cell toxicity results of most efficient derivatives of Formula A

| | A549 cells | | NCI-H-460 cells | | NCI-H-522 cells | |
|---|---|---|---|---|---|---|
| Compound | 10 μM | 1 μM | 10 μM | 1 μM | 10 μM | 1 μM |
| A1 | 97 | 42 | 82 | 19 | 50 | 21 |
| A2 | 1 | 9 | −3 | −8 | 14 | 4 |
| A4 | 95 | 47 | 71 | −4 | 57 | 14 |
| A5 | 5 | 24 | 7 | −8 | 22 | 6 |
| A6 | 4 | 15 | −1 | −7 | 21 | 7 |
| A7 | 65 | 53 | 25 | −1 | 42 | 18 |
| A8 | 5 | 14 | −3 | −9 | 7 | 1 |
| A11 | 10 | 16 | −4 | −12 | 9 | 2 |
| A12 | 99 | 10 | 99 | −9 | 99 | 5 |
| A13 | 88 | 14 | 47 | −11 | 48 | 3 |
| A16 | −17 | 9 | −6 | −11 | 12 | 8 |
| A19 | 87 | 25 | 45 | −8 | 48 | 11 |
| A20 | 89 | 37 | 60 | −10 | 43 | 8 |
| A21 | 23 | 13 | −8 | −15 | 9 | 1 |
| A22 | 17 | 11 | −2 | −14 | 11 | 1 |
| A23 | 13 | 12 | −2 | −15 | 9 | 1 |
| A24 | 22 | −1 | −6 | −9 | 8 | 0 |
| A33 | −8 | 8 | 3 | −9 | 17 | 2 |
| A36 | 99 | 0 | 98 | −11 | 72 | 2 |
| A39 | 18 | 4 | −4 | −11 | 15 | 1 |
| A40 | 8 | 15 | −7 | −12 | 11 | 6 |
| A10 | 100 | 88 | 72 | 12 | 79 | 41 |
| A41 | 42 | 23 | 28 | 2 | 56 | 7 |
| A42 | 98 | 68 | 67 | 4 | 80 | 20 |
| A43 | 99 | 88 | 65 | 8 | 73 | 48 |
| A44 | 18 | 26 | 7 | 0 | 26 | 8 |
| A45 | 83 | 8 | 12 | 0 | 55 | 7 |
| A46 | 98 | 25 | 44 | −2 | 66 | 2 |
| A47 | 44 | 38 | 12 | 4 | 43 | 45 |
| A48 | 98 | 57 | 66 | 0 | 71 | 16 |
| A49 | 99 | 64 | 59 | 3 | 77 | 45 |

This clearly shows that Compounds A1, A2, A4, A5, A6, A7, A8, A11, A12, A13, A16, A19, A20, A21, A22, A23, A24, A33, A36, A39, A40, A41, A42, A43, A44, A45, A46, A47, A48, A49 are active against Non-small cell lung cancer, whereas other tested derivatives of Formula A has lower efficient cell toxicity properties against Non-small cell lung cancer cell lines. These selected derivatives of Formula A were further tested to confirm their efficiency as anti-cancer compounds.

Cell Titre Glo assay results for selected derivatives of Formula A Compounds A1, A2, A4, A5, A6, A7, A8, A11, A12, A13, A16, A19, A20, A21, A22, A23, A24, A33, A36, A39, A40, A41, A42, A43, A44, A45, A46, A47, A48, A49 which showed maximum efficiency in cell toxicity assay were further tested for cell Titer Glo assay. Of the selected compounds only A1, A7, A42, A43, A48, and A49 showed best efficiency in cell toxicity against Non-small cell lung cancer cell lines. Table 7 provides the IC50 values for cell toxicity of most efficient derivatives of Formula A.

TABLE 7
Cell Titre Glo assay results of selected derivatives of Formula A

| | Cytotoxicity IC50 (μM) | | |
|---|---|---|---|
| Compound | A549 | NCI-H-460 | NCI-H-522 |
| A1 | 1.588 | 2.412 | 3.035 |
| A7 | 4.191 | 3.419 | 2.564 |
| A42 | 1.009 | 2.354 | 4.209 |
| A43 | 0.665 | 0.943 | 1.230 |
| A48 | 0.818 | 1.839 | 3.237 |
| A49 | 0.576 | 0.842 | 1.836 |

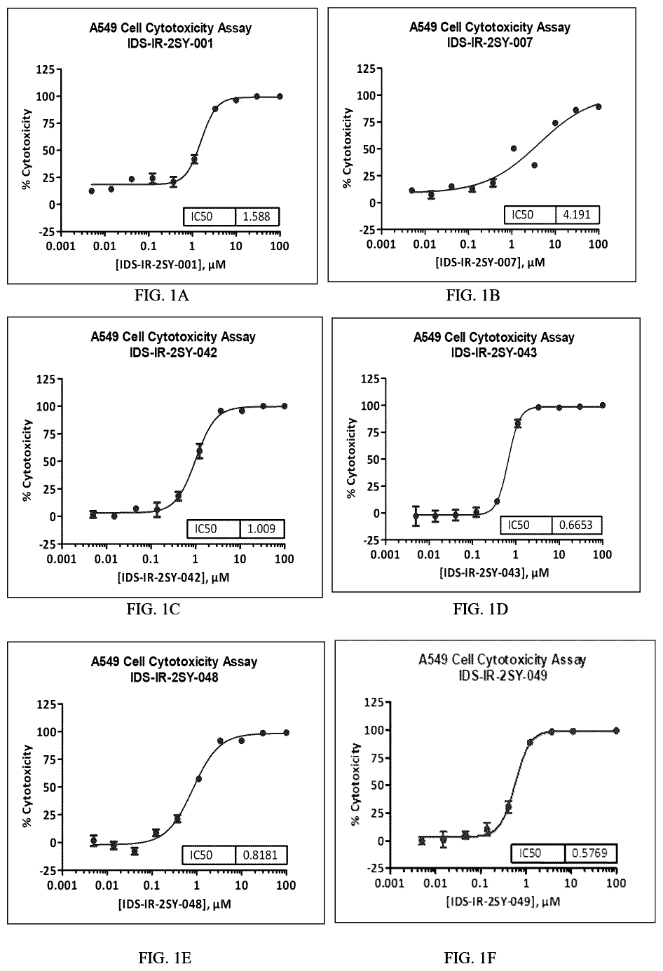

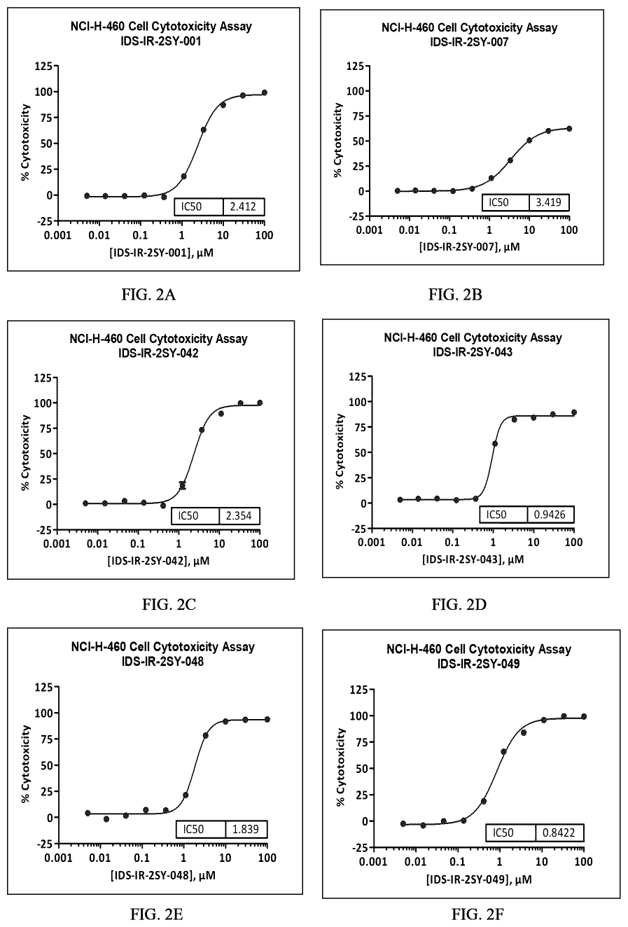

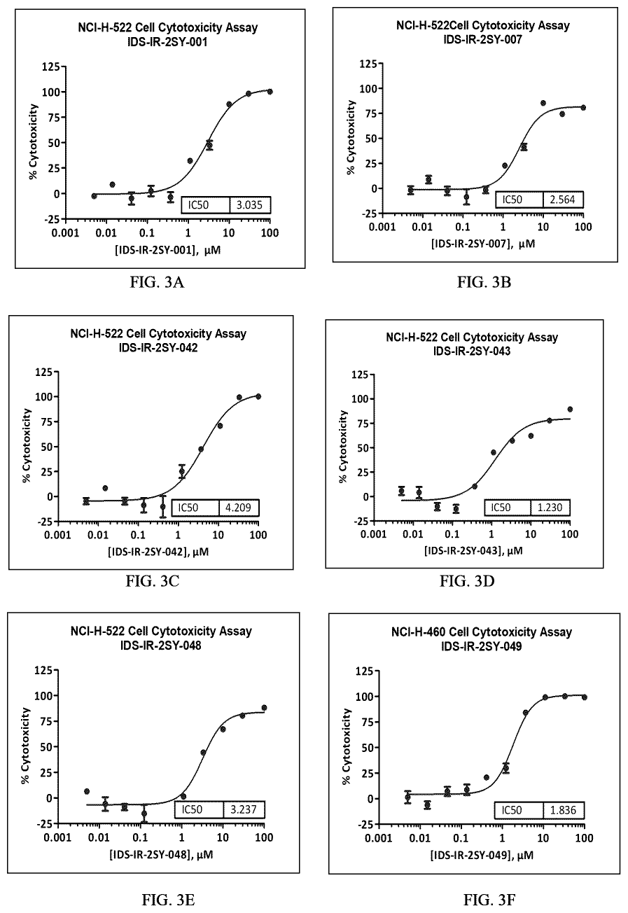

FIG. 1-3 provide the cell toxicity curve against the concentrations of Compound-A1 in various cell lines.

### REFERENCES

1. Kamb, A., et. al., Why is cancer drug discovery so difficult?Nat. Rev. Drug Discov. 6, 115-20 (2007)
2. Chin, L. & Gray, J. W. Translating insights from the cancer genome into clinical practice. Nature 452, 553-563 (2008)
3. Moffat, J. G., et. al., Opportunities and challenges in phenotypic drug discovery: an industry perspective. Nature Reviews Drug Discovery 16, 531-543 (2017)
4. Moffat, J. G., et. al., Phenotypic screening in cancer drug discovery—past, present and future. Nature Reviews Drug Discovery 13, 588-602 (2014)

1. A compound of Formula A, 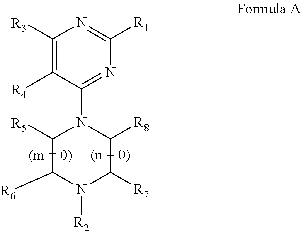
or stereoisomer, tautomers, solvates, hydrates, pharmaceutically acceptable salts thereof
Wherein:
R1 is selected from the group consisting of: 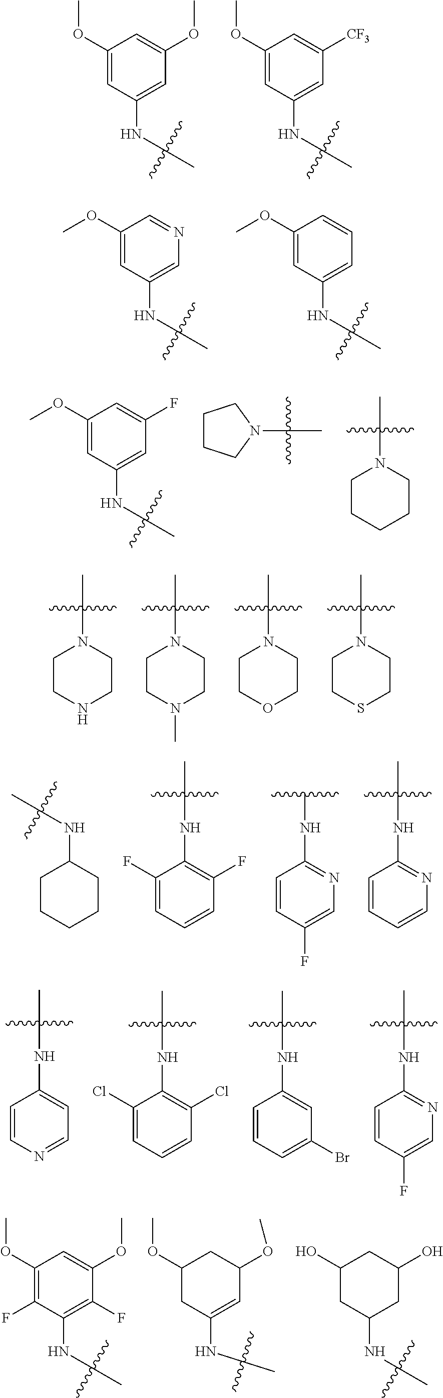 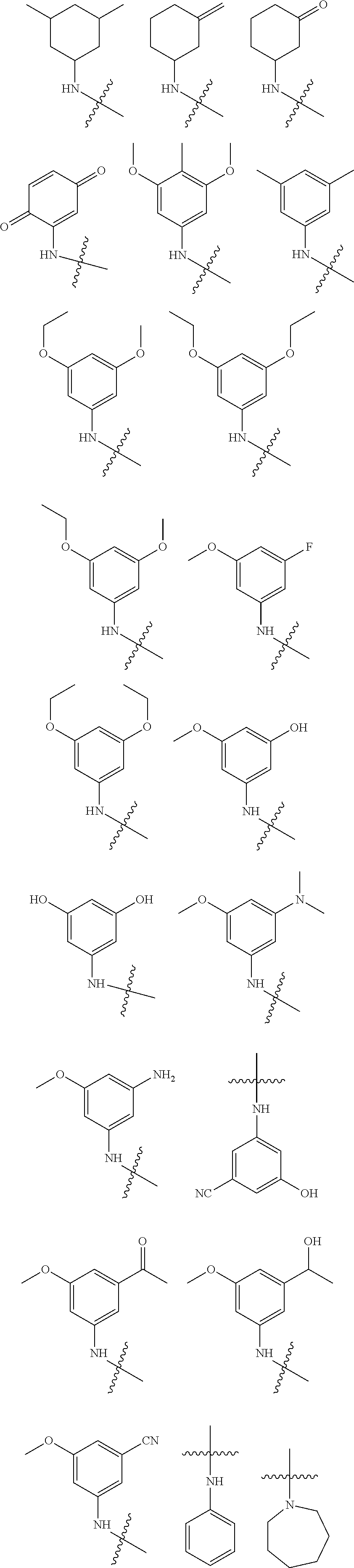
R2 is selected from the group consisting of: 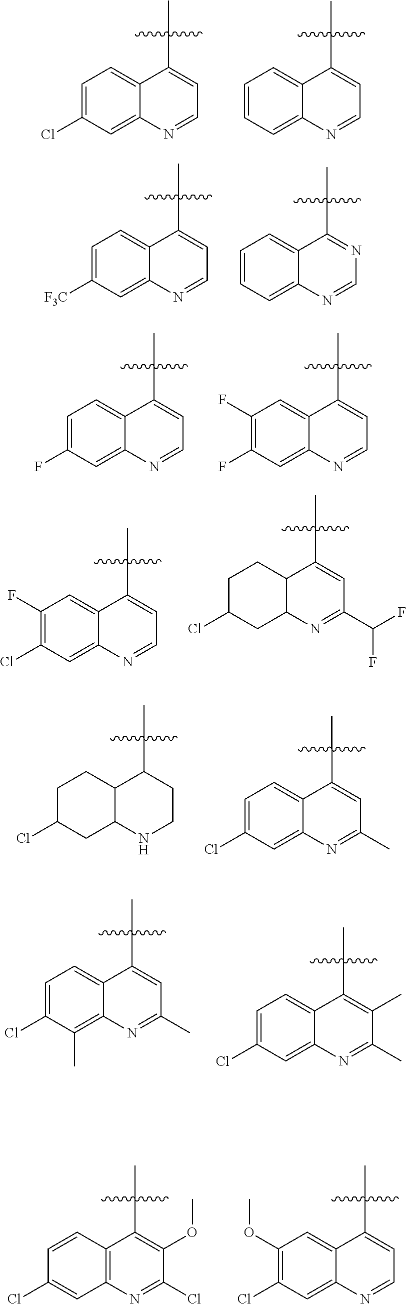 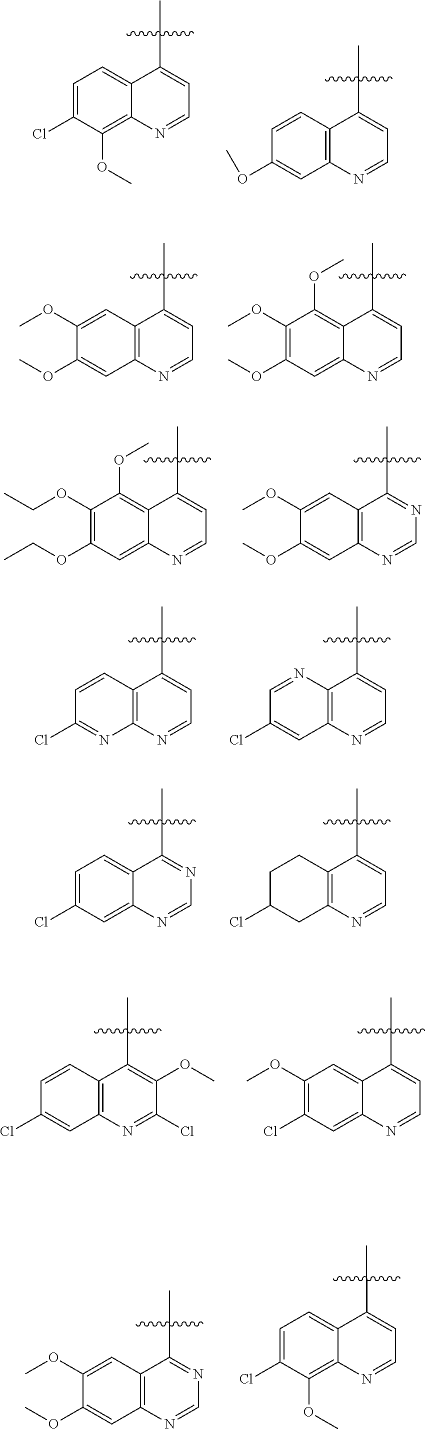 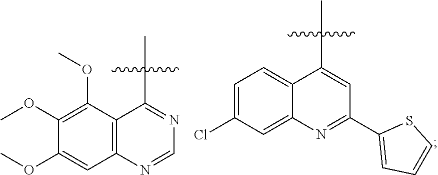
R3 is selected from CF₃, CH₃, H and F;
R4 is selected from H, F and CH₃;
R5 is selected from H and CH₃;
R6 is H;
R7 is selected from H and CH₃;
R8 is selected from H and CH₃; and
m=0, and n=0, wherein m and n indicate the number of carbons between the indicated bonds.

2. The compound as claimed in claim 1, wherein, R1 is 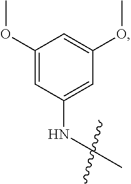 [SMILES: *Nc1cc(OC)cc(OC)c1]
R2 is 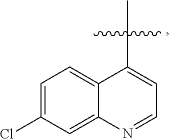 [SMILES: *c1ccnc2cc(Cl)ccc12]
R3 is CF₃, R4 is H, R5 is H, R6 is H, R7 is H, R8 is H, m=0 and n=0, having chemical structure as represented by Compound A1, 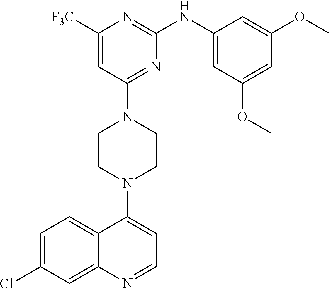 [SMILES: COc1cc(Nc2nc(N3CCN(c4ccnc5cc(Cl)ccc45)CC3)cc(C(F)(F)F)n2)cc(OC)c1]
Compound A1, 4-(4-(7-chloroquinolin-4-yl)piperazin-1-yl)-N-(3,5-dimethoxyphenyl)-6-(trifluoromethyl)pyridin-2-amine.

3. The compound as claimed in claim 1, wherein, R1 is 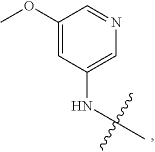 [SMILES: *Nc1cncc(OC)c1]
R2 is 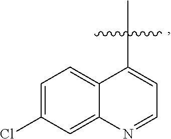 [SMILES: *c1ccnc2cc(Cl)ccc12]
R3 is CF₃, R4 is H, R5 is H, R6 is H, R7 is H, R8 is H, m=0 and n=0, having chemical structure as represented by Compound A7, 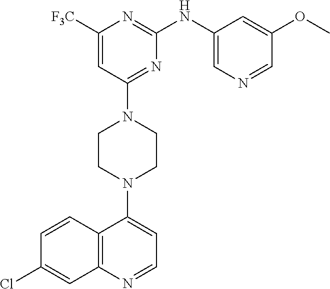 [SMILES: COc1cncc(Nc2nc(N3CCN(c4ccnc5cc(Cl)ccc45)CC3)cc(C(F)(F)F)n2)c1]
Compound A7, 4-(4-(7-chloroquinolin-4-yl)piperazin-1-yl)-N-(5-methoxypyridin-3-yl)-6-(trifluoromethyl)pyrimidin-2-amine.

4. The compound as claimed in claim 1, wherein, R1 is 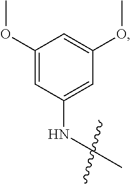 [SMILES: *Nc1cc(OC)cc(OC)c1]
R2 is 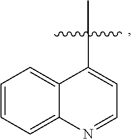 [SMILES: *c1ccnc2ccccc12]
R3 is CH₃, R4 is H, R5 is H, R6 is H, R7 is H, R8 is H, m=0 and n=0, having chemical structure as represented by Compound A42, 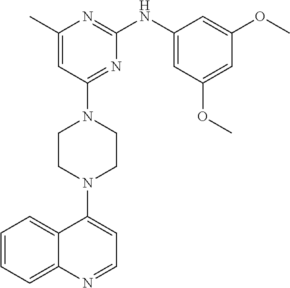 [SMILES: COc1cc(Nc2nc(C)cc(N3CCN(c4ccnc5ccccc45)CC3)n2)cc(OC)c1]
Compound A42, N-(3,5-dimethoxyphenyl)-4-methyl-6-(4-(quinolin-4-yl)piperazin-1-yl)pyrimidin-2-amine.

5. The compound as claimed in claim 1, wherein, R1 is 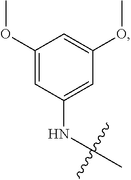 [SMILES: *Nc1cc(OC)cc(OC)c1]
R2 is 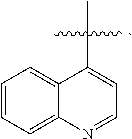 [SMILES: *c1ccnc2ccccc12]
R3 is CF₃, R4 is H, R5 is H, R6 is H, R7 is H, R8 is H, m=0 and n=0, having chemical structure as represented by Compound A43, 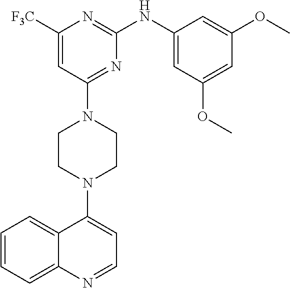 [SMILES: COc1cc(Nc2nc(N3CCN(c4ccnc5ccccc45)CC3)cc(C(F)(F)F)n2)cc(OC)c1]
Compound A43, N-(3,5-dimethoxyphenyl)-4-(4-(quinolin-4-yl)piperazin-1-yl)-6-(trifluoromethyl)pyrimidin-2-amine.

6. The compound as claimed in claim 1, wherein, R1 is 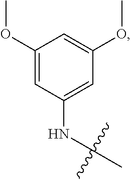 [SMILES: *Nc1cc(OC)cc(OC)c1]
R2 is 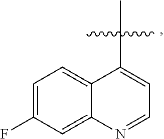 [SMILES: *c1ccnc2cc(F)ccc12]
R3 is CH₃, R4 is H, R5 is H, R6 is H, R7 is H, R8 is H, m=0 and n=0, having chemical structure as represented by Compound A48, 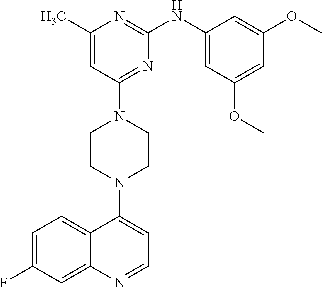 [SMILES: COc1cc(Nc2nc(C)cc(N3CCN(c4ccnc5cc(F)ccc45)CC3)n2)cc(OC)c1]
Compound A48, N-(3,5-dimethoxyphenyl)-4-(4-(7-fluoroquinolin-4-yl)piperazin-1-yl)-6-methylpyrimidin-2-amine.

7. The compound as claimed in claim 1, wherein, R1 is 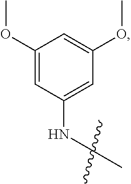 [SMILES: *Nc1cc(OC)cc(OC)c1]
R2 is 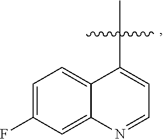 [SMILES: *c1ccnc2cc(F)ccc12]
R3 is CF₃, R4 is H, R5 is H, R6 is H, R7 is H, R8 is H, m=0 and n=0, having chemical structure as represented by Compound A49, 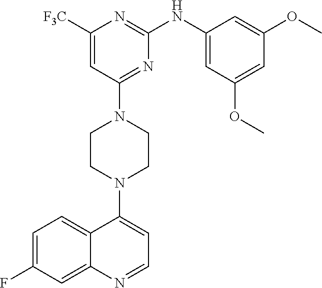 [SMILES: COc1cc(Nc2nc(N3CCN(c4ccnc5cc(F)ccc45)CC3)cc(C(F)(F)F)n2)cc(OC)c1]
Compound A49, N-(3,5-dimethoxyphenyl)-4-(4-(7-fluoroquinolin-4-yl)piperazin-1-yl)-6-(trifluoromethyl)pyrimidin-2-amine.

8. A pharmaceutical composition comprising a compound according to claim 1; and a pharmaceutically acceptable carrier, excipient, diluent, adjuvant, vehicle or a combination thereof.

9. A method of treating a disease, comprising administering an effective amount of a composition comprising a compound of Formula A, 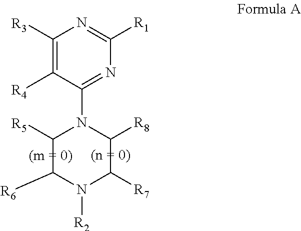
or stereoisomer, tautomers, solvates, hydrates, pharmaceutically acceptable salts thereof, wherein
R1 is selected from the group consisting of: 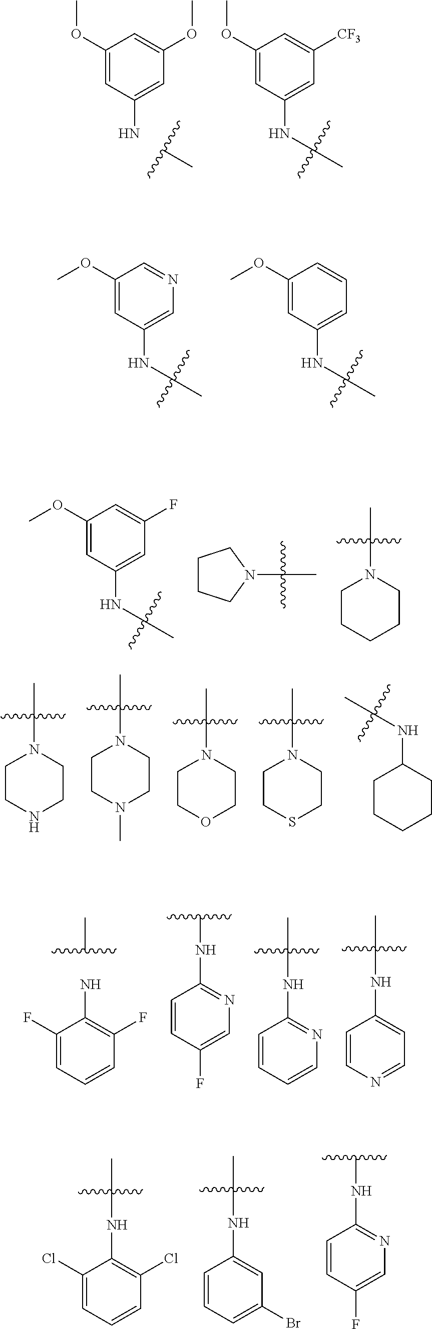 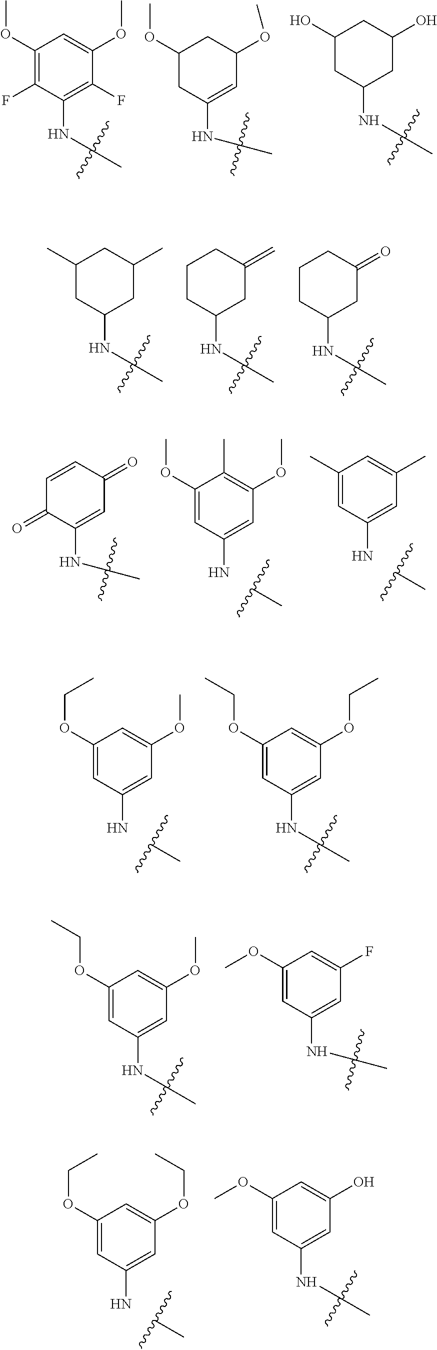 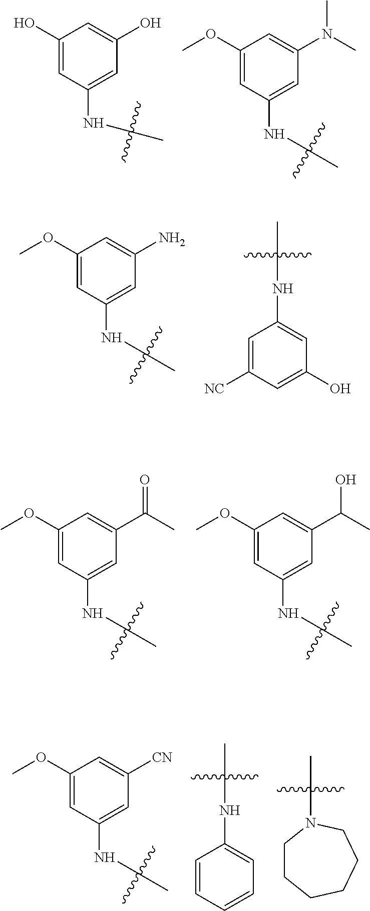
R2 is selected from the group consisting of: 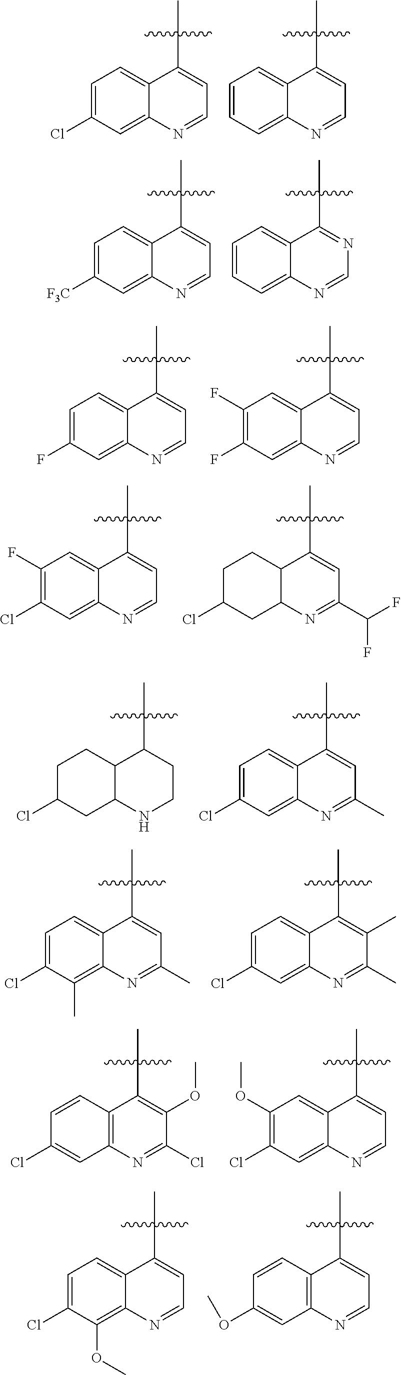 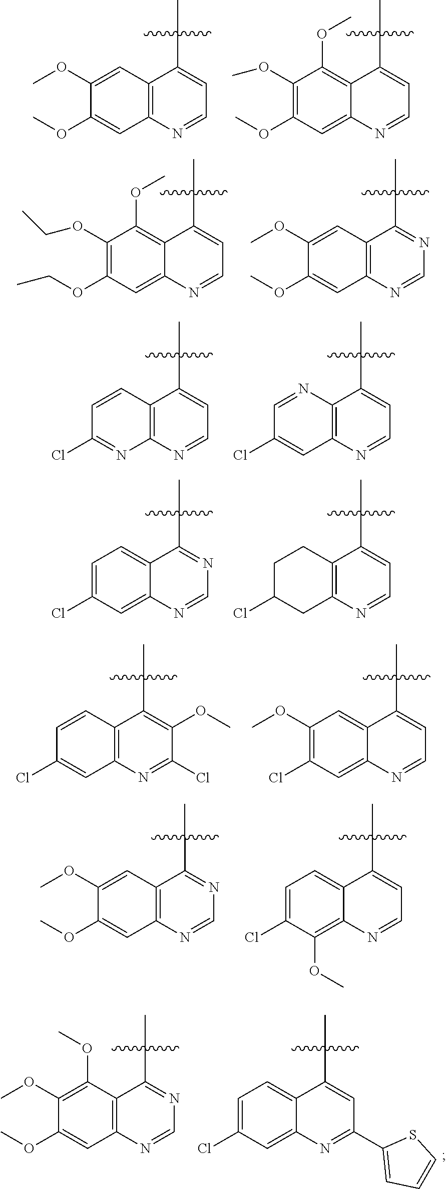
R3 is selected from CF₃, CH₃, H and F;
R4 is selected from H, F and CH₃;
R5 is selected from H and CH₃;
R6 is H;
R7 is selected from H and CH₃
R8 is selected from H and CH₃;
m=0, and n=0, wherein m and n indicate the number of carbons between the indicated bonds; and a pharmaceutically acceptable carrier, excipient, diluent, adjuvant, vehicle or a combination thereof to a person in need thereof, wherein, the disease is cancer including lung cancer.

10. A pharmaceutical composition comprising a compound according to claim 2 and a pharmaceutically acceptable carrier, excipient, diluent, adjuvant, vehicle or a combination thereof.

11. A pharmaceutical composition comprising a compound according to claim 3 and a pharmaceutically acceptable carrier, excipient, diluent, adjuvant, vehicle or a combination thereof.

12. A pharmaceutical composition comprising a compound according to claim 4 and a pharmaceutically acceptable carrier, excipient, diluent, adjuvant, vehicle or a combination thereof.

13. A pharmaceutical composition comprising a compound according to claim 5 and a pharmaceutically acceptable carrier, excipient, diluent, adjuvant, vehicle or a combination thereof.

14. A pharmaceutical composition comprising a compound according to claim 6 and a pharmaceutically acceptable carrier, excipient, diluent, adjuvant, vehicle or a combination thereof.

15. A pharmaceutical composition comprising a compound according to claim 7 and a pharmaceutically acceptable carrier, excipient, diluent, adjuvant, vehicle or a combination thereof.

16. A method of treating a disease, comprising administering an effective amount of the composition comprising a compound of Formula A, 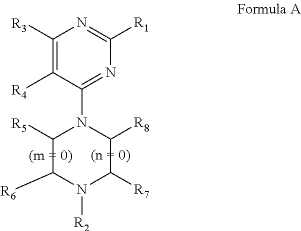
or stereoisomer, tautomers, solvates, hydrates, pharmaceutically acceptable salts thereof, wherein R1 is 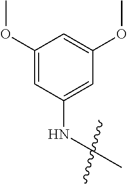 [SMILES: *Nc1cc(OC)cc(OC)c1]
R2 is 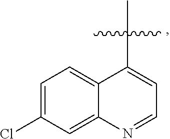 [SMILES: *c1ccnc2cc(Cl)ccc12]
R3 is CF₃, R4 is H, R5 is H, R6 is H, R7 is H, R8 is H, m=0 and n=0, having chemical structure as represented by Compound A1, 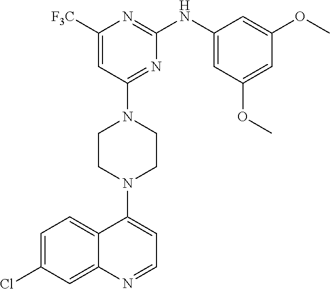 [SMILES: COc1cc(Nc2nc(N3CCN(c4ccnc5cc(Cl)ccc45)CC3)cc(C(F)(F)F)n2)cc(OC)c1]
4-(4-(7-chloroquinolin-4-yl)piperazin-1-yl)-N-(3,5-dimethoxyphenyl)-6-(trifluoromethyl)pyridin-2-amine; and a pharmaceutically acceptable carrier, excipient, diluent, adjuvant, vehicle or a combination thereof to a person in need thereof, wherein, the disease is cancer including lung cancer.
